(19) United States
(12) Patent Application Publication
(10) Pub. No.: US 2018/0085014 A1
(43) Pub. Date: Mar. 29, 2018

(54) PULSE WAVE MEASURING APPARATUS, METHOD FOR MEASURING PULSE WAVES, AND RECORDING MEDIUM

(71) Applicant: Panasonic Intellectual Property Management Co., Ltd., Osaka (JP)

(72) Inventors: KENTA MURAKAMI, Osaka (JP); MOTOTAKA YOSHIOKA, Osaka (JP); JUN OZAWA, Nara (JP)

(21) Appl. No.: 15/672,295

(22) Filed: Aug. 9, 2017

(30) Foreign Application Priority Data
Sep. 23, 2016 (JP) 2016-185686
May 24, 2017 (JP) 2017-102800

(51) Int. Cl. A61B 5/024; A61B 5/00

(52) U.S. Cl. CPC A61B 5/02433; A61B 5/0062; A61B 5/7275; A61B 5/6887; A61B 5/7282; A61B 5/7253

(57) ABSTRACT

A pulse wave measuring apparatus includes a processor and a memory. The processor instructs a lighting device outside thereof to cause the amplitude of a first hue waveform obtained from first visible light images to fall within a certain hue range, calculates a degree of correlation between a first visible light waveform obtained from first visible light images and a first infrared waveform obtained from first infrared images, outputs an infrared control signal and a visible light control signal for adjusting the amount of light of an infrared light source and the lighting device, respectively, in accordance with the degree of correlation, extracts a second visible light waveform and a second infrared waveform from second visible light images and second infrared images, respectively, calculates first biological information from feature values of at least either the second visible light waveform or the second infrared waveform, and outputs the first biological information.

### BACKGROUND

### 1. Technical Field

The present disclosure relates to a pulse wave measuring apparatus, a method for measuring pulse waves, and a recording medium that measure a person's pulse waves in a noncontact manner.

### 2. Description of the Related Art

Japanese Unexamined Patent Application Publication No. 2013-192620 discloses a technique for measuring a heart rate and the depth of sleep in a noncontact manner using millimeter waves, visible light, infrared light, or the like.

Japanese Unexamined Patent Application Publication No. 2004-146873 discloses a technique for appropriately switching an imaging apparatus from an infrared imaging mode, in which infrared light is radiated onto a subject, to a normal imaging mode.

### SUMMARY

The techniques disclosed in Japanese Unexamined Patent Application Publication No. 2013-192620 and Japanese Unexamined Patent Application Publication No. 2004-146873, however, require further improvements.

In one general aspect, the techniques disclosed here feature a pulse wave measuring apparatus including a processor. The processor obtains, from a lighting device provided outside the pulse wave measuring apparatus, a first control pattern specifying first correspondences, which indicate color temperatures of visible light output from the lighting device corresponding to a plurality of instructions, determines a first instruction corresponding to information indicating a first color temperature held by the pulse wave measuring apparatus while referring to the first control pattern, outputs the first instruction to the lighting device, obtains a plurality of first visible light images by capturing, in a visible light range, images of a user onto whom the lighting device is radiating visible light having the first color temperature corresponding to the first instruction, calculates a plurality of first hues from the plurality of first visible light images, extracts a first hue waveform from the plurality of first hues, determines, if amplitude of the first hue waveform does not fall within a certain hue range, a second instruction corresponding to a second color temperature, which is different from the first color temperature, while referring to the first control pattern, outputs the second instruction to the lighting device, obtains a plurality of second visible light images, by capturing, in the visible light range, images of the user onto whom the lighting device is radiating visible light having the second color temperature corresponding to the second instruction, calculates a plurality of second hues from the plurality of second visible light images, extracts a second hue waveform from the plurality of second hues, and performs, if amplitude of the second hue waveform falls within the certain hue range, a first process, wherein the first process includes a plurality of first infrared images are obtained by capturing, in an infrared range, images of the user onto whom an infrared light source is radiating infrared light, a first visible light waveform is extracted from the plurality of second visible light images, a first infrared waveform is extracted from the plurality of first infrared images, a degree of correlation between the extracted first visible light waveform and the extracted first infrared waveform is calculated, an infrared control signal for adjusting an amount of infrared light of the infrared light source is output to the infrared light source in accordance with the degree of correlation, a visible light control signal for adjusting an amount of visible light of the lighting device is output to the lighting device in accordance with the degree of correlation, a plurality of third visible light images are obtained by capturing, in the visible light range, images of the user onto whom the lighting device is radiating visible light based on the visible light control signal, a plurality of second infrared images are obtained by capturing, in the infrared range, images of the user onto whom the infrared light source is radiating infrared light based on the infrared control signal, a second visible light waveform is extracted from the plurality of third visible light images, a second infrared waveform is extracted from the plurality of second infrared images, first biological information is calculated from at least either a feature value of the second visible light waveform or a feature value of the second infrared waveform, and the calculated first biological information is output.

According to the present disclosure, further improvements can be achieved.

It should be noted this general or specific aspect may be implemented as a system, a method, an integrated circuit, a computer program, a computer-readable recording medium, or any selective combination thereof. The computer-readable recording medium may be, for example, a nonvolatile recording medium such as a compact disc read-only memory (CD-ROM).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a schematic diagram illustrating a situation in which a user uses a pulse wave measuring system according to an embodiment;

FIG. 2 is a block diagram illustrating an example of the hardware configuration of a pulse wave measuring apparatus;

FIG. 3 is a block diagram illustrating an example of the hardware configuration of a lighting device according to the embodiment;

FIG. 4 is a block diagram illustrating an example of the hardware configuration of a mobile terminal according to the embodiment;

FIG. 5A is a diagram illustrating an example of usage of the pulse wave measuring apparatus;

FIG. 5B is a diagram illustrating an example of usage of the pulse wave measuring apparatus;

FIG. 6 is a diagram illustrating an example of the usage of the pulse wave measuring apparatus;

FIG. 7 is a block diagram illustrating an example of the functional configuration of the pulse wave measuring apparatus according to the embodiment;

FIG. 8A is a graph illustrating an example of changes in luminance in visible light images according to the embodiment;

FIG. 8B a graph illustrating an example of changes in luminance in infrared images according to the embodiment;

FIG. 9A is a graph illustrating an example of calculation of pulse wave timings according to the embodiment;

FIG. 9B is a graph illustrating an example of pulse wave timings;

FIG. 10 is a graph illustrating an example of heartbeat intervals obtained over time;

FIG. 11A is a graph illustrating a visible light waveform obtained from visible light images;

FIG. 11B is a graph in which first derivatives of the visible light waveform;

FIG. 12 is a graph illustrating a visible light waveform whose gradients from top points to bottom points are calculated;

FIG. 13A is a graph an illustrating infrared waveform when an infrared camera has captured images of a person's skin;

FIG. 13B is a graph illustrating an infrared waveform when the infrared camera has captured images of the person's skin;

FIG. 13C is a graph illustrating an infrared waveform when the infrared camera has captured images of the person's skin;

FIG. 13D is a graph illustrating the infrared waveform when the infrared camera has captured images of a person's skin;

FIG. 14 is a graph in which first heartbeat intervals and second heartbeat intervals are plotted in chronological order;

FIG. 15A is a diagram illustrating a specific example of a determination whether heartbeat intervals are appropriate;

FIG. 15B is a graph illustrating an example of a visible light waveform or an infrared waveform;

FIG. 16 is a diagram illustrating an example of a case in which too many peaks have been obtained in a visible light waveform and too many peaks have not been obtained in a corresponding infrared waveform;

FIG. 17A is a graph illustrating peaks (top points) obtained from a visible light waveform;

FIG. 17B is a graph illustrating peaks (top points) obtained from an infrared waveform;

FIG. 18A is a diagram illustrating an example of a visible light waveform;

FIG. 18B is a diagram illustrating an example of a an infrared waveform;

FIG. 19 is a graph illustrating an example in which peaks obtained while the amount of light of a light source is being adjusted are not used for the calculation of a degree of correlation between a visible light waveform and an infrared waveform;

FIG. 20 is a diagram illustrating an example of simplest steps in which the pulse wave measuring apparatus decreases the amount of light of a visible light source to zero and increases the amount of light of the infrared light source to an appropriate value;

FIG. 21 is a graph illustrating the adjustment of the amount of light that is not performed until two or more successive certain feature points are extracted from a visible light waveform or an infrared waveform in a second certain time period;

FIG. 22 is a diagram illustrating a difference in how a visible light imaging unit captures an image of the user's face depending on color temperature;

FIG. 23 is a diagram illustrating a process for calculating a hue signal of a hue from RGB luminance signals;

FIG. 24 is a diagram illustrating a color wheel;

FIG. 25 is a diagram illustrating hue waveforms obtained after RGB (red, green, and blue) luminance signals are converted using different hue ranges;

FIG. 26A is a graph illustrating changes in voltage according to an amount of light of a visible light source and an amount of light of an infrared light source;

FIG. 26B is a graph illustrating a visible light waveform and an infrared waveform when voltages applied to the light sources are changed;

FIG. 26C is a graph illustrating a visible light waveform and an infrared waveform when voltages applied to the light sources are changed;

FIG. 27A is a graph illustrating changes in voltage according to an amount of light of a visible light source and an amount of light of an infrared light source;

FIG. 27B is a graph illustrating a visible light waveform and an infrared waveform when voltages applied to light sources are changed;

FIG. 28A is a graph illustrating changes in voltage according to an amount of light of a lighting device and an amount of light of an infrared light source;

FIG. 28B is a graph illustrating a visible light waveform and an infrared waveform when voltages applied to light sources are changed;

FIG. 29A is a graph illustrating changes in voltage according to an amount of light of a lighting device and an amount of light of an infrared light source;

FIG. 29B is a graph illustrating a visible light waveform and an infrared waveform when voltages applied to light sources are changed;

FIG. 30A is a graph illustrating changes in voltage according to an amount of light of the lighting device and an amount of light of the infrared light source;

FIG. 30B is a graph illustrating a visible light waveform and an infrared waveform when voltages applied to light sources are changed;

FIG. 31 is a diagram illustrating an example of a screen of a display device;

FIG. 32 is a flowchart illustrating a process performed by the pulse wave measuring apparatus according to the embodiment;

FIG. 33 is a flowchart illustrating details of a process for determining whether too many peaks have been obtained according to the embodiment;

FIG. 34 is a flowchart illustrating details of a process for calculating a degree of correlation according to the embodiment;

FIG. 35 is a flowchart illustrating details of a process for adjusting the amount of light according to the embodiment; and

FIG. 36 is a flowchart illustrating a process for identifying a control pattern according to a modification.

### DETAILED DESCRIPTION

### Underlying Knowledge Forming Basis of Present Disclosure

The present inventor has identified the following problems in the techniques disclosed in the examples of the related art.

Japanese Unexamined Patent Application Publication No. 2013-192620 does not explain about adjustment of the amount of light of an infrared light source at a time when pulse waves are obtained in a darkroom, and it is difficult to measure a heart rate or pulse waves in a noncontact manner in a darkroom.

In Japanese Unexamined Patent Application Publication No. 2004-146873, a mode is switched using a ratio of the luminance of visible light to the luminance of infrared light, but in a darkroom, it is not easy to measure pulse waves if the mode is switched using the ratio of luminance.

The present disclosure provides a pulse wave measuring apparatus and the like capable of accurately measuring pulse waves in a darkroom.

A pulse wave measuring apparatus according to an aspect of the present disclosure is a pulse wave measuring apparatus including a processor. The processor obtains, from a lighting device provided outside the pulse wave measuring apparatus, a first control pattern specifying first correspondences, which indicate color temperatures of visible light output from the lighting device corresponding to a plurality of instructions, determines a first instruction corresponding to information indicating a first color temperature held by the pulse wave measuring apparatus while referring to the first control pattern, outputs the first instruction to the lighting device, obtains a plurality of first visible light images by capturing, in a visible light range, images of a user onto whom the lighting device is radiating visible light having the first color temperature corresponding to the first instruction, calculates a plurality of first hues from the plurality of first visible light images, extracts a first hue waveform from the plurality of first hues, determines, if amplitude of the first hue waveform does not fall within a certain hue range, a second instruction corresponding to a second color temperature, which is different from the first color temperature, while referring to the first control pattern, outputs the second instruction to the lighting device, obtains a plurality of second visible light images, by capturing, in the visible light range, images of the user onto whom the lighting device is radiating visible light having the second color temperature corresponding to the second instruction, calculates a plurality of second hues from the plurality of second visible light images, extracts a second hue waveform from the plurality of second hues, and performs, if amplitude of the second hue waveform falls within the certain hue range, a first process, wherein the first process includes a plurality of first infrared images are obtained by capturing, in an infrared range, images of the user onto whom an infrared light source is radiating infrared light, a first visible light waveform is extracted from the plurality of second visible light images, a first infrared waveform is extracted from the plurality of first infrared images, a degree of correlation between the extracted first visible light waveform and the extracted first infrared waveform is calculated, an infrared control signal for adjusting an amount of infrared light of the infrared light source is output to the infrared light source in accordance with the degree of correlation, a visible light control signal for adjusting an amount of visible light of the lighting device is output to the lighting device in accordance with the degree of correlation, a plurality of third visible light images are obtained by capturing, in the visible light range, images of the user onto whom the lighting device is radiating visible light based on the visible light control signal, a plurality of second infrared images are obtained by capturing, in the infrared range, images of the user onto whom the infrared light source is radiating infrared light based on the infrared control signal, a second visible light waveform is extracted from the plurality of third visible light images, a second infrared waveform is extracted from the plurality of second infrared images, first biological information is calculated from at least either a feature value of the second visible light waveform or a feature value of the second infrared waveform, and the calculated first biological information is output.

In this aspect, the color temperature of the lighting device provided outside is adjusted such that the amplitude of the hue waveform obtained from the plurality of first visible light images falls within the certain hue range, and the user's pulse waves are extracted from the plurality of second visible light images and the plurality of first infrared images obtained after the color temperature of the lighting device is adjusted. As a result, clear first and second hue waveforms that are hardly affected by noise caused by changes in luminance can be obtained.

Furthermore, in this aspect, the degree of correlation between the first visible light waveform obtained from the plurality of second visible light images and the first infrared waveform obtained from the plurality of first infrared images is calculated, and the amount of visible light of the lighting device and the amount of infrared light of the infrared light source are adjusted in accordance with the degree of correlation. As a result, even if a commercial lighting device is used, for example, the amount of visible light and the amount of infrared light can be appropriately adjusted, and the biological information can be accurately calculated.

In addition, the certain hue range may be a range of hues of 0 to 60 degrees. In addition, a hue of 30 degrees may serve as a reference for the certain hue range.

By adjusting the color temperature of the lighting device such that the color of a surface of the user's skin changes from white to a reddish color, especially such that a hue H becomes close to 30 degrees, for example, the first and second hue waveforms can be obtained more robustly against body movement and environmental noise. As a result, clear first and second hue waveforms that are hardly affected by noise caused by changes in luminance can be obtained.

In addition, in the calculation of the degree of correlation, the processor may (1) extract a plurality of first peaks in a plurality of first unit periods included in a plurality of first unit waveforms, the plurality of first peaks being a plurality of first maximum points included in the plurality of first unit waveforms or a plurality of first minimum points included in the plurality of first unit waveforms, the first visible light waveform including the plurality of first unit waveforms, the plurality of first maximum points and the plurality of first unit waveforms corresponding to each other, the plurality of first minimum points and the plurality of first unit waveforms corresponding to each other, and the plurality of first unit waveforms and the plurality of first unit periods corresponding to each other, (2) extract a plurality of second peaks in a plurality of second unit periods included in a plurality of second unit waveforms, the plurality of second peaks being a plurality of second maximum points included in the plurality of second unit waveforms or a plurality of second minimum points included in the plurality of second unit waveforms, the first infrared waveform including the plurality of second unit waveforms, the plurality of second maximum points and the plurality of second unit waveforms corresponding to each other, the plurality of second minimum points and the plurality of second unit waveforms corresponding to each other, and the plurality of second unit waveforms and the plurality of second unit periods corresponding to each other, (3) calculate a plurality of first heartbeat intervals on the basis of the plurality of first unit periods, the plurality of first heartbeat intervals being intervals between first time points and second time points, the plurality of first unit periods including the first time points and the second time points, and time included in the plurality of first unit periods not existing between the first time points and the second time points, (4) calculate a plurality of second heartbeat intervals on the basis of the plurality of second unit periods, the plurality of second heartbeat intervals being intervals between third time points and fourth time points, the plurality of second unit periods including the third time points and the fourth time points, and time included in the plurality of second unit periods not existing between the third time points and the fourth time points, and calculate the degree of correlation using a following expression (1):

$$ρ1 = \frac{{σ}_{12}}{{σ}_{1} {σ}_{2}} \qquad ( 1 )$$

ρ1: First correlation coefficient
σ₁₂: Covariance between plurality of first heartbeat intervals and plurality of second heartbeat intervals
σ₁: First standard deviation, standard deviation of plurality of first heartbeat intervals
σ₂: Second standard deviation, standard deviation of plurality of second heartbeat intervals

In addition, the processor may calculate second biological information from at least either a feature value of the first visible light waveform and a feature value of the first infrared waveform and outputs the calculated second biological information.

In this case, the second biological information can be calculated from at least either the feature value of the first visible light waveform or the feature value of the first infrared waveform obtained before the amount of visible light or the amount of infrared light is adjusted, and the calculated second biological information can be output.

In addition, if the lighting device is a device whose amount of light is adjusted using a second control pattern, in which the amount of light is adjusted in one stage, namely on and off, the processor may output, to the infrared light source as the infrared control signal, a control signal for increasing the amount of infrared light of the infrared light source by a predetermined first value and, to the lighting device as the visible light control signal, a control signal for turning off the lighting device.

As a result, even if the lighting device is a device whose amount of light is adjusted in one stage, the amount of visible light and the amount of infrared light can be appropriately adjusted.

In addition, if the lighting device is a device whose amount of light is adjusted using a third control pattern, in which the amount of light is adjusted in two stages, namely using a first amount of visible light and a second amount of visible light, which is smaller than the first amount of visible light, the processor may output, to the infrared light source as the infrared control signal, a control signal for adjusting the amount of infrared light of the infrared light source from a first amount of infrared light to a second amount of infrared light, which is larger than the first amount of infrared light by a predetermined second value, and, to the lighting device as the visible light control signal, a control signal for adjusting the amount of visible light of the lighting device from the first amount of visible light to the second amount of visible light, determine a third value for the amount of infrared light in accordance with a change in luminance of infrared light obtained from the first and second infrared images and a change in luminance of visible light obtained from the first and third visible light images, and output, to the infrared light source as the infrared control signal, a control signal for adjusting the amount of infrared light of the infrared light source from the second amount of infrared light to a third amount of infrared light, which is larger than the second amount of infrared light by the determined third value, and, to the lighting device as the visible light control signal, a second-stage control signal for turning off the lighting device.

In this case, if the lighting device is a device whose amount of light is adjusted in two stages, the pulse wave measuring apparatus can obtain the infrared light waveform more effectively by obtaining, in the adjustment of the amount of light in a first stage, the amount of decrease in the luminance of visible light and increasing the amount of infrared light of the infrared light source in accordance with the obtained amount of decrease.

In addition, if the lighting device is a device whose amount of light is adjusted using a fourth control pattern, in which the amount of light is adjusted without stages, and if the calculated degree of correlation is equal to or higher than a certain threshold, the processor may output, to the infrared light source as the infrared control signal, a control signal for increasing the amount of infrared light of the infrared light source and, to the lighting device as the visible light control signal, a control signal for decreasing the amount of visible light of the lighting device, repeatedly perform the obtaining of the third visible light images, the extraction of the second visible light waveform, the obtaining of the second infrared images, the extraction of the second infrared light waveform, and the calculation of a degree of correlation, and, if the amount of visible light of the lighting device becomes equal to or smaller than a second threshold, and if the degree of correlation obtained as a result of the repeatedly performed calculation of a degree of correlation becomes equal to or higher than the certain threshold, output, to the lighting device as the visible light control signal, a control signal for turning off the lighting device.

In this case, the lighting device can be turned off more promptly compared to when the amount of visible light is linearly reduced to zero, thereby allowing the user to fall asleep more comfortably.

In addition, if the lighting device is a device whose amount of light is adjusted using a fourth control pattern, in which the amount of light is adjusted without stages and if the calculated degree of correlation is equal to or higher than a certain threshold, the processor may perform (i) a normal process, in which a control signal for increasing the amount of infrared light of the infrared light source at a first speed is output to the infrared light source as the infrared control signal, a control signal for decreasing the amount of visible light of the lighting device by a second speed is output to the lighting device as the visible light control signal, and the obtaining of the third visible light images, the extraction of the second visible light waveform, the obtaining of the second infrared images, the extraction of the second infrared light waveform, and the calculation of a degree of correlation are repeatedly performed, or (ii) a time-saving process, in which a control signal for increasing the amount of infrared light of the infrared light source at a third speed, which is twice or more higher than the first speed, is output to the infrared light source as the infrared control signal, a control signal for decreasing the amount of visible light of the lighting device at a fourth speed, which is twice or more higher than the second speed, is output to the lighting device as the visible light control signal, and the obtaining of the third visible light images, the extraction of the second visible light waveform, the obtaining of the second infrared images, the extraction of the second infrared light waveform, and the calculation of a degree of correlation are repeatedly performed.

As a result, the time taken to complete the switching operation can be reduced.

It should be noted that these general or specific aspects may be implemented as a system, a method, an integrated circuit, a computer program, a computer-readable storage medium such as a CD-ROM, or any selective combination thereof.

### Embodiment

In an embodiment, a pulse wave measuring apparatus will be described that obtains a user's pulse waves from visible light images and infrared images of the user and that controls light sources on the basis of a degree of correlation between feature values of the obtained pulse waves.

### 1-1. Configuration

### 1-1-1. Pulse Wave Measuring System

The configuration of a pulse wave measuring system according to the present embodiment will be described.

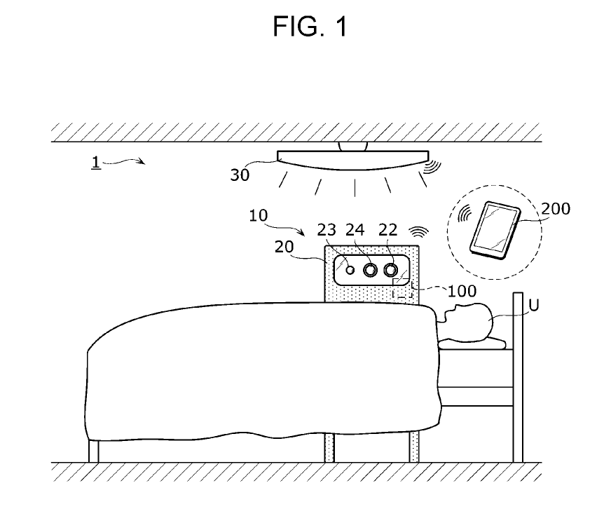

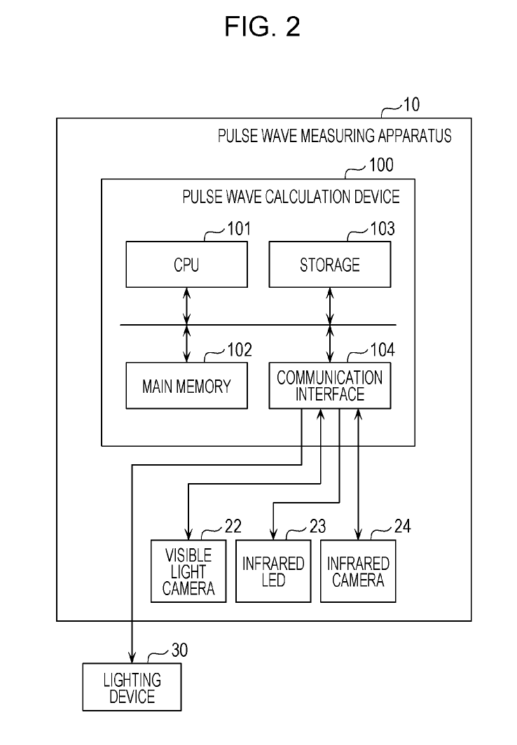

FIG. 1 is a schematic diagram illustrating a situation in which a user U uses a pulse wave measuring system 1 according to the present embodiment. FIG. 2 is a block diagram illustrating an example of the hardware configuration of a pulse wave measuring apparatus 10.

The pulse wave measuring system 1 includes the pulse wave measuring apparatus 10 and a lighting device 30. The pulse wave measuring system 1 may further include a mobile terminal 200. The pulse wave measuring apparatus 10, the lighting device 30, and the mobile terminal 200 are communicably connected to one another.

The pulse wave measuring apparatus 10 includes a visible light camera 22, an infrared light-emitting diode (LED) 23, an infrared camera 24, and a pulse wave calculation device 100.

As illustrated in FIG. 1, the pulse wave measuring apparatus 10 includes a case 20, and components illustrated in FIG. 2 are provided on a surface (e.g., a bottom surface) of the case 20 from which light is radiated. More specifically, in the pulse wave measuring apparatus 10, for example, the visible light camera 22, the infrared LED 23, and the infrared camera 24 are arranged next to one another in an upper part of a side surface of the case 20. In the pulse wave measuring apparatus 10, the pulse wave calculation device 100 obtains the user's pulse waves using images captured by the visible light camera 22 and the infrared camera 24 and controls the amount of light of the lighting device 30 and the amount of light of the infrared LED 23 on the basis of a degree of correlation between the obtained pulse waves.

The visible light camera 22 senses visible light. The visible light camera 22 is, for example, includes an image sensor such as a charge-coupled device (CCD) or a complementary metal-oxide-semiconductor (CMOS) image sensor. The visible light camera 22 uses an RGB color filter for the image sensor to cause the image sensor to obtain visible light, that is, light in a wavelength range of 400 to 800 nm, as RGB signals.

The infrared LED 23 is a light source that radiates infrared light. Infrared light is light having wavelengths in an infrared range (e.g., 800 to 2,500 nm). The infrared LED 23 may include bullet-shaped LEDs, surface-mount device (SMD) LEDs, or chip-on-board (COB) LEDs, that is, the infrared LED 23 may include a plurality of LEDs.

The infrared camera 24 senses infrared light. The infrared camera 24 may sense electromagnetic waves in a wavelength range (e.g., 700 to 900 nm) including a part of a visible light range. The infrared camera 24 is arranged next to the infrared LEDs 23. The infrared camera 24 includes a filter different from that used in the visible light camera 22 to cause an image sensor included therein to obtain infrared light, that is, light in a wavelength range of 800 nm and higher, as a monochromatic signal.

The pulse wave calculation device 100 is arranged inside the case 20. The pulse wave calculation device 100 includes a central processing unit (CPU) 101, a main memory 102, a storage 103, and a communication interface 104.

The CPU 101 is a processor that executes control programs stored in the storage 103 and the like.

The main memory 102 is a volatile storage area (main storage device) used as a working area when the CPU 101 executes the control programs.

The storage 103 is a nonvolatile storage device (auxiliary storage device) storing control programs and various pieces of data.

The communication interface 104 communicates data with other devices through a network. More specifically, the communication interface 104 outputs control signals to the lighting device 30, the visible light camera 22, the infrared LED 23, and the infrared camera 24 to control these devices. The communication interface 104 obtains image data obtained by the visible light camera 22 and the infrared camera 24.

The communication interface 104 may transmit a control signal to the lighting device 30. More specifically, the communication interface 104 may transmit a control signal to the lighting device 30 through infrared radiation.

The communication interface 104 may be communicably connected to the mobile terminal 200. More specifically, the communication interface 104 may be a wireless local area network (LAN) interface according to an Institute of Electrical and Electronics Engineers (IEEE) 802.11a, b, or n standard or a wireless communication interface according to a Bluetooth (registered trademark) standard.

### 1-1-2. Lighting Device

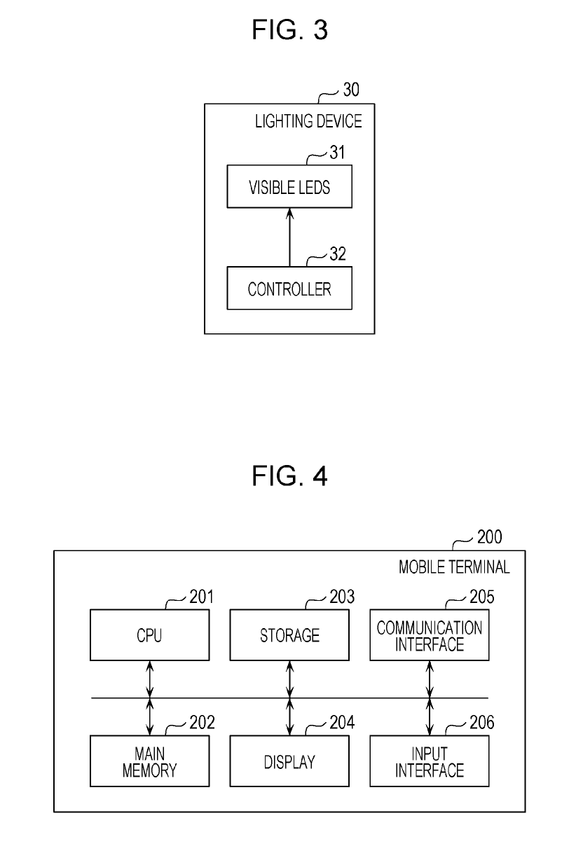

The hardware configuration of the lighting device 30 will be described with reference to FIG. 3.

FIG. 3 is a block diagram illustrating an example of the hardware configuration of the lighting device 30 according to the present embodiment.

The lighting device 30 is a light source that radiates visible light and includes visible LEDs 31 and a controller 32. The lighting device 30 receives a certain control signal transmitted from a remote control or the like and radiates varying amounts of light according to the certain control signal. The lighting device 30 may be, for example, a commercial lighting device such as a ceiling light, a pendant light, a bracket light, a stand light, a footlight, a spotlight, or a downlight or may be a device such as an LED light bulb, a linear tube LED lamp, or a circle (ring) LED lamp configured to be able to receive a control signal from the remote control.

The visible LEDs 31 are, for example, white LEDs. Visible light is light having wavelengths in a visible light range (e.g., 400 to 800 nm). The visible LEDs 31 are arranged, for example, on the bottom surface of a case in the shape of a ring. The visible LEDs 31 may be bullet-shaped LEDs, SMD LEDs, or COB LEDs. The visible LEDs 31 need not necessarily be arranged in the shape of a ring. The lighting device 30 may include a fluorescent light, a fluorescent light bulb, or a light bulb as the light source thereof instead of the visible LEDs 31.

The controller 32 receives a control signal transmitted from the certain remote control, the pulse wave measuring apparatus 10, or the mobile terminal 200 and adjusts the amount of light of the visible LEDs 31 in accordance with the received control signal. The controller 32 is achieved, for example, by a microcontroller and a communication module. The communication module may receive a control signal through infrared radiation, a wireless LAN, or Bluetooth (registered trademark).

### 1-1-3. Mobile Terminal

The hardware configuration of the mobile terminal 200 will be described with reference to FIG. 4.

FIG. 4 is a block diagram illustrating an example of the hardware configuration of the mobile terminal 200 according to the present embodiment.

As illustrated in FIG. 4, the mobile terminal 200 includes a CPU 201, a main memory 202, a storage 203, a display 204, a communication interface 205, and an input interface 206. The mobile terminal 200 is a communicable information terminal such as a smartphone or a tablet terminal.

The CPU 201 is a processor that executes control programs stored in the storage 203 and the like.

The main memory 202 is a volatile storage area (main storage device) used as a working area when the storage 203 executes the control programs.

The storage 203 is a nonvolatile storage area (auxiliary storage device) storing control programs and various pieces of data.

The display 204 is a display device that displays results of processing performed by the CPU 201. The display 204 is, for example, a liquid crystal display or an organic electroluminescent (EL) display.

The communication interface 205 is used to communicate with the pulse wave measuring apparatus 10. The communication interface 205 may be, for example, a wireless LAN interface according to an IEEE 802.11a, b, g, or n standard or may be a wireless communication interface according to a Bluetooth (registered trademark) standard. Alternatively, the communication interface 205 may be a wireless communication interface according to a communication standard used in a mobile communication system such as a third generation (3G) mobile communication system, a fourth generation (4G) mobile communication system, or long-term evolution (LTE; registered trademark).

The input interface 206 is, for example, a touch panel that is arranged on a front surface of the display 204 and that receives an input from the user who uses a user interface (UI) displayed on the display 204. The input interface 206 may be an input device such as a numeric keypad or a keyboard, instead.

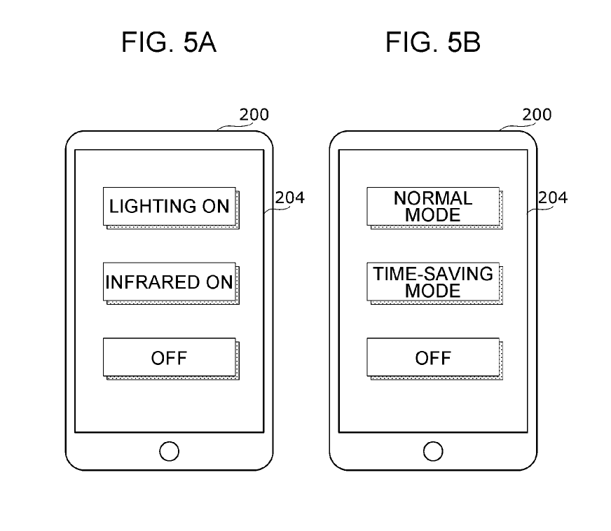

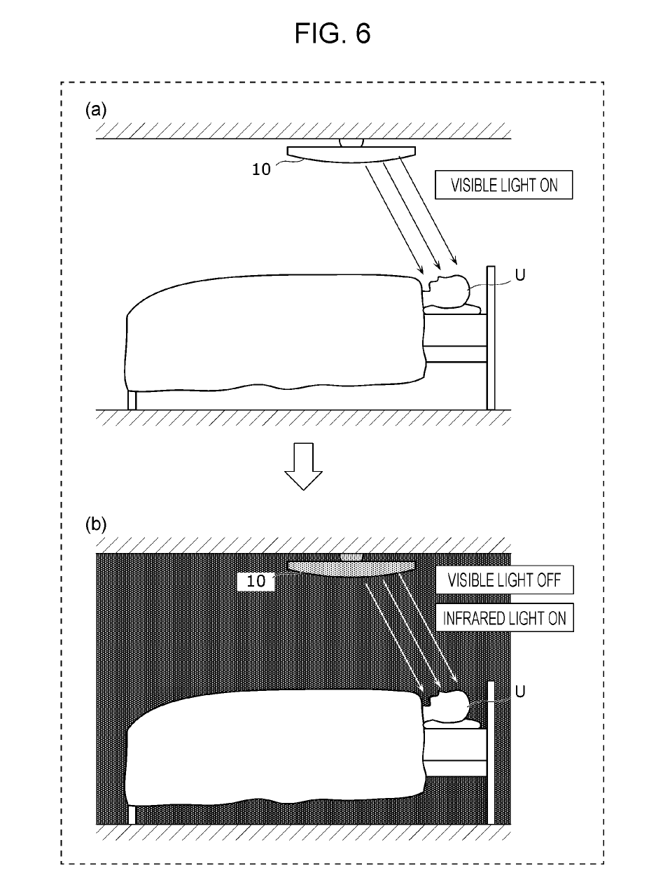

FIGS. 5A, 5B and 6 are diagrams illustrating examples of usage of the pulse wave measuring apparatus 10.

As illustrated in FIG. 5A and FIG. 5B, the mobile terminal 200 may display, on the display 204, UIs for operating the pulse wave measuring apparatus 10. The mobile terminal 200 may transmit a control signal to the pulse wave measuring apparatus 10 in accordance with an input based on one of the UIs.

In the pulse wave measuring system 1, the user can use the mobile terminal 200 as means for turning on and off the lighting device 30 and the infrared LED 23. If a remote control application for controlling the pulse wave measuring apparatus 10 is activated on the mobile terminal 200, for example, the mobile terminal 200 can be used as a remote control for the pulse wave measuring apparatus 10 and the lighting device 30. As illustrated in FIG. 5A, the user can turn on the lighting device 30 by selecting “lighting on”.

FIG. 6(a) illustrates an example of a situation in which the lighting device 30 is on. If the user selects “infrared on”, the infrared LED 23 is turned on regardless of whether the lighting device 30 is on or off. FIG. 6(b) illustrates a situation in which the lighting device 30 is off but the infrared LED 23 is on. Since the user does not sense infrared light radiated from the infrared LED 23, the user can fall asleep as usual. If the user selects “off”, both the lighting device 30 and the infrared LED 23 turn off, and any kind of light is not radiated onto the user.

If the user selects “normal mode” among the UIs illustrated in FIG. 5B, the amount of light of the lighting device 30 that has been on gradually decreases to zero, and the infrared LED 23 that has been off turns on and the amount of light of the infrared LED 23 gradually increases to an optimal value. As a result, the user's pulse waves can be obtained even during sleep.

If the user selects “time-saving mode”, the amount of light of the lighting device 30 decreases twice as fast as when the user has selected “normal mode”, and the amount of light of the infrared LED 23 increases twice as fast as when the user has selected “normal mode”. As a result, a period for which the lighting device 30 remains on becomes shorter than in the normal mode. Details of the time-saving mode will be described later.

### 1-2. Functional Configuration

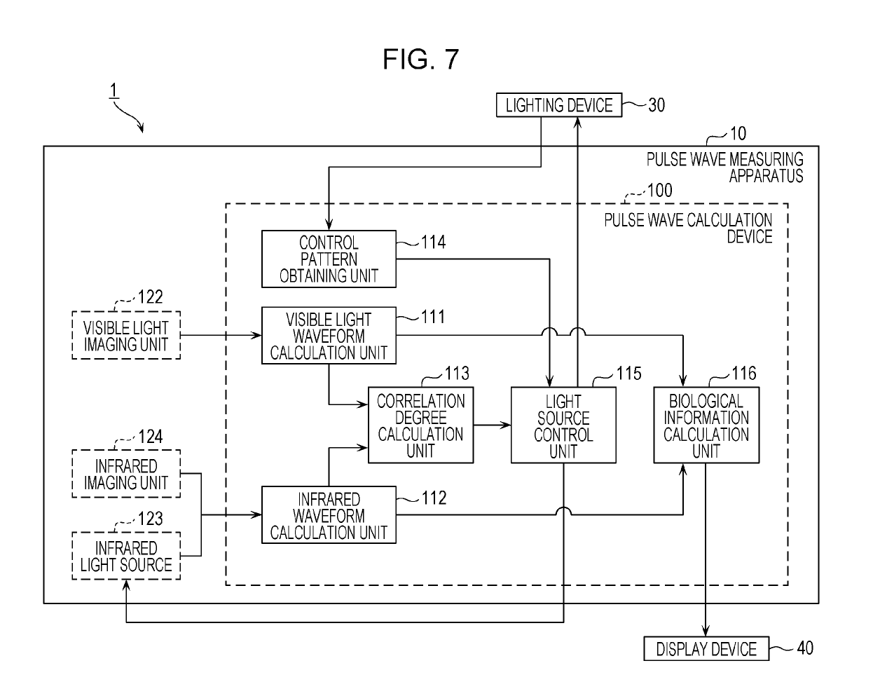

Next, the functional configuration of the pulse wave measuring apparatus 10 will be described with reference to FIG. 7.

FIG. 7 is a block diagram illustrating an example of the functional configuration of the pulse wave measuring apparatus 10 according to the present embodiment.

As illustrated in FIG. 7, the pulse wave measuring apparatus 10 includes a visible light imaging unit 122, an infrared light source 123, an infrared imaging unit 124, and the pulse wave calculation device 100.

The visible light imaging unit 122 captures an image of a target onto which the lighting device 30 is radiating visible light. More specifically, the visible light imaging unit 122 outputs, to a visible light waveform calculation unit 111 of the pulse wave calculation device 100, a visible light image obtained by capturing an image of the user's skin, which is the target, in the visible light range (e.g., in color). The visible light imaging unit 122 outputs a skin image obtained by capturing an image of a part of a person's skin including the person's face or hand, for example, as a visible light image. The visible light imaging unit 122 outputs a plurality of visible light images captured at a plurality of different timings, for example, to the visible light waveform calculation unit 111. Skin images are images of the same part of a person's skin including the person's face or hand captured at a plurality of temporally successive timings and are moving image or a plurality of still images. The visible light imaging unit 122 is achieved, for example, by the visible light camera 22.

The infrared light source 123 radiates infrared light onto the user. The amount of light radiated is adjusted by a light source control unit 115 of the pulse wave calculation device 100. The infrared light source 123 is achieved, for example, by the infrared LED 23.

The infrared imaging unit 124 captures, in the infrared range, an image of a target onto which the infrared light source 123 is radiating infrared light. More specifically, the infrared imaging unit 124 outputs, to an infrared waveform calculation unit 112 of the pulse wave calculation device 100, an infrared image obtained by capturing the user's skin, which is the target, in the infrared range (e.g., in monochrome). The infrared imaging unit 124 outputs, to the infrared waveform calculation unit 112, a plurality of infrared images captured at a plurality of different timings. The infrared imaging unit 124 captures an image of the same part as that whose image is captured by the visible light imaging unit 122. The infrared imaging unit 124 outputs a skin image obtained by capturing a part of a person's skin including the person's face or hand, for example, as an infrared image. This is because if the infrared imaging unit 124 captures an image of the same part as that whose image is captured by the visible light imaging unit 122, similar pulse waves can be obtained both in the visible light range and in the infrared range, and feature values can be easily compared with each other.

When images of the same part are captured, regions of interest (ROIs) of the same size are set for the visible light imaging unit 122 and the infrared imaging unit 124. It may then be determined whether images of the same part have been captured by comparing images in the ROIs captured by the visible light imaging unit 122 and the infrared imaging unit 124 with each other through, for example, pattern recognition. In addition, the part may be identified by performing face recognition on the visible light image captured by the visible light imaging unit 122 and the infrared image captured by the infrared imaging unit 124, obtaining coordinates and sizes of feature points on the user's eyes, nose, and mouth, and calculating coordinates (relative positions) of the feature points on the user's eyes, nose, and mouth in consideration of a general ratio of sizes of the eyes, nose, and mouth.

As with skin images captured by the visible light imaging unit 122, skin images captured by the infrared imaging unit 124 are images of the same part of a person's skin including the person's face or hand captured at a plurality of temporally successive timings and are a moving image or a plurality of still images. The infrared imaging unit 124 is achieved, for example, by the infrared camera 24.

The pulse wave calculation device 100 includes the visible light waveform calculation unit 111, the infrared waveform calculation unit 112, a correlation degree calculation unit 113, a control pattern obtaining unit 114, the light source control unit 115, and a biological information calculation unit 116. The components of the pulse wave calculation device 100 will be described hereinafter.

### Visible Light Waveform Calculation Unit

The visible light waveform calculation unit 111 obtains visible light images from the visible light imaging unit 122 and extracts a visible light waveform, which indicates the user's pulse waves, from the obtained visible light images. The visible light waveform calculation unit 111 extracts a first visible light waveform from first visible light images obtained before the amount of light of the lighting device 30 is adjusted. In addition, the visible light waveform calculation unit 111 extracts a second visible light waveform from second visible light images obtained after the amount of light of the lighting device 30 is adjusted. When the amount of light of the lighting device 30 is adjusted, the light source control unit 115, which will be described later, outputs a visible light control signal for increasing or decreasing the amount of visible light of the lighting device 30 to the lighting device 30. A plurality of visible light images obtained from the visible light imaging unit 122 thus include the first visible light images obtained before the amount of light of the lighting device 30 is adjusted and the second visible light images obtained after the amount of light of the lighting device 30 is adjusted. Visible light waveforms extracted from the plurality of visible light images include the first visible light waveform extracted from the first visible light images and the second visible light waveform extracted from the second visible light images.

The visible light waveform calculation unit 111 may extract a plurality of first feature points, which are certain feature points of the extracted first visible light waveform. More specifically, the visible light waveform calculation unit 111 divides the first visible light waveform into a plurality of first unit waveforms in accordance with pulse wave period units, which are periods of pulse waves. The visible light waveform calculation unit 111 then extracts a plurality of first peaks from the first visible light waveform by extracting, from each of the plurality of first unit waveforms, a first peak, which is either a first top point that is a maximum value of the first unit waveform or a first bottom point that is a minimum value of the first unit waveform. The first peaks are an example of the first feature points.

The visible light waveform calculation unit 111 obtains timings of pulse waves as feature points of a visible light waveform and calculates heartbeat intervals from the timings of adjacent pulse waves. That is, the visible light waveform calculation unit 111 calculates a period from each of the plurality of extracted first feature points to an adjacent first feature point as a first heartbeat interval. For example, the visible light waveform calculation unit 111 calculates a plurality of first heartbeat intervals, each of which is a period from a first time point, at which one of the plurality of extracted first peaks occurs, to a second time point, at which a first peak temporally adjacent to the foregoing first peak occurs.

More specifically, the visible light waveform calculation unit 111 extracts a visible light waveform on the basis of temporal changes in luminance extracted from a plurality of visible light images associated with timings at which the plurality of visible light images have been captured. That is, the plurality of visible light images obtained from the visible light imaging unit 122 are associated with time points at which the visible light imaging unit 122 has captured the plurality of visible light images. The visible light waveform calculation unit 111 obtains timings of the user's pulse waves (hereinafter referred to as “pulse wave timings”) by obtaining intervals of certain feature points of the visible light waveform. The visible light waveform calculation unit 111 then calculates a period from each of the plurality of obtained pulse wave timings to a next pulse wave timing as a heartbeat interval.

In addition, the visible light waveform calculation unit 111 may extract a plurality of third feature points, which are certain feature points of the extracted second visible light waveform. More specifically, the visible light waveform calculation unit 111 may divide the second visible light waveform into a plurality of third unit waveforms in accordance with pulse wave period units. The visible light waveform calculation unit 111 may then extract a plurality of third peaks from the second visible light waveform by extracting, from each of the plurality of third unit waveforms, a third peak, which is either a third top point that is a maximum value of the third unit waveform or a third bottom point that is a minimum value of the third unit waveform. The third peaks are an example of the third feature points.

The visible light waveform calculation unit 111 may calculate a plurality of third heartbeat intervals, each of which is a period from a fifth time point, at which one of the plurality of extracted third peaks occurs, to a sixth time point, at which a third peak temporally adjacent to the foregoing third peak occurs.

For example, the visible light waveform calculation unit 111 identifies, using an extracted visible light waveform, a timing at which a largest change in luminance occurs as a pulse wave timing. Alternatively, the visible light waveform calculation unit 111 identifies positions of the user's face or hand in a plurality of visible light images using face or hand patterns stored in advance and then identifies a visible light waveform on the basis of temporal changes in luminance at the identified position. The visible light waveform calculation unit 111 calculates pulse wave timings using the identified visible light waveform. Here, the pulse wave timings are time points of certain feature points of a time waveform of luminance, that is, a time waveform of pulse waves. The certain feature points are, for example, peaks (top or bottom points) of the time waveform of luminance. The visible light waveform calculation unit 111 can identify the peaks, for example, using one of known local search methods including hill climbing, autocorrelation, and a method employing a differential function. The visible light waveform calculation unit 111 is achieved, for example, by the CPU 101, the main memory 102, and the storage 103.

Pulse waves are generally changes in blood pressure or volume in peripheral blood vessels according to heartbeats. That is, pulse waves are changes in the volume of blood vessels at a time when blood fed from the heart reaches to the face or the hands. When the volume of blood vessels in the face or the hands changes, the amount of blood flowing through the blood vessels changes, and the color of the skin changes depending on the amount of components of blood, such as hemoglobin. The luminance of the face or the hands in captured images, therefore, changes in accordance with pulse waves. That is, if temporal changes in the luminance of the face or a hand obtained from images of the face or the hand captured at a plurality of timings are used, information regarding the movement of blood can be obtained. The visible light waveform calculation unit 111 thus obtains pulse wave timings by calculating information regarding the movement of blood from a plurality of images captured over time.

When pulse wave timings are obtained in the visible light range, parts of visible light images including luminance in a wavelength range of green may be used. This is because changes caused by pulse waves are evident at the luminance in the wavelength range of green in images captured in the visible light range. In a visible light image including a plurality of pixels, the luminance in the wavelength range of green at pixels corresponding to the face or a hand to which a large amount of blood is flowing is lower than the luminance in the wavelength range of green at pixels corresponding to the face or a hand to which a small amount of blood is flowing.

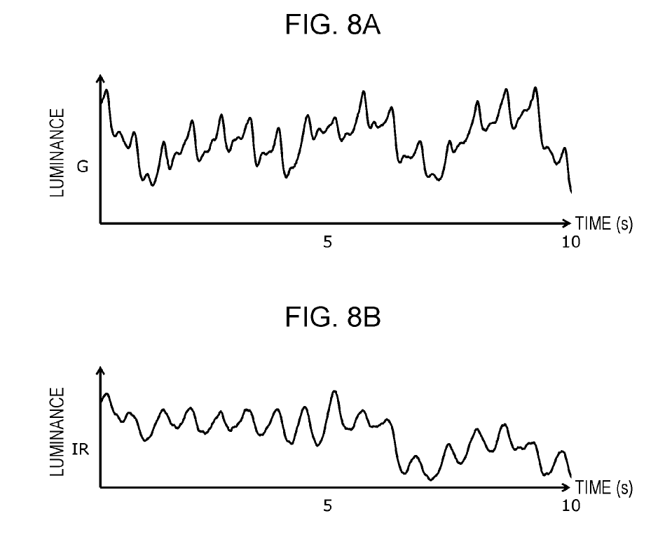

FIG. 8A is a graph illustrating an example of changes in luminance in visible light images, especially changes in the luminance in the wavelength range of green, according to the present embodiment. More specifically, FIG. 8A illustrates changes in the luminance of a green component (G) in the user's cheeks in visible light images captured by the visible light imaging unit 122. In the graph of FIG. 8A, a horizontal axis represents time, and a vertical axis represents the luminance of the green component (G). The changes in luminance illustrated in FIG. 8A indicate that the luminance periodically changes in accordance with pulse waves.

When images of the user's skin are captured in a usual environment, that is, in the visible light range, the visible light images include noise due to various factors including scattered light from the lighting device 30. The visible light waveform calculation unit 111 may therefore perform signal processing on visible light images obtained from the visible light imaging unit 122 using a filter or the like to obtain visible light images including more changes in the luminance of the user's skin due to pulse waves. The filter used for the signal processing may be, for example, a low-pass filter. That is, in the present embodiment, the visible light waveform calculation unit 111 extracts a visible light waveform through the low-pass filter on the basis of changes in the luminance of the green component (G).

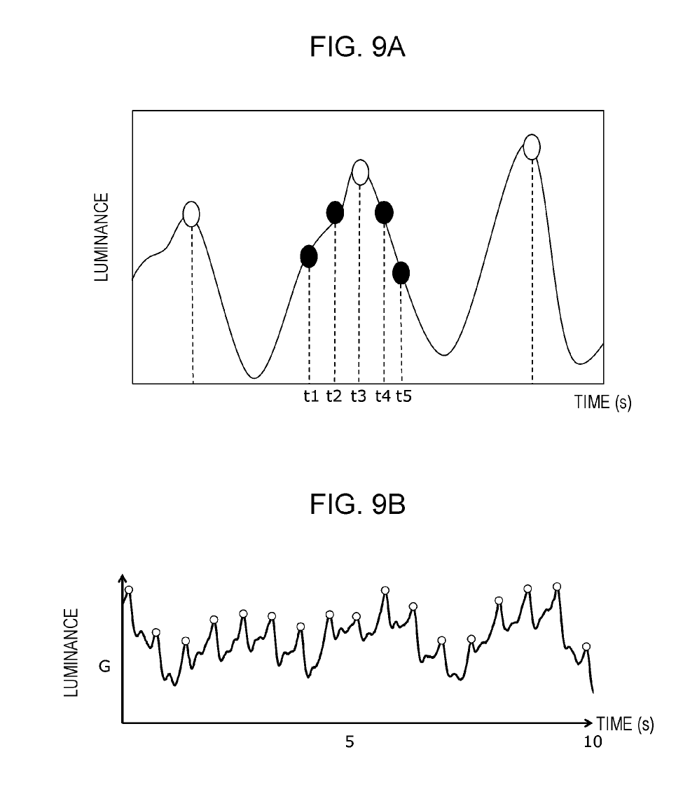

FIG. 9A is a graph illustrating an example of calculation of pulse wave timings according to the present embodiment. In the graph of FIG. 9A, a horizontal axis represents time, and a vertical axis represents luminance. In a time waveform illustrated in the graph of FIG. 9A, inflection points and a top point occur at time points t1 to t5. Points on the time waveform illustrated in the graph include inflection points and peaks (top or bottom points) as feature points. A top point refers to a maximum value of an upward wave in a time waveform, and a bottom point refers to a minimum value of a downward wave in a time waveform. Among such points on a time waveform, a point (top point) at which luminance is higher than at previous and next points or a point (bottom point) at which luminance is lower than at previous and next points is a pulse wave timing.

A method for identifying a top point, that is, a method for finding a peak, will be described with reference to the time waveform of luminance illustrated in the graph of FIG. 9A. The visible light waveform calculation unit 111 determines the point at the time point t2 on the time waveform of luminance as a current reference point. The visible light waveform calculation unit 111 compares the point at the time point t2 and the previous point at the time point t1 and compares the point at the time point t2 and the next point at the time t3. If a luminance at the reference point is higher than luminances at the previous and next points, the visible light waveform calculation unit 111 determines that a result is positive. That is, in this case, the visible light waveform calculation unit 111 determines that the reference point is a peak (top point) and the time point t2 is a pulse wave timing.

If the luminance at the reference point is lower than the luminances at the previous point and/or the next point, the visible light waveform calculation unit 111 determines that the result is negative. That is, in this case, the visible light waveform calculation unit 111 determines that the reference point is not a peak (top point) and that the time point t2 is not a pulse wave timing.

In FIG. 9A, the luminance at the time point t2 is higher than the luminance at the time point t1 but lower than the luminance at the time point t3. The visible light waveform calculation unit 111 therefore determines that the point at the time point t2 is not a peak. Next, the visible light waveform calculation unit 111 moves to a next reference point, that is, determines the point at the time point t3 as a reference point. Since the luminance at the time point t3 is higher than the luminance at the time point t2 and a luminance at the time point t4, the visible light waveform calculation unit 111 determines that the point at the time point t3 is a peak. The visible light waveform calculation unit 111 outputs time points determined as pulse wave timings to the correlation degree calculation unit 113. As a result, as illustrated in FIG. 9B, time points indicated by circles are identified as pulse wave timings.

Alternatively, the visible light waveform calculation unit 111 may identify pulse wave timings on the basis of knowledge about a normal heart rate (e.g., 60 to 180 bpm), that is, normal heartbeat intervals of 333 to 1,000 ms. When the normal heartbeat intervals are taken into consideration, the visible light waveform calculation unit 111 need not perform the above-described comparison of luminance for every point. In this case, the visible light waveform calculation unit 111 can identify appropriate pulse timings just by performing the comparison of luminance for some points. That is, the above-described comparison of luminance may be performed while using points located within a period of 333 to 1,000 ms since a latest pulse wave timing as reference points. In this case, a next pulse wave timing can be identified without performing the comparison of luminance while using earlier points as reference points. Robust pulse wave timings can therefore be obtained in a usual environment.

The visible light waveform calculation unit 111 also calculates heartbeat intervals by calculating time differences between adjacent pulse wave timings. The heartbeat interval varies over time. By comparing a heartbeat interval with a heartbeat interval based on pulse waves identified from an infrared waveform obtained in the same time period, a degree of correlation between certain feature points of the visible light waveform and certain feature points of the infrared waveform can be calculated.

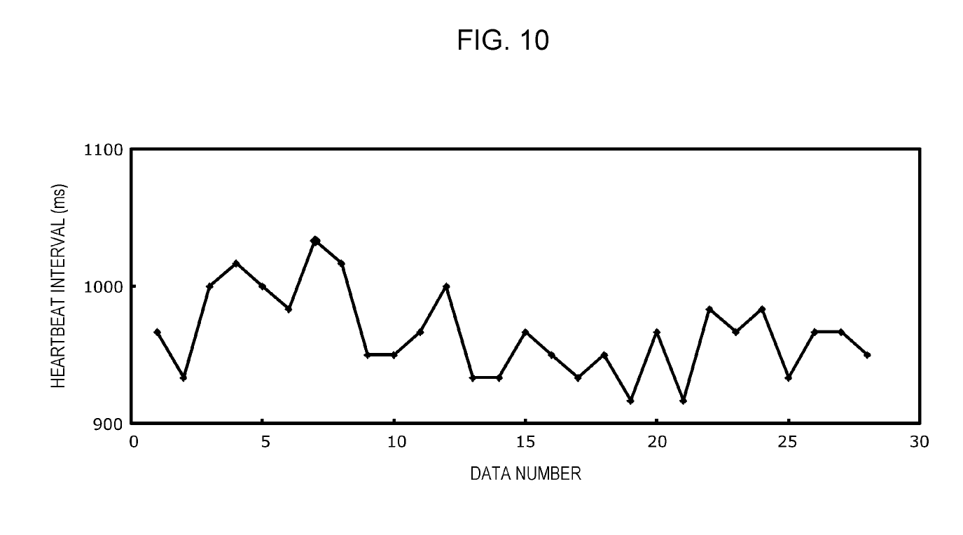

FIG. 10 is a graph illustrating an example of heartbeat intervals obtained over time. In the graph of FIG. 10, a horizontal axis represents data numbers associated with heartbeat intervals obtained over time, and a vertical axis represents heartbeat intervals. As illustrated in FIG. 10, the heartbeat interval varies over time. The data numbers refer to order in which data (heartbeat intervals here) is stored in a memory. That is, a data number corresponding to an n-th (n is a natural number) heartbeat interval stored in the memory is n.

The visible light waveform calculation unit 111 may also extract, from the visible light waveform, a time point of an inflection point immediately after each pulse wave timing. More specifically, the visible light waveform calculation unit 111 obtains a minimum point of visible light differential luminance by calculating first derivatives of luminance in the visible light waveform, and determines a time point of the minimum point as a time point of the inflection point (hereinafter referred to as an “inflection point timing”). That is, the visible light waveform calculation unit 111 may extract a plurality of inflection points between top points and bottom points as certain feature points.

The visible light waveform calculation unit 111 may calculate inflection point timings on the basis of knowledge about the normal heart rate, that is, the normal heartbeat intervals of 333 to 1,000 ms. In this case, even if a visible light waveform includes an inflection point that is not related to heartbeats, the inflection point is not identified. As a result, inflection point timings can be calculated more accurately.

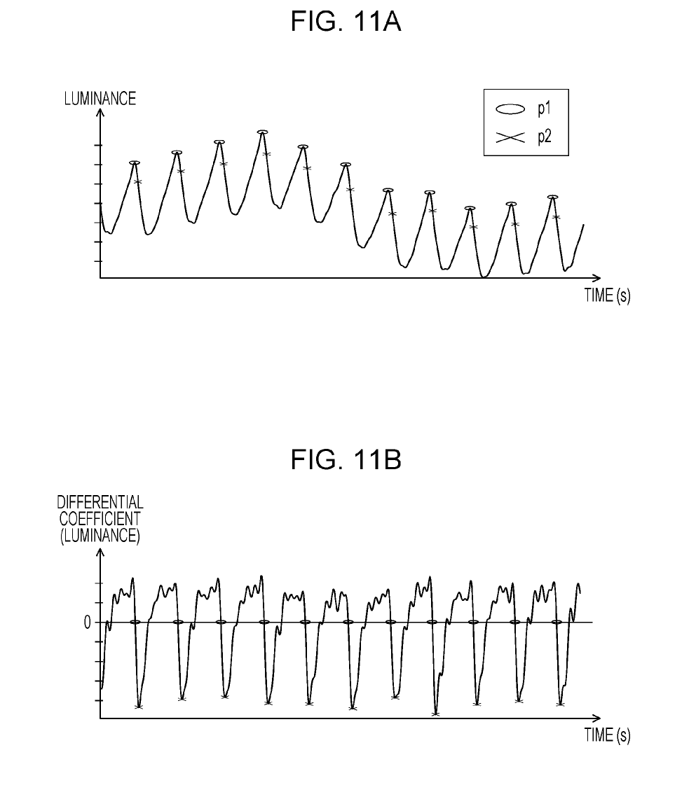

FIG. 11A and FIG. 11B are graphs illustrating a method for extracting inflection points from pulse waves. More specifically, FIG. 11A is a graph illustrating a visible light waveform obtained from visible light images, and FIG. 11B is a graph in which first derivatives of the visible light waveform illustrated in FIG. 11A are plotted. In FIG. 11A, circles indicate top points among peaks, and x's indicate inflection points. In FIG. 11B, circles indicate points corresponding to the top points illustrated in FIG. 11A, and x's indicate points corresponding to the inflection points illustrated in FIG. 11A. In the graph of FIG. 11A, a horizontal axis represents time, and a vertical axis represents luminance. In the graph of FIG. 11B, a horizontal axis represents time, and a vertical axis represents a differential coefficient of luminance.

As described above, when a visible light waveform is extracted, visible light images mainly including green light is used. How such a visible light waveform is extracted will be described hereinafter. When the amount of blood in blood vessels of the face or a hand increases or decreases in accordance with pulse waves, the amount of hemoglobin in blood accordingly increases or decreases. That is, as the amount of blood in blood vessels increases or decreases, the amount of hemoglobin, which absorbs light in the wavelength range of green, increases or decreases. In visible light images captured by the visible light imaging unit 122, therefore, the color of the skin near blood vessels, especially the luminance of a green component in visible light, varies as the amount of blood increases or decreases. More specifically, since hemoglobin absorbs green light, luminance in visible light images accordingly decreases.

Furthermore, in a visible light waveform, a gradient from a top point to a bottom point is higher than a gradient from a bottom point to a top point. For this reason, the visible light waveform is relatively susceptible to noise between a bottom point and a next top point. Between a top point and a next bottom point, on the other hand, the visible light waveform is hardly affected by noise since the gradient is high. Inflection point timings between a top point and a next bottom point are also hardly affected by noise and can be relatively stably obtained. The visible light waveform calculation unit 111 may therefore calculate time differences between inflection points between top points and next bottom points as heartbeat intervals.

The peaks in the visible light waveform are points at which the differential coefficient becomes zero immediately before the inflection points. More specifically, as illustrated in FIG. 11B, time points of points at which the differential coefficient becomes zero immediately before the x's, which indicate the inflection points, are time points of the circles, which indicate the top points in FIG. 11A. In consideration of this characteristic, the visible light waveform calculation unit 111 may limit top points to be obtained from a visible light waveform to ones immediately before inflection points.

The visible light waveform calculation unit 111 also calculates gradients from top points to bottom points in the visible light waveform. The visible light waveform calculation unit 111 calculates a first gradient of a first line connecting each of a plurality of first top points and one of a plurality of first bottom points immediately after the first top point. The gradients in the visible light waveform are preferably set as high as possible by adjusting the luminance of the lighting device 30. This is because as the gradients become higher, the sharpness of the visible light waveform at top points becomes higher, and errors in pulse wave timings obtained through filtering or the like become smaller.

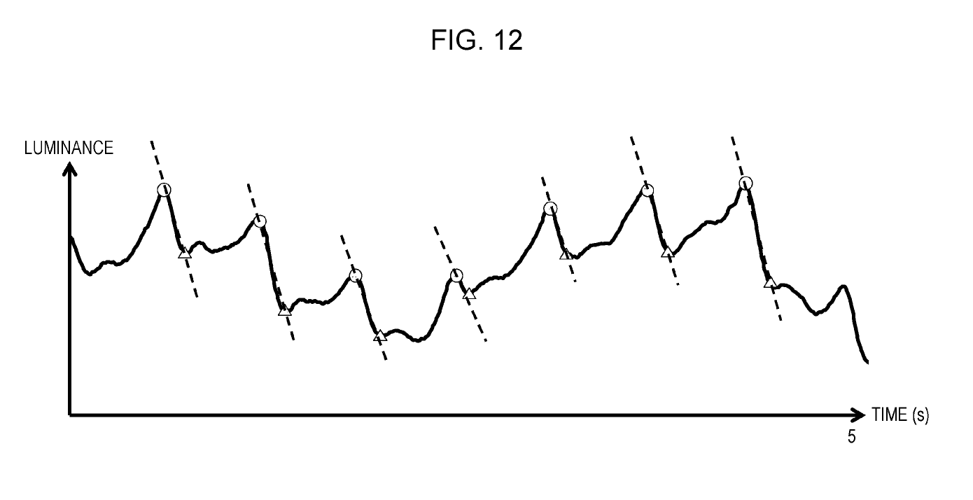

FIG. 12 is a graph illustrating a visible light waveform whose gradients are calculated. In the graph of FIG. 12, a horizontal axis represents time, a vertical axis represents luminance, circles indicate top points, and triangles indicate bottom points. The visible light waveform calculation unit 111 connects each top point (circle) and a next bottom point (triangle) with a straight line and calculates a gradient of the straight line. The calculated gradient differs depending on the amount of light of the lighting device 30, a part of the user's skin whose image is captured by the visible light imaging unit 122, and the like. The amount of light of the lighting device 30 and an ROI corresponding to the part of the user's skin whose image is captured by the visible light imaging unit 122 are set such that pulse waves are clearly obtained, that is, for example, the visible light waveform calculation unit 111 obtains pulse wave timings within the heartbeat intervals of 333 to 1,000 ms. The visible light waveform calculation unit 111 then records gradient information and compares the gradient information with gradient information based on pulse waves identified using infrared light. In an initial state, that is, after the lighting device 30 is turned on, the visible light waveform calculation unit 111 also records, in a memory (e.g., the storage 103) as a first gradient A, a gradient from a top point to a bottom point in the visible light waveform before the light source control unit 115 changes the amount of visible light of the lighting device 30 or the amount of infrared light of the infrared light source 123. The pulse wave measuring apparatus 10 compares feature points between the visible light waveform and an infrared waveform while gradually decreasing the amount of light of the lighting device 30 to zero and increasing the amount of the light of the infrared light source 123. Since the amount of visible light is gradually decreased, a gradient from a top point to a bottom point in the visible light waveform becomes highest in the initial state.

### Infrared Waveform Calculation Unit

The infrared waveform calculation unit 112 obtains infrared images from the infrared imaging unit 124 and extracts an infrared waveform, which indicates the user's pulse waves, from the obtained infrared images. The infrared waveform calculation unit 112 extracts a first infrared waveform from first infrared images obtained before the amount of light of the infrared light source 123 is adjusted. The infrared waveform calculation unit 112 extracts a second infrared waveform from second infrared images obtained after the amount of light of the infrared light source 123 is adjusted. When the amount of light of the infrared light source 123 is adjusted, the light source control unit 115, which will be described later, outputs an infrared control signal for increasing or decreasing the amount of infrared light of the infrared light source 123 to the infrared light source 123. A plurality of infrared images obtained from the infrared imaging unit 124 thus include the first infrared images obtained before the amount of light of the infrared light source 123 is adjusted and the second infrared images obtained after the amount of light of the infrared light source 123 is adjusted.

The infrared waveform calculation unit 112 may extract a plurality of second feature points, which are certain feature points of the extracted first infrared waveform. More specifically, the infrared waveform calculation unit 112 divides the first infrared waveform into a plurality of second unit waveforms in accordance with pulse wave period units. The infrared waveform calculation unit 112 then extracts a plurality of second peaks from the first infrared waveform by extracting, from each of the plurality of second unit waveforms, a second peak, which is either a second top point that is a maximum value of the second unit waveform or a second bottom point that is a minimum value of the second unit waveform. The second peaks are an example of the second feature points.

As with the visible light waveform calculation unit 111, the infrared waveform calculation unit 112 obtains timings of pulse waves as feature points of an infrared waveform and calculates heartbeat intervals from the timings of adjacent pulse waves. That is, the infrared waveform calculation unit 112 calculates a period from each of the plurality of extracted second feature points to an adjacent second feature point as a second heartbeat interval. More specifically, the infrared waveform calculation unit 112 extracts an infrared waveform on the basis of temporal changes in luminance extracted from a plurality of infrared images. That is, the plurality of infrared images obtained from the infrared imaging unit 124 are associated with time points at which the infrared imaging unit 124 has captured the infrared images. For example, the infrared waveform calculation unit 112 calculates a plurality of second heartbeat intervals, each of which is a period from a third time point, at which one of the plurality of extracted second peaks occurs, to a fourth time point, at which a second peak temporally adjacent to the foregoing second peak occurs.

The infrared waveform calculation unit 112 may extract a plurality of fourth feature points, which are certain feature points of the extracted second infrared waveform. More specifically, the infrared waveform calculation unit 112 may divide the second infrared waveform into a plurality of fourth unit waveforms in accordance with pulse wave period units. The infrared waveform calculation unit 112 may then extract a plurality of fourth peaks from the second infrared waveform by extracting, from each of the plurality of fourth unit waveforms, a fourth peak, which is either a fourth top point that is a maximum value of the fourth unit waveform or a fourth bottom point that is a minimum value of the fourth unit waveform. The fourth peaks are an example of the fourth feature points.

The infrared waveform calculation unit 112 may calculate a plurality of fourth heartbeat intervals, each of which is a period from a seventh time point, at which one of the plurality of extracted fourth peaks occurs, to an eighth time point, at which a fourth peak temporally adjacent to the foregoing fourth peak occurs.

As with the visible light waveform calculation unit 111, the infrared waveform calculation unit 112 can identify peaks, which are certain feature points of an infrared waveform, for example, using one of known local search methods including hill climbing, autocorrelation, and a method employing a differential function. As with the visible light waveform calculation unit 111, the infrared waveform calculation unit 112 is achieved, for example, by the CPU 101, the main memory 102, and the storage 103.

In an infrared image, as in a visible light image, the color of the skin, that is, the luminance of the face or a hand, generally changes depending on the amount of the compositions of blood such as hemoglobin. That is, if temporal changes in the luminance of the face or the hand obtained from images of the face or the hand captured at a plurality of timings are used, information regarding the movement of blood can be obtained. The infrared waveform calculation unit 112 thus obtains pulse wave timings by calculating information regarding the movement of blood from a plurality of images captured over time.

When pulse wave timings are obtained in the infrared range, parts of infrared images including luminance in a wavelength range of 800 nm and higher included in infrared images may be used. This is because changes caused by pulse waves are evident at the luminance in a wavelength range of 800 to 950 nm in images captured in the infrared range.

FIG. 8B is a graph illustrating an example of changes in luminance in infrared images according to the present embodiment. More specifically, FIG. 8B illustrates changes in the luminance of the user's cheeks in infrared images captured by the infrared imaging unit 124. In the graph of FIG. 8B, a horizontal axis represents time, and a vertical axis represents luminance. The changes in luminance illustrated in FIG. 8B indicate that the luminance periodically changes in accordance with pulse waves.

When images of the user's skin are captured in the infrared range, the amount of infrared light absorbed by hemoglobin is smaller than when images of the user's skin are captured in the visible light range. That is, due to various factors such as body movement, infrared images captured in the infrared range tend to include noise. Infrared images including more changes in the luminance of the user's skin caused by pulse waves, therefore, may be obtained by performing signal processing on the captured infrared images using a filter or the like and radiating an appropriate amount of infrared light onto the user's skin. The filter used for the signal processing may be, for example, a low-pass filter. That is, in the present embodiment, the infrared waveform calculation unit 112 extracts an infrared waveform through the low-pass filter on the basis of changes in the luminance of infrared light. A method for determining the amount of infrared light of the infrared light source 123 will be described later with reference to the correlation degree calculation unit 113 or the light source control unit 115.

Next, a method for finding a peak used by the infrared waveform calculation unit 112 will be described. The same method as the method for finding a peak in a visible light waveform can be used to find a peak in an infrared waveform.

As with the visible light waveform calculation unit 111, the infrared waveform calculation unit 112 may identify pulse wave timings on the basis of knowledge about the normal heart rate (e.g., 60 to 180 bpm), that is, the normal heartbeat intervals of 333 to 1,000 ms. When the normal heartbeat intervals are taken into consideration, the infrared waveform calculation unit 112 need not perform the above-described comparison of luminance for every point. In this case, the infrared waveform calculation unit 112 can identify appropriate pulse timings just by performing the comparison of luminance at some points. That is, the above-described comparison of luminance may be performed while using points located within a period of 333 to 1,000 ms since a latest pulse wave timing as reference points. In this case, a next pulse wave timing can be identified without performing the comparison of luminance while using earlier points as reference points.

As with the visible light waveform calculation unit 111, the infrared waveform calculation unit 112 also calculates heartbeat intervals by calculating time differences between adjacent pulse wave timings. The infrared waveform calculation unit 112 may also extract, from an infrared waveform, a time point of an inflection point immediately after each pulse wave timing. For example, the infrared waveform calculation unit 112 obtains a minimum point of infrared differential luminance by calculating first derivatives of luminance in the infrared waveform, and determines a time point of the minimum point as a time point of the inflection point (inflection point timing). That is, the infrared waveform calculation unit 112 may extract a plurality of inflection points between top points and bottom points as certain feature points.

In addition, as with the visible light waveform calculation unit 111, the infrared waveform calculation unit 112 calculates gradients from top points to bottom points in the infrared waveform. That is, the infrared waveform calculation unit 112 calculates, in the second infrared waveform, a second gradient of a second line connecting each of a plurality of fourth top points and one of a plurality of fourth bottom points immediately after the fourth top point.

As described above, by performing the same process as the visible light waveform calculation unit 111, the infrared waveform calculation unit 112 extracts a plurality of certain feature points as second feature points. Compared to a visible light waveform, however, an infrared waveform greatly varies depending on the amount of infrared light of a light source. That is, an infrared waveform is affected by the amount of light of the light source more easily than a visible light waveform.

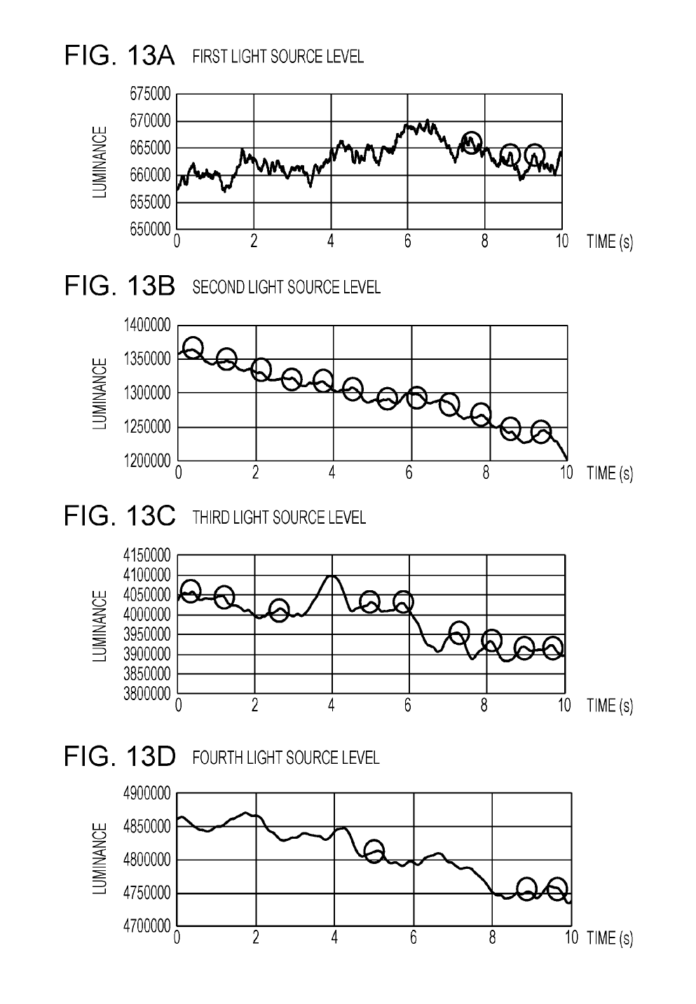

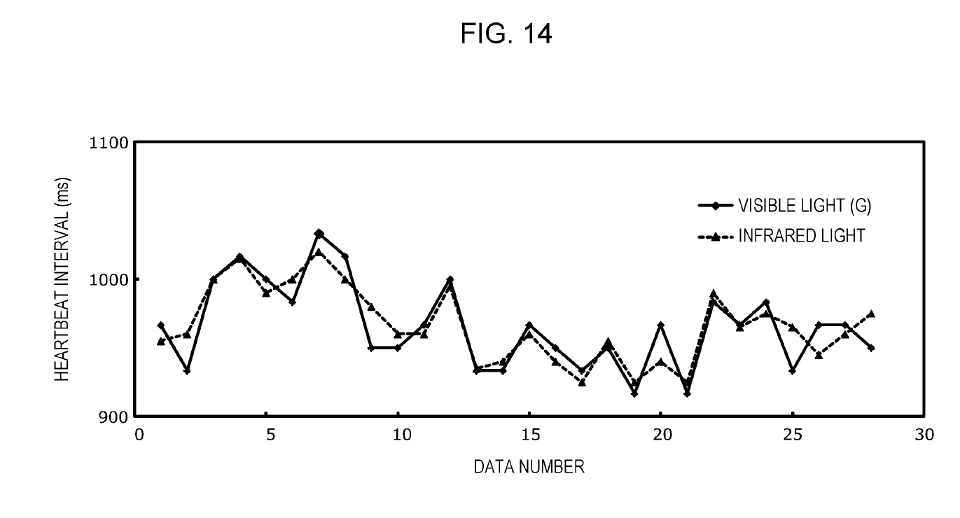

FIGS. 13A, 13B, 13C, and 13D are graphs illustrating infrared waveforms when an infrared camera has captured images of a person's skin with different amounts of light of an infrared light source. The amount of light of the infrared light source increases in order of FIGS. 13A to 13D. That is, a first light source level indicates a smallest amount of light, and a fourth light source level indicates a largest amount of light. A control voltage for the light source increases by about 0.5 V as a light source level increments. Circles in the graphs of FIG. 13A indicate peaks (top points) of pulse waves. As illustrated in FIGS. 13A, 13B, 13C, and 13D, when the amount of light of the light source is small, noise is larger than infrared light from the infrared light source, and it is difficult to identify pulse wave timings. As illustrated in FIGS. 13C and 13D, on the other hand, when the amount of light of the light source is large, changes in the luminance of the skin caused by pulse waves are buried under the amount of light and pulse waves become small. As a result, it is difficult to identify pulse wave timings.

When pulse waves are obtained using images captured in the visible light range by radiating visible light, the pulse waves can be stably obtained even if the amount of visible light is low enough not to hurt the user's eyes. When pulse waves are obtained using images captured in the infrared range by radiating infrared light, however, noise might be included or the amount of infrared light becomes too large as described above, even if the amount of infrared light is adjusted. For this reason, pulse waves can be obtained only within a strictly limited range of the amount of light. In addition, because an appropriate amount of light of the infrared light source varies depending on a part of the user's skin whose images are captured, the user's skin type, the color of the user's skin, and the like, it is difficult to set the amount of light to a certain value in advance. The correlation degree calculation unit 113, which will be described hereinafter, therefore, needs to perform an operation for adjusting the amount of infrared light to an appropriate value while decreasing the amount of visible light such that a visible light waveform and an infrared waveform match.

### Correlation Degree Calculation Unit

The correlation degree calculation unit 113 calculates a degree of correlation between a visible light waveform obtained from the visible light waveform calculation unit 111 and an infrared waveform obtained from the infrared waveform calculation unit 112. The correlation degree calculation unit 113 then determines instructions to adjust the amount of light of the lighting device 30 and the amount of light of the infrared light source 123 in accordance with the calculated degree of correlation, and transmits the determined instructions to the light source control unit 115.

The correlation degree calculation unit 113 obtains a plurality of first heartbeat intervals calculated from a first visible light waveform and a plurality of second heartbeat intervals calculated from a first infrared waveform from the visible light waveform calculation unit 111 and the infrared waveform calculation unit 112, respectively. The correlation degree calculation unit 113 then calculates a first degree of correlation between the plurality of first heartbeat intervals and the plurality of second heartbeat intervals temporally corresponding to each other.

The correlation degree calculation unit 113 also obtains a plurality of third heartbeat intervals calculated from a second visible light waveform and a plurality of fourth heartbeat intervals calculated from a second infrared waveform from the visible light waveform calculation unit 111 and the infrared waveform calculation unit 112, respectively. The correlation degree calculation unit 113 may then calculate a second degree of correlation between the plurality of third heartbeat intervals and the plurality of fourth heartbeat intervals temporally corresponding to each other.

FIG. 14 is a graph in which the first heartbeat intervals and the second heartbeat intervals are plotted in chronological order. In the graph of FIG. 14, a horizontal axis represents data numbers in chronological order, and a vertical axis represents heartbeat intervals corresponding to the data numbers. The data number refers to order in which data regarding the heartbeat intervals are stored in a memory. That is, a data number corresponding to an n-th (n is a natural number) first heartbeat interval stored in the memory is n. In addition, a data number corresponding to an n-th (n is a natural number) second heartbeat interval stored in the memory is n. Furthermore, since a first heartbeat interval and a second heartbeat interval are results obtained by measuring pulse waves occurring at the same timing, the first heartbeat interval and the second heartbeat interval are results obtained by measuring pulse waves at substantially the same timing insofar as data numbers are the same, unless there is no measurement error. That is, the plurality of first heartbeat intervals and the plurality of second heartbeat intervals include a combination of a first heartbeat interval and a second heartbeat interval temporally corresponding to each other.

The correlation degree calculation unit 113 calculates a degree of correlation between the plurality of first heartbeat intervals and the plurality of second heartbeat intervals using a correlation method. More specifically, the correlation degree calculation unit 113 calculates a first correlation coefficient between the plurality of first heartbeat intervals and the plurality of second heartbeat intervals temporally corresponding to each other as a first degree of correlation using the following expression (1).

$$ρ1 = \frac{{σ}_{12}}{{σ}_{1} {σ}_{2}} \qquad ( 1 )$$

ρ1: First correlation coefficient
σ₁₂: Covariance between plurality of first heartbeat intervals and plurality of second heartbeat intervals
σ₁: First standard deviation, standard deviation of plurality of first heartbeat intervals
σ₂: Second standard deviation, standard deviation of plurality of second heartbeat intervals

The correlation degree calculation unit 113 also calculates a second correlation coefficient between the plurality of third heartbeat intervals and the plurality of fourth heartbeat intervals temporally corresponding to each other as a second degree of correlation using the following expression (2).

$$ρ2 = \frac{{σ}_{34}}{{σ}_{3} {σ}_{4}} \qquad ( 2 )$$

ρ₂: Second correlation coefficient
σ₃₄: Covariance between plurality of third heartbeat intervals and plurality of fourth heartbeat intervals
σ₃: Third standard deviation, standard deviation of plurality of third heartbeat intervals
σ₄: Fourth standard deviation, standard deviation of plurality of fourth heartbeat intervals

If the first correlation coefficient is equal to or larger than a second threshold (certain threshold), namely 0.8, for example, the correlation degree calculation unit 113 determines that the plurality of first heartbeat intervals and the plurality of second heartbeat intervals substantially match. In this case, the correlation degree calculation unit 113 outputs a “true” signal, for example, to the light source control unit 115 as a signal indicating that the plurality of first heartbeat intervals and the plurality of second heartbeat intervals substantially match. If the first correlation coefficient is smaller than the second threshold, namely 0.8, for example, the correlation degree calculation unit 113 determines that the plurality of first heartbeat intervals and the plurality of second heartbeat intervals do not match. In this case, the correlation degree calculation unit 113 outputs a “false” signal, for example, to the light source control unit 115 as a signal indicating that the plurality of first heartbeat intervals and the plurality of second heartbeat intervals do not match. The correlation degree calculation unit 113 performs the above process on the second correlation coefficient as well as the first correlation coefficient.

In addition, the correlation degree calculation unit 113 may determine not only the degree of correlation between the plurality of first heartbeat intervals and the plurality of second heartbeat intervals but also whether these heartbeat intervals are appropriate and transmit a result of the determination to the light source control unit 115. More specifically, the correlation degree calculation unit 113 determines whether an absolute error between one of the plurality of first heartbeat intervals and one of the plurality of second heartbeat intervals corresponding to each other exceeds a third threshold (e.g., 200 ms). The correlation degree calculation unit 113 calculates an absolute error between a first heartbeat interval and a second heartbeat interval whose data numbers are the same, for example, and determines whether the absolute error exceeds the third threshold. If determining that the absolute error exceeds the third threshold, for example, the correlation degree calculation unit 113 determines that the number of peaks of either the visible light waveform or the infrared waveform is too large. The correlation degree calculation unit 113 then transmits a waveform whose number of peaks is too large (the visible light waveform or the infrared waveform) to the light source control unit 115. The absolute error can be obtained using the following expression (3).

e=RRI_{RGB}=RRI_{IR} (3)

In expression (3), e denotes the absolute error between the first heartbeat interval and the second heartbeat interval corresponding to each other, RRI_{RGB} denotes the first heartbeat interval, and RRI_{IR} denotes the second heartbeat interval.

If e is smaller than (−1× third threshold) (e.g., −200 ms), for example, the correlation degree calculation unit 113 determines that the number of peaks of the visible light waveform is too large. If e is larger than the third threshold (e.g., 200 ms), the correlation degree calculation unit 113 determines that the number of peaks of the infrared waveform is too large. The correlation degree calculation unit 113 then transmits, to the light source control unit 115 as a result of the determination, information indicating the waveform whose number of peaks is too large. It can thus be identified on the basis of an error between the heartbeat intervals corresponding to each other in the two waveforms that too many peaks have been obtained or peaks have not been successfully obtained in one of the two waveforms.

If determining that the absolute error between the first heartbeat interval and the second heartbeat interval corresponding to each other exceeds the third threshold, and if determining that too many peaks have been obtained in the visible light waveform, for example, the correlation degree calculation unit 113 transmits, to the light source control unit 115, a “false, RGB” signal indicating the result of the determination. If determining that the absolute error exceeds the third threshold, and if determining that too many peaks have been obtained in the infrared waveform, the correlation degree calculation unit 113 transmits, to the light source control unit 115, a “false, IR” signal indicating the result of the determination.

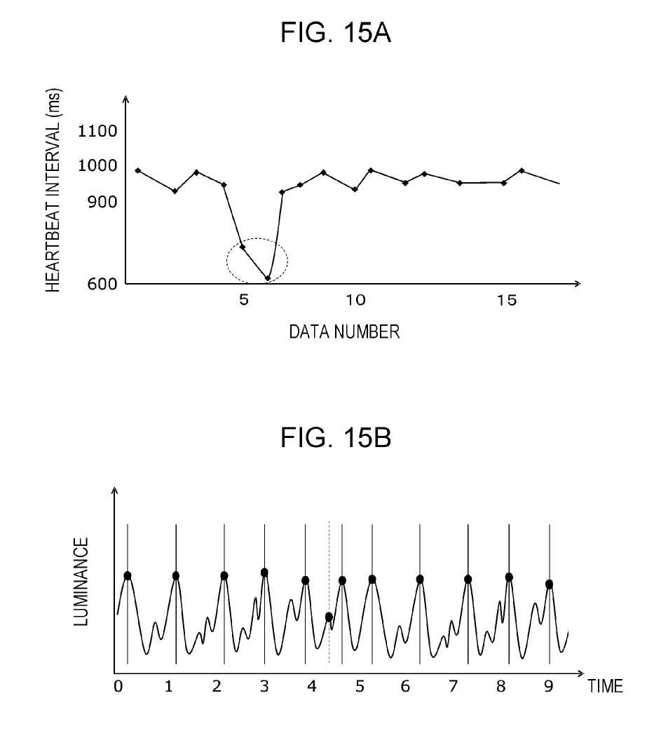

FIG. 15 is a diagram illustrating a specific example of a determination whether heartbeat intervals are appropriate. FIG. 15A is a graph illustrating a case in which a plurality of obtained heartbeat intervals is not appropriate. FIG. 15B is a graph illustrating an example of a visible light waveform or an infrared waveform corresponding to FIG. 15A. In the graph of FIG. 15A, a horizontal axis represents data numbers in chronological order, and a vertical axis represents heartbeat intervals corresponding to the data numbers. In the graph of FIG. 15B, a horizontal axis represents time, and a vertical axis represents luminance in images.

In FIG. 15A, a heartbeat interval between two points surrounded by a broken line is not appropriate. The heartbeat interval generally fluctuates, but usually does not sharply change. As illustrated in FIG. 15A, for example, an average of heartbeat intervals is about 950 ms and a standard deviation is about 50 ms outside the broken line. The heartbeat interval between the two points surrounded by the broken line, however, is about 600 to 700 ms because a point indicated by a broken line in FIG. 15B has been obtained as a peak. That is, the visible light waveform calculation unit 111 or the infrared waveform calculation unit 112 has obtained one too many peaks.

If the visible light waveform calculation unit 111 or the infrared waveform calculation unit 112 has obtained the result illustrated in FIGS. 15A and 15B, the number of pieces of data does not match between the plurality of first heartbeat intervals and the plurality of second heartbeat intervals.

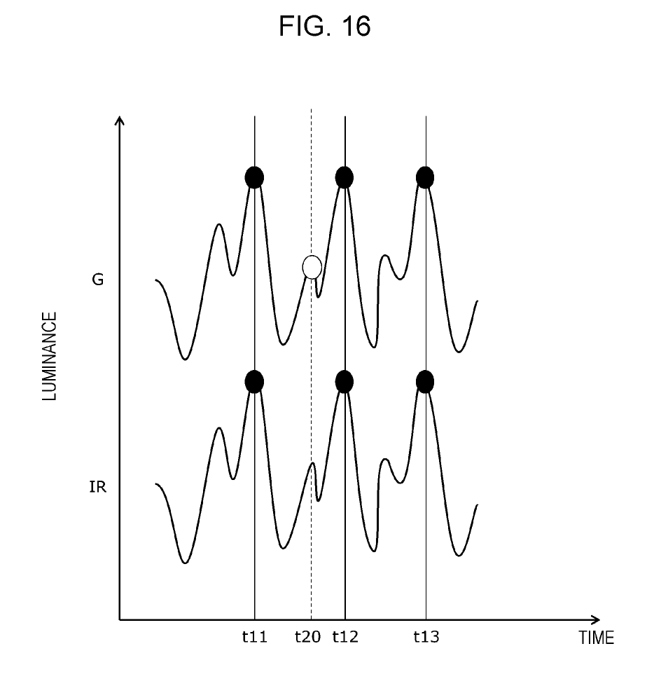

FIG. 16 illustrates details of this condition. FIG. 16 is a diagram illustrating an example of a case in which too many peaks have been obtained in a visible light waveform and too many peaks have not been obtained in a corresponding infrared waveform.

Data regarding a plurality of first or second heartbeat intervals is stored in the storage 103, for example, as combinations of a data number and a heartbeat interval. Data indicating a plurality of first heartbeat intervals obtained from the visible light waveform is, for example, (x, t20-t11), (x+1, t12-t20), and (x+2, t13-t12). Data indicating a plurality of second heartbeat intervals obtained from the infrared waveform is, for example, (x, t12-t11) and (x+1, t13-t12). The number of pieces of data is different between the visible light waveform and the infrared waveform although the data has been obtained in the same time period t11 to t13. As a result, all subsequent first heartbeat intervals and second heartbeat intervals do not correspond to each other correctly, and a degree of correlation between temporal changes of the heartbeat intervals decreases.

If an absolute error between a third heartbeat interval and a fourth heartbeat intervals, which have been obtained by the visible light waveform calculation unit 111 and the infrared waveform calculation unit 112, respectively, at each data number is equal to or larger than the third threshold, namely 200 ms, for example, the correlation degree calculation unit 113 removes a pulse wave peak from the waveform whose number of peaks is larger. The correlation degree calculation unit 113 then decreases, by one, data numbers subsequent to a data number corresponding to the removed peak.

If determining that too many peaks (that is, certain feature points) have been obtained as described above, the correlation degree calculation unit 113 may exclude, from targets of calculation of heartbeat intervals, a certain feature point that has served as a reference for calculating a heartbeat interval in the waveform (the visible light waveform or the infrared waveform) whose number of certain feature points is larger. That is, if e is smaller than (−1× third threshold), the correlation degree calculation unit 113 excludes, from targets of calculation of first heartbeat intervals, a peak that has served as a reference for calculating RRI_{RGB} used to calculate e. If e is larger than the third threshold, the correlation degree calculation unit 113 excludes, from targets of calculation of second heartbeat intervals, a peak that has served as a reference for calculating RRI_{IR} used to calculate e.

That is, the correlation degree calculation unit 113 determines whether an absolute error between one of the plurality of third heartbeat intervals and one of the plurality of fourth heartbeat intervals temporally corresponding to each other exceeds the third threshold. If determining that the absolute error exceeds the third threshold, the correlation degree calculation unit 113 compares the number of third peaks and the number of fourth peaks. The correlation degree calculation unit 113 then identifies, from between the third heartbeat interval and the fourth heartbeat interval with which the absolute error has exceeded the third threshold, a heartbeat interval calculated using an excessive peak. The correlation degree calculation unit 113 excludes, from targets of calculation of heartbeat intervals, the peak that has served as a reference for calculating the identified heartbeat interval.

Too many peaks are obtained when much noise is included in an obtained waveform (a visible light waveform or an infrared waveform). The correlation degree calculation unit 113, therefore, identifies the waveform whose number of peaks is larger. The correlation degree calculation unit 113 then generates a “false, RGB” signal, for example, and transmits the generated signal to the light source control unit 115. By receiving the “false, RGB” signal, the light source control unit 115 can learn that heartbeat intervals do not match between the visible light waveform and the infrared waveform and that the visible light waveform is the culprit of the mismatch. Since an error in data regarding peaks of a visible light waveform and an infrared waveform can be identified and information indicating the identified error can be transmitted to the light source control unit 115, the user's pulse waves in the visible light waveform and the infrared waveform can be obtained more accurately.

Although the second threshold used by the correlation degree calculation unit 113 to determine the degree of correlation between the first heartbeat intervals and the second heartbeat intervals is 0.8, the second threshold is not limited to this. More specifically, the second threshold may be determined in accordance with the required accuracy of biological information to be measured by the user. If the user desires to more accurately obtain biological information during sleep, that is, information regarding a heart rate or blood pressure, by strictly extracting pulse waves during sleep using infrared light, for example, the second threshold may be larger, namely, for example, 0.9.

If the second threshold for the correlation coefficient that serves as a reference has been adjusted, the reliability of obtained data may be displayed on a display device 40 in accordance with the adjusted second threshold. When it is difficult to match feature values between a visible light waveform and an infrared waveform and reduce the amount of light of a visible light source during sleep, for example, the second threshold for the correlation coefficient that serves as a reference may be changed to a value smaller than 0.8, namely, for example, 0.6. In this case, the accuracy relating to the degree of correlation becomes lower, and the display device 40 may indicate that the reliability has decreased.

If the correlation coefficient between first and second heartbeat intervals obtained over time from a visible light waveform and an infrared waveform, respectively, is smaller than the second threshold, or if visible light waveform calculation unit 111 or the infrared waveform calculation unit 112 has obtained too many peaks in a first certain time period, the correlation degree calculation unit 113 may measure a degree of correlation between the visible light waveform and the infrared waveform using inflection points in the visible light waveform and the infrared waveform. That is, the correlation degree calculation unit 113 may calculate a correlation coefficient between a plurality of third heartbeat intervals calculated using first inflection points and a plurality of fourth heartbeat intervals calculated using second inflection points temporally corresponding to each other as the second correlation coefficient using expression (2).

More specifically, as described above, if a correlation coefficient between first and second heartbeat intervals in a visible light waveform and an infrared waveform is smaller than the second threshold, namely 0.8, or if the number of peaks obtained by the visible light waveform calculation unit 111 and the infrared waveform calculation unit 112 does not match in the first certain time period (e.g., five seconds) and the number of peaks of at least one of the two waveforms exceeds a first threshold (e.g., 10), for example, the correlation degree calculation unit 113 may use inflection points in the visible light waveform and the infrared waveform to determine a degree of correlation of interval information between the inflection points in the waveforms.

That is, the correlation degree calculation unit 113 makes a tenth determination for determining whether the number of third peaks or the number of fourth peaks exceeds the first threshold in the first certain time period. If determining that the number of third peaks or the number of fourth peaks exceeds the first threshold in the first certain time period, the correlation degree calculation unit 113 may perform the following process.

That is, the correlation degree calculation unit 113 causes the visible light waveform calculation unit 111 to extract a plurality of first inflection points, each of which is an inflection point between one of a plurality of third top points and one of a plurality of third bottom points immediately after the third top point. The correlation degree calculation unit 113 also causes the infrared waveform calculation unit 112 to extract a plurality of second inflection points, each of which is an inflection point between one of a plurality of fourth top points and one of a plurality of fourth bottom points immediately after the fourth top point. In addition, the correlation degree calculation unit 113 causes the visible light waveform calculation unit 111 to calculate, for each of the plurality of extracted first inflection points, an interval between a ninth time point of the first inflection point and a tenth time point of an adjacent first inflection point as a third heartbeat interval. The correlation degree calculation unit 113 also causes the infrared waveform calculation unit 112 to calculate, for each of the plurality of extracted second inflection points, an interval between a seventh time point of the second inflection point and an eighth time point of an adjacent second inflection point as a fourth heartbeat interval. The correlation degree calculation unit 113 then calculates a second correlation coefficient between the plurality of third heartbeat intervals calculated using the first inflection points and the plurality of fourth heartbeat intervals calculated using the second inflection points temporally corresponding to each other as the second degree of correlation using expression (2).

Alternatively, the correlation degree calculation unit 113 may calculate the second correlation coefficient between the plurality of third heartbeat intervals calculated using the first inflection points and the plurality of fourth heartbeat intervals calculated using the second inflection points temporally corresponding to each other as the second degree of correlation using expression (2) regardless of the result of the tenth determination in the following case: a case in which a standard deviation of heartbeat intervals calculated using peaks whose number is determined to be smaller as a result of comparison is equal to or smaller than a fourth threshold.

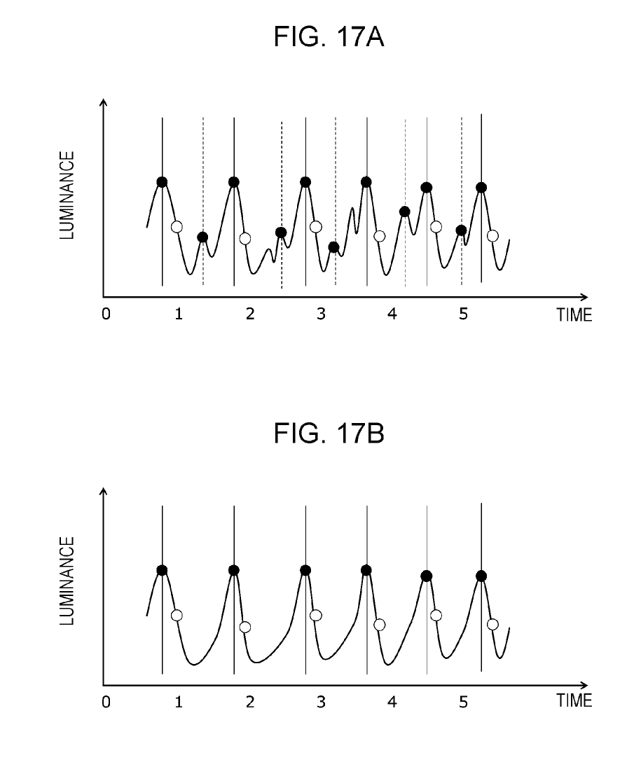

FIGS. 17A and 17B are diagrams illustrating a case in which the degree of correlation is calculated using inflection points. FIG. 17A is a graph illustrating peaks (top points) obtained from a visible light waveform, and FIG. 17B is a graph illustrating peaks (top points) obtained from an infrared waveform. In FIGS. 17A and 17B, horizontal axes represent time, vertical axes represent luminance, solid circles indicate obtained top points, and hollow circles indicate obtained inflection points.

In FIG. 17A, too many peaks have been obtained from the visible light waveform. During the first certain time period (five seconds), there are 10 or 11 peaks, which are equal to or larger than the first threshold. In FIG. 17B, on the other hand, peaks have been obtained from the infrared waveform at constant heartbeat intervals, and a standard deviation is 100 ms or smaller. At this time, chronological data numbers indicating first and second heartbeat intervals in the visible light waveform and the infrared waveform, respectively, do not match.

The correlation degree calculation unit 113 may therefore calculate a degree of correlation between the visible light waveform and the infrared waveform using the inflection points, which have been obtained by the visible light waveform calculation unit 111 and the infrared waveform calculation unit 112, included between top points and bottom points of pulse waves. For example, the correlation degree calculation unit 113 calculates the degree of correlation between the first and second heartbeat intervals by causing the visible light waveform calculation unit 111 and the infrared waveform calculation unit 112 to calculate the first and second heartbeat intervals, respectively, using the inflections points. More specifically, the correlation degree calculation unit 113 calculates the degree of correlation on the basis of correlation or an absolute error between the heartbeat intervals based on the inflection points in the visible light waveform and the infrared waveform.

Although the correlation degree calculation unit 113 calculates a degree of correlation between a visible light waveform and an infrared waveform using heartbeat intervals between inflection points if a correlation coefficient between heartbeat intervals in the visible light waveform or the infrared waveform is smaller than the second threshold or if the number of peaks of at least one of the two waveforms is larger than the first threshold in the first certain time period, the operation performed by the correlation degree calculation unit 113 is not limited to this. For example, the correlation degree calculation unit 113 may calculate a degree of correlation between a visible light waveform and an infrared waveform using not peaks but heartbeat intervals based on inflection points from a beginning, instead. In this case, the correlation degree calculation unit 113 can calculate intervals similar to heartbeat intervals by calculating heartbeat intervals based on inflection points even if it is difficult to accurately obtain peaks from a visible light waveform or an infrared waveform. Compared to heartbeat intervals obtained from peaks, heartbeat intervals based on inflection points do not include much noise but easily change positions thereof between top points and bottom points. That is, heartbeat intervals between top points are stable, a standard deviation thereof is usually within 100 ms, and temporal errors are smaller than in heartbeat intervals based on inflection points. In the present disclosure, therefore, heartbeat intervals calculated from peaks are used unless otherwise noted.

In addition, if the following condition is satisfied, the correlation degree calculation unit 113 may use heartbeat intervals based on inflection points to calculate a degree of correlation, instead of heartbeat intervals calculated from peaks. The condition is, for example, that a standard deviation of a plurality of heartbeat intervals corresponding to a visible light waveform or an infrared waveform whose number of peaks is smaller is equal to or smaller than the fourth threshold (e.g., 100 ms). The method in which whether too many peaks have been obtained is determined on the basis of the number of peaks in the first certain time period can be sometimes troublesome, because when the number of peaks in the first certain time period does not exceed the first threshold, peaks that are actually excessive might be overlooked.

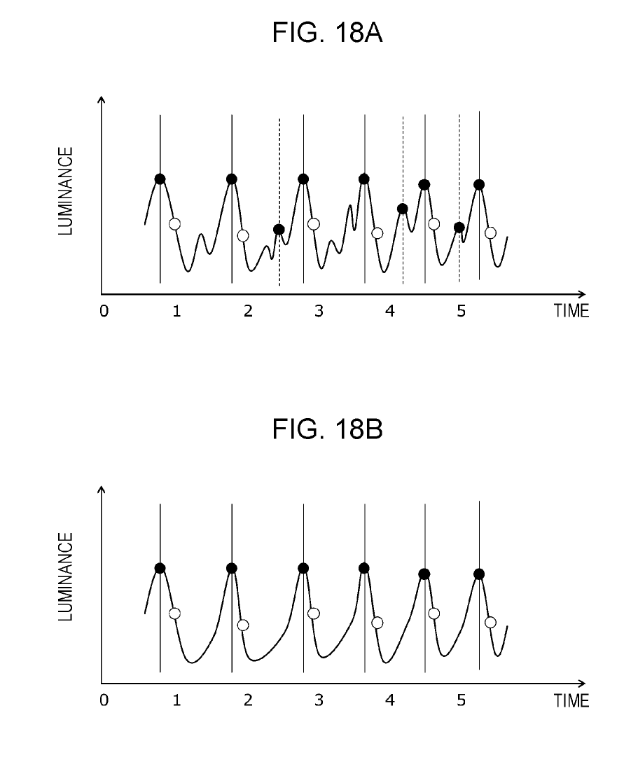

For example, FIG. 18A and FIG. 18B are diagrams illustrating an example in which there are too many peaks but the number of peaks in the first certain time period does not exceed the first threshold. In FIGS. 18A and 18B, horizontal axes represent time, vertical axes represent luminance, solid circles indicate obtained top points, and hollow circles indicate obtained inflection points.

As illustrated in FIG. 18A, if eight peaks have been obtained in five seconds in a visible light waveform, the number of peaks in the first certain time period does not exceed the first threshold, but the number of peaks obtained is different from the number of peaks obtained in an infrared waveform illustrated in FIG. 18B. As described above, if even one too many peaks are obtained, data numbers of first heartbeat intervals and second heartbeat intervals do not correspond to each other. If it can be proved that heartbeat intervals are substantially constant in either the visible light waveform or the infrared waveform, therefore, peaks can be adjusted (removed) in accordance with the number of peaks of the waveform. Details of the adjustment of peaks have been described with reference to FIG. 16.

If a standard deviation of heartbeat intervals in the first certain time period exceeds the fourth threshold in both a visible light waveform and an infrared waveform, the correlation degree calculation unit 113 determines that it is difficult to obtain appropriate pulse wave timings from the two waveforms, and transmits a “false, both” signal, which indicates that it is difficult to obtain appropriate pulse wave timings from the two waveforms, to the light source control unit 115.

If the visible light waveform calculation unit 111 appropriately obtains peaks in the first certain time period (that is, if a standard deviation of heartbeat intervals is smaller than the fourth threshold) after the pulse wave measuring apparatus 10 begins to operate, the correlation degree calculation unit 113 stores a gradient from a top point to a bottom point in the visible light waveform obtained by the visible light waveform calculation unit 111 in a memory as a first gradient A. Each time the light source control unit 115 has changed the amount of light of the lighting device 30 or the infrared light source 123, the correlation degree calculation unit 113 transmits an instruction to the light source control unit 115 such that a second gradient from a top point to a bottom point in the infrared waveform becomes the first gradient A. The correlation degree calculation unit 113 need not use peaks obtained while the light source control unit 115 is adjusting the amount of light of a light source for the calculation of a degree of correlation between a visible light waveform and an infrared waveform.

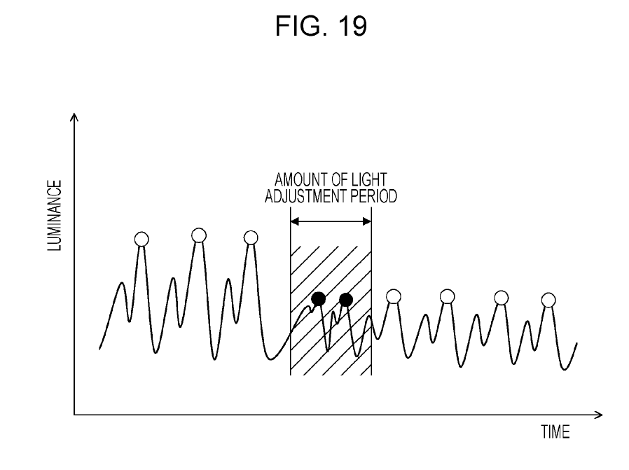

FIG. 19 is a graph illustrating an example in which peaks obtained while the amount of light of a light source is being adjusted are not used for the calculation of a degree of correlation between a visible light waveform and an infrared waveform. In the graph of FIG. 19, a horizontal axis represents time, a vertical axis represents luminance. The amount of light of the light source is adjusted in a hatched period. Hollow and solid circles indicate obtained peaks.

As illustrated in FIG. 19, when the amount of light of the light source is adjusted, gain in the luminance of the visible light waveform or the infrared waveform changes, and the sharpness at peaks accordingly changes. If the visible light waveform calculation unit 111 or the infrared waveform calculation unit 112 uses a filter for the peaks whose sharpness has changed, positions of the peaks move forward or backward along a time axis depending on the sharpness of the peaks in the original waveform. These errors do not pose a problem when a heart rate is calculated as biological information, but when blood pressure is calculated from pulse wave velocity, for example, these errors significantly affect a result. The pulse wave measuring apparatus 10 in the present disclosure, therefore, need not extract, from a visible light waveform or an infrared waveform, certain feature points (i.e., peaks) obtained while the amount of light of the lighting device 30 or the infrared light source 123 is being adjusted using first to fourth control signals.

That is, the visible light waveform calculation unit 111 extracts a plurality of first peaks from a first visible light waveform obtained in periods other than a period in which the amount of light of the lighting device 30 is being adjusted using a visible light control signal. In addition, the visible light waveform calculation unit 111 extracts a plurality of third peaks from a second visible light waveform obtained in periods other than a period in which the amount of light of the lighting device 30 is being adjusted using a third control signal.

The infrared waveform calculation unit 112 extracts a plurality of second peaks from a first infrared waveform obtained in periods other than a period in which the amount of light of the infrared light source 123 is being adjusted using an infrared control signal. In addition, the infrared waveform calculation unit 112 extracts a plurality of fourth peaks from a second infrared waveform obtained in periods other than a period in which the amount of the light of the infrared light source 123 is being adjusted using a fourth control signal.

Although if a correlation coefficient between heartbeat intervals in a visible light waveform and heartbeat intervals in an infrared waveform is smaller than the second threshold, the correlation degree calculation unit 113 determines that the number of peaks is excessive in either one or both of the two waveforms, calculates an error between the heartbeat intervals and standard deviations of the heartbeat intervals, and, if a certain condition is satisfied, uses heartbeat intervals based on inflections located between top points and bottom points of the waveforms, the operation performed by the correlation degree calculation unit 113 is not limited to this. If a correlation coefficient between first heartbeat intervals and second heartbeat intervals is smaller than the second threshold but peaks have been appropriately obtained in both waveforms (e.g., standard deviations of the heartbeat intervals in the two waveforms are both equal to or smaller than the fourth threshold), for example, the correlation degree calculation unit 113 transmits a “false” signal to the light source control unit 115.

The correlation degree calculation unit 113 thus transmits, to the light source control unit 115, a signal according to a calculated degree of correlation and a result of extraction of certain feature points from a visible light waveform and an infrared waveform (e.g., a “true”, “false”, “false, RGB”, “false, IR”, or “false, both” signal).

As described above, the correlation degree calculation unit 113 makes the following determinations on the basis of first heartbeat intervals and second heartbeat intervals.

That is, the correlation degree calculation unit 113 makes a second determination for determining whether a first standard deviation exceeds the fourth threshold and whether a second standard deviation exceeds the fourth threshold. If determining as a result of the second determination that the first standard deviation exceeds the fourth threshold and that the second standard deviation exceeds the fourth threshold, the correlation degree calculation unit 113 makes a third determination for determining whether a first time difference between one of a plurality of first heartbeat intervals and one of a plurality of second heartbeat intervals temporally corresponding to the first heartbeat interval is smaller than a fifth threshold and a fourth determination for determining whether the first time difference is larger than a sixth threshold, which is larger than the fifth threshold.

If determining as a result of the third and fourth determinations that the first time difference is smaller than the fifth threshold, the correlation degree calculation unit 113 makes a fifth determination for determining whether a second standard deviation is equal to or smaller than the fourth threshold.

In addition, the correlation degree calculation unit 113 may make the following determinations on the basis of third heartbeat intervals and fourth heartbeat intervals.

That is, the correlation degree calculation unit 113 makes a sixth determination for determining whether a third standard deviation exceeds the fourth threshold and whether a fourth standard deviation exceeds the fourth threshold. If determining as a result of the sixth determination that the third standard deviation exceeds the fourth threshold and that the fourth standard deviation exceeds the fourth threshold, the correlation degree calculation unit 113 makes a seventh determination for determining whether a second time difference between one of a plurality of third heartbeat intervals and one of a plurality of fourth heartbeat intervals temporally corresponding to the third heartbeat interval is smaller than the fifth threshold and an eighth determination for determining whether the second time difference is larger than the sixth threshold.

If determining as a result of the seventh and eighth determinations that the second time difference is smaller than the fifth threshold, the correlation degree calculation unit 113 makes a ninth determination for determining whether the fourth standard deviation is equal to or smaller than the fourth threshold.

### Control Pattern Obtaining Unit

The control pattern obtaining unit 114 obtains a control pattern predetermined in the lighting device 30 for adjusting the amount of light of the lighting device 30 arranged outside the pulse wave measuring apparatus 10. The control pattern obtaining unit 114 transmits the obtained control pattern to the light source control unit 115. More specifically, the control pattern obtaining unit 114 stores a plurality of control patterns for various models of the lighting device 30. Each time the lighting device 30 is identified, the control pattern obtaining unit 114 matches the plurality of control patterns stored therein and the identified lighting device 30 and selects a control pattern for controlling the identified lighting device 30.

The control pattern obtaining unit 114 may store, for example, product numbers used by various manufacturers and control patterns for controlling the lighting devices corresponding to the product numbers. In this case, when the user uses the pulse wave measuring apparatus 10 for the first time, for example, the control pattern obtaining unit 114 may receive a product number of the lighting device 30 and select a control pattern corresponding to the received product number. The user may input a product number through the pulse wave measuring apparatus 10 if the pulse wave measuring apparatus 10 includes an input interface such as input buttons or through a remote control application activated on the mobile terminal 200. In the latter case, the pulse wave measuring apparatus 10 receives a product number input to the mobile terminal 200 from the mobile terminal 200. As a result, the control pattern obtaining unit 114 can recognize a control pattern corresponding to each product number and select a control signal corresponding to the product number.

In each control pattern, not just on and off signals but a two-stage control pattern, a multistage lighting pattern, and/or changes in color temperature are defined depending on the type of lighting device, and the pulse wave measuring apparatus 10 can automatically identify the lighting device 30. That is, the control patterns may include control patterns according to the models of the lighting device 30 and include at least one of a first control pattern, a second control pattern, a third control pattern, and a fourth control pattern. The first control pattern is a control pattern for adjusting the amount of light and color temperature. The second control pattern is a control pattern for adjusting the amount of light in one stage, namely between on and off. The third control pattern is a control pattern for adjusting the amount of light in two stages, namely using a first amount of visible light and a second amount of visible light, which is smaller than the first amount of visible light. The fourth control pattern is a control pattern for adjusting the amount of light without stages.

### Light Source Control Unit

The light source control unit 115 determines to increase, decrease, or maintain at least either the amount of visible light of the lighting device 30 or the amount of infrared light of the infrared light source 123 in accordance with a signal according to a degree of correlation and a result of extraction received from the correlation degree calculation unit 113 and outputs one of first to fourth control signals according to a result of the determination to the lighting device 30 and/or the infrared light source 123.

In addition, the light source control unit 115 obtains, from the control pattern obtaining unit 114, a control pattern used to adjust the light of the lighting device 30 and determines a timing of the adjustment of the amount of light of the visible LEDs 31, which are the light sources of the lighting device 30, and the amount of light in accordance with the obtained control pattern. More specifically, the light source control unit 115 outputs, to the lighting device 30, a visible light control signal for adjusting the amount of light of the lighting device 30 using the control pattern obtained by the control pattern obtaining unit 114 in accordance with the amount of infrared light of the infrared light source 123.

If the light source control unit 115 receives a “false” signal, the light source control unit 115 can determine that a correlation coefficient between first and second heartbeat intervals in a visible light waveform and an infrared waveform is smaller than the second threshold but the heartbeat intervals have been appropriately obtained in the two waveforms. At this time, the light source control unit 115 can determine that a signal of the infrared waveform is weak relative to a signal of the visible light waveform and that, through filtering, although certain feature points in the two waveforms can be obtained, positions of peaks do not correspond to each other because the sharpness of the peaks is low. In this case, therefore, the light source control unit 115 increases the amount of light of the infrared light source 123 until a second gradient from a top point to a bottom point in the infrared waveform becomes the first gradient A stored in the memory.

If the light source control unit 115 receives a “true” signal, the light source control unit 115 can determine that certain feature points match between the visible light waveform and the infrared waveform. The light source control unit 115 decreases the amount of visible light of the lighting device 30 and increases the amount of infrared light of the infrared light source 123 until the second gradient from the top point to the bottom point in the infrared waveform becomes the first gradient A stored in the memory. That is, if a degree of correlation is equal to or higher than the second threshold, the light source control unit 115 decreases the amount of visible light of the visible light source and increases the amount of infrared light of the infrared light source 123. The amount of infrared light is increased until the second gradient in the infrared waveform becomes the first gradient A stored in the memory (storage 103).

The processing units of the pulse wave calculation device 100 repeatedly obtain second visible light images, extract a second visible light waveform, obtain second infrared images, extract a second infrared waveform, and calculate a second correlation coefficient. In the repeated process for calculating the second correlation coefficient, the second gradient and the first gradient stored in the memory are compared with each other, and the light source control unit 115 keeps outputting an infrared control signal to the infrared light source 123 until the second gradient becomes the first gradient.

If the light source control unit 115 receives a “false, IR” signal, for example, the light source control unit 115 can determine that the infrared waveform calculation unit 112 has not appropriately obtained certain feature points in the infrared waveform. That is, the “false, IR” signal indicates, for example, that the infrared waveform includes much noise. The amount of light of the lighting device 30, therefore, is not adjusted, and the amount of light of the infrared light source 123 is increased.

That is, if it is determined as a result of the third and fourth determinations that the absolute error e that is the first time difference is larger than the sixth threshold (200 ms), the light source control unit 115 outputs an infrared control signal to the infrared light source 123. If it is determined as a result of the seventh and eighth determinations that the absolute error e that is the second time difference is larger than the sixth threshold (200 ms), the light source control unit 115 outputs an infrared control signal to the infrared light source 123. The light source control unit 115 increases the amount of light of the infrared light source 123 by outputting the infrared control signal to the infrared light source 123.

If the light source control unit 115 receives a “false, RGB” signal, the light source control unit 115 can determine that the visible light waveform calculation unit 111 has not appropriately obtained certain feature points in the visible light waveform. In this case, it is difficult for the light source control unit 115 to determine whether the infrared waveform calculation unit 112 has appropriately obtained certain feature points in the infrared waveform. If a standard deviation of heartbeat intervals in the first certain time period is equal to or smaller than the fourth threshold in the infrared waveform, therefore, the light source control unit 115 decreases the amount of light of the lighting device 30 and increases the amount of light of the infrared light source 123 until a gradient from a top point to a bottom point in the infrared waveform becomes the first gradient A. If the standard deviation in the infrared waveform exceeds the fourth threshold, the light source control unit 115 determines that both signals have not been obtained, and changes the signal to “false, both”.

That is, if it is determined as a result of the fifth determination that the second standard deviation is equal to or smaller than the fourth threshold, the light source control unit 115 outputs a visible light control signal to the lighting device 30 and an infrared control signal to the infrared light source 123. If the second standard deviation is larger than the fourth threshold, the light source control unit 115 outputs a third control signal to the lighting device 30 and a fourth control signal to the infrared light source 123. As described above, the fifth determination is made after it is determined as a result of the third and fourth determinations that the first time difference is smaller than the fifth threshold and used to determine whether the second standard deviation is equal to or smaller than the fourth threshold.

If it is determined as a result of the ninth determination that the fourth standard deviation is equal to or smaller than the fourth threshold, the light source control unit 115 outputs a visible light control signal to the lighting device 30 and an infrared control signal to the infrared light source 123. If it is determined as a result of the ninth determination that the fourth standard deviation is larger than the fourth threshold, the light source control unit 115 outputs a third control signal to the lighting device 30 and a fourth control signal to the infrared light source 123. As described above, the ninth determination is made after it is determined as a result of the seventh and eighth determinations that the second time difference is smaller than the fifth threshold and used to determine whether the fourth standard deviation is equal to or smaller than the fourth threshold.

If the light source control unit 115 receives a “false, both” signal, the light source control unit 115 can determine that certain feature points have not been obtained in both the visible light waveform and the infrared waveform. In this case, the light source control unit 115 increases the amount of light of the lighting device 30 until a gradient from a top point to a bottom point in the visible light waveform becomes the first gradient A. If an initial amount of light in the visible light waveform is stored in the memory, the light source control unit 115 may increase the amount of light of the lighting device 30 until the initial amount of light is achieved. In addition, the light source control unit 115 decreases the amount of light of the infrared light source 123 to zero. That is, if certain feature points have not been obtained in both the visible light waveform and the infrared waveform, the light source control unit 115 resets the amount of light of the lighting device 30 and the amount of light of the infrared light source 123 to initial states, in which certain feature points can be most certainly obtained, and restarts the adjustment of the amount of light.

That is, if it is determined as a result of the third and fourth determinations that the absolute error e that is the first time difference is equal to or larger than the fifth threshold but equal to or smaller than the sixth threshold, the light source control unit 115 outputs a third control signal to the lighting device 30 and a fourth control signal to the infrared light source 123. If it is determined as a result of the seventh and eighth determinations that the absolute error e that is the second time difference is equal to or larger than the fifth threshold but equal to or smaller than the sixth threshold, the light source control unit 115 outputs a third control signal to the lighting device 30 and a fourth control signal to the infrared light source 123. The light source control unit 115 increases the amount of light of the lighting device 30 by outputting the third control signal to the lighting device 30 and decreases the amount of light of the infrared light source 123 by outputting the fourth control signal to the infrared light source 123.

That is, if a standard deviation of a plurality of first heartbeat intervals exceeds the fourth threshold, a standard deviation of a plurality of second heartbeat intervals exceeds the fourth threshold, and a difference between one of the first heartbeat intervals and one of the second heartbeat intervals temporally corresponding to each other is smaller than the fifth threshold (−1× third threshold), the light source control unit 115 decreases the amount of visible light of the lighting device 30 and increases the amount of infrared light of the infrared light source 123. The light source control unit 115 increases the amount of infrared light until the second gradient in the infrared waveform becomes the first gradient A stored in the memory.

If the standard deviation of the plurality of first heartbeat intervals exceeds the fourth threshold, the standard deviation of the plurality of second heartbeat intervals exceeds the fourth threshold, and the difference between one of the first heartbeat intervals and one of the second heartbeat intervals temporally corresponding to each other is larger than the sixth threshold (i.e., third threshold), the light source control unit 115 increases the amount of infrared light of the infrared light source 123. The light source control unit 115 increases the amount of infrared light until the second gradient in the infrared waveform becomes the first gradient A stored in the memory.

If the standard deviation of the plurality of first heartbeat intervals exceeds the fourth threshold, the standard deviation of the plurality of second heartbeat intervals exceeds the fourth threshold, and the difference between one of the first heartbeat intervals and one of the second heartbeat intervals temporally corresponding to each other is between the fifth threshold and the sixth threshold, the light source control unit 115 increases the amount of light of the lighting device 30 and decreases the amount of infrared light of the infrared light source 123.

Although the light source control unit 115 increases the amount of light of the infrared light source 123 until the second gradient becomes the first gradient A in the above description if the light source control unit 115 receives a “false, both” signal or the like, that is, if certain feature points have not been obtained in both the visible light waveform and the infrared waveform, the operation performed by the light source control unit 115 is not limited to this. If an average luminance in ROIs exceeds a seventh threshold, namely 240, for example, the light source control unit 115 determines that the amount of light of the light source is so large that images of the user's skin are buried under noise information. An average luminance of 240 is a value on a scale of 0 to 255, and a larger value indicates a higher luminance. In this case, therefore, the light source control unit 115 can estimate that the second gradient in the infrared waveform exceeds the first gradient A, and may decrease the amount of infrared light until the second gradient becomes the first gradient A.

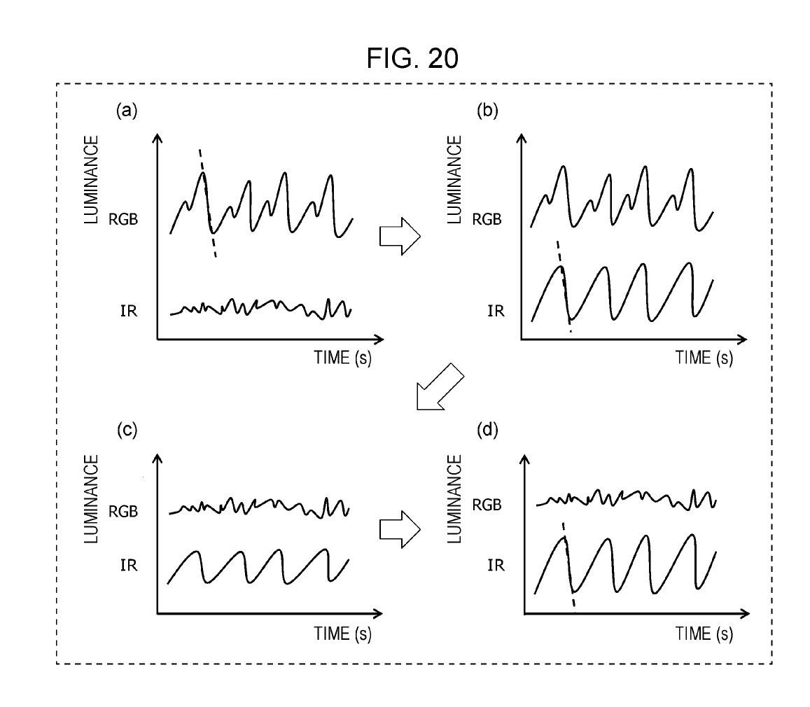

FIG. 20 is a diagram illustrating an example of simplest steps in which the pulse wave measuring apparatus 10 decreases the amount of light of the visible light source to zero and increases the amount of light of the infrared light source to an appropriate value. In all graphs of FIGS. 20(a) to 20(d), horizontal axes represent time, and vertical axes represent luminance. In FIG. 20, visible light waveforms are denoted by RGB, and infrared waveforms are denoted by IR.

FIG. 20(a) is a graph illustrating a visible light waveform and an infrared waveform obtained in an initial state, in which the user has just turned on the lighting device 30 using the pulse wave measuring apparatus 10. The visible light waveform illustrated in FIG. 20(a) has a highest gradient from a top point to a bottom point among the visible light waveforms illustrated in FIGS. 20(a) to 20(d). The gradient from the top point to the bottom point in the visible light waveform is stored in the memory as the first gradient A.

At this time, the infrared light source 123 is off. An infrared waveform, therefore, is hardly obtained. In this state, the correlation degree calculation unit 113 transmits a “false, IR” signal, for example, to the light source control unit 115. The light source control unit 115 increases the amount of light of the infrared light source 123. As the amount of light of the infrared light source 123 increases, the infrared waveform calculation unit 112 becomes able to obtain certain feature points and second heartbeat intervals from the infrared waveform. A standard deviation of the obtained second heartbeat intervals becomes equal to or smaller than the fourth threshold. As illustrated in FIG. 20(b), the amount of light of the infrared light source 123 is then increased until a second gradient from a top point to a bottom point in the infrared waveform becomes the first gradient A while keeping the standard deviation of the second heartbeat intervals equal to or smaller than the fourth threshold. After the second gradient becomes the first gradient A, the correlation degree calculation unit 113 transmits a “true: AMP=A” signal, for example, to the light source control unit 115. Upon receiving the “true: AMP=A” signal, the light source control unit 115 temporarily stops adjusting the amount of light of the infrared light source 123.

Next, in the state illustrated in FIG. 20(b), the light source control unit 115 decreases the amount of visible light of the lighting device 30. FIG. 20(c) illustrates a state in which the standard deviation of the heartbeat intervals calculated by the infrared waveform calculation unit 112 is equal to or smaller than the fourth threshold and the lighting device 30 is off. FIG. 20(d) illustrates a state in which the lighting device 30 is off and the second gradient in the infrared waveform is the first gradient A, that is, a state to be achieved.

In a process for achieving the state illustrated in FIG. 20(c) from the state illustrated in FIG. 20(b), the amount of visible light is decreased stepwise, namely, for example, 1 W at a time. Each time the amount of visible light is decreased, the infrared waveform calculation unit 112 and the correlation degree calculation unit 113 check whether certain feature points are appropriately obtained in the infrared waveform. After the infrared waveform calculation unit 112 and the correlation degree calculation unit 113 confirm that certain feature points are appropriately obtained in the infrared waveform, the amount of light of the infrared light source 123 is increased until the second gradient in the infrared waveform becomes the first gradient A as illustrated in FIG. 20(d).

In the process for achieving the state illustrated in FIG. 20(c) from the state illustrated in FIG. 20(b), the correlation degree calculation unit 113 transmits a “true” signal or a “false, IR” signal to the light source control unit 115. Each time the light source control unit 115 receives a “false, IR” signal, the light source control unit 115 adjusts the amount of light of the infrared light source 123 until a “true” signal is received. If the light source control unit 115 receives a “false, RGB” signal from the correlation degree calculation unit 113 after decreasing the amount of light of the lighting device 30, the light source control unit 115 ends the process.

In a process for achieving the state illustrated in FIG. 20(d) from the state illustrated in FIG. 20(c), the correlation degree calculation unit 113 transmits a “false, RGB” signal to the light source control unit 115. The light source control unit 115 keeps increasing the amount of light of the infrared light source 123 until the second gradient in the infrared waveform becomes the first gradient A. If the light source control unit 115 receives a “false, RGB: AMP=A” signal, which indicates that the visible light waveform has not been obtained and the second gradient has become the first gradient A, from the correlation degree calculation unit 113, for example, the light source control unit 115 ends the adjustment of the amount of light.

The light source control unit 115 adjusts the amount of light after the visible light waveform calculation unit 111 or the infrared waveform calculation unit 112 obtains two or more successive certain feature points from the visible light waveform or the infrared waveform. That is, the light source control unit 115 does not output an infrared control signal until two or more successive first peaks are extracted from a first visible light waveform in a second certain time period or two or more successive third peaks are extracted from a second visible light waveform in the second certain time period. In addition, the light source control unit 115 does not output an infrared control signal until two or more successive second peaks are extracted from a first infrared waveform in the second certain time period or two or more successive fourth peaks are extracted from a second infrared waveform in the second certain time period.

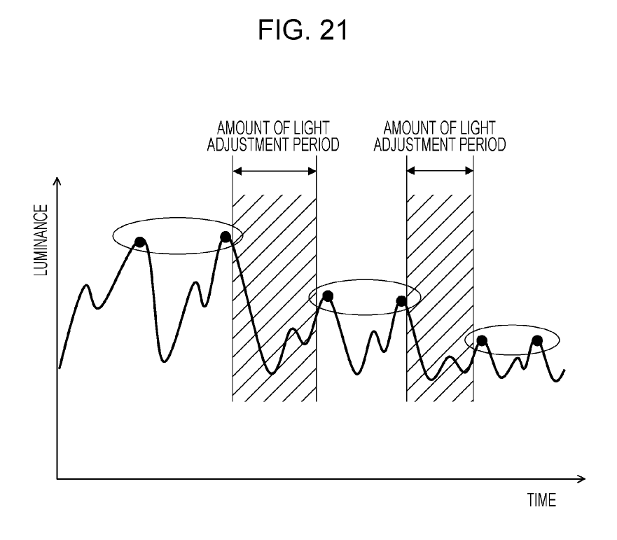

FIG. 21 is a graph illustrating the adjustment of the amount of light that is not performed until two or more successive certain feature points are extracted from a visible light waveform or an infrared waveform in the second certain time period. The graph of FIG. 21 illustrates a visible light waveform or an infrared waveform. In the graph of FIG. 21, a horizontal axis represents time, and a vertical axis represents luminance.

When the light source control unit 115 changes the amount of light of the lighting device 30 or the infrared light source 123, gain in the luminance of the visible light waveform or the infrared waveform changes. When gain in the luminance changes, positions of pulse wave timings move, thereby causing large errors in the calculation of timings of heartbeat intervals or the like. In the present disclosure, heartbeat intervals are mainly used to calculate a degree of correlation between a visible light waveform and an infrared waveform, and two successive peaks are needed to calculate the heartbeat intervals. As illustrated in FIG. 21, therefore, the light source control unit 115 adjusts the amount of light after checking that two or more successive peaks have been extracted from the visible light waveform or the infrared waveform.

The control operation of the light source control unit 115 performed when the amount of light of the lighting device 30 is adjustable without stages (i.e., the amount of light the lighting device 30 can be adjusted using the fourth control pattern) has been described. Now, a case in which the amount of light of the lighting device 30 can be adjusted using the second control pattern and a case in which the amount of light of the lighting device 30 can be adjusted using the third control pattern will be described.

A basic control operation is as described with reference to the light source control unit 115. Some characteristics cases of the operation for controlling the amount light in relation to the determinations made by the correlation degree calculation unit 113 will be described.

Unlike the case in which the fourth control pattern is used to adjust the amount of light without stages, when the amount of light of the lighting device 30 is adjusted using the second control pattern, which adjusts the amount of light in one stage, namely between on and off, or when the amount of light of the lighting device 30 is adjusted using the third control pattern, which adjusts the amount of light using the first amount of visible light and the second amount of visible light, the amount of visible light is not freely adjusted to an arbitrary value.

If the light source control unit 115 receives a “true” signal from the correlation degree calculation unit 113, for example, the light source control unit 115 increases the amount of infrared light until the second gradient in the infrared waveform becomes the first gradient A. The light source control unit 115 then outputs, to the infrared light source 123 as an infrared control signal, a control signal for increasing the amount of infrared light to a range in which certain feature points (i.e., peaks) in the infrared waveform can be detected.

After outputting the infrared control signal, the light source control unit 115 outputs, as a visible light control signal, a control signal for decreasing the amount of light of the lighting device 30 by one stage.

More specifically, when the lighting device 30 is a device whose amount of light is adjusted using the second control pattern, the light source control unit 115 outputs, as a visible light control signal, a control signal for turning off the lighting device 30.

When the lighting device 30 is a device whose amount of light is adjusted using the third control pattern and if the amount of light of the lighting device 30 is the first amount of visible light, the light source control unit 115 outputs, as a visible light control signal, a control signal for achieving the second amount of visible light, which is smaller than the first amount of visible light. When the lighting device 30 is a device whose amount of light is adjusted using the third control pattern and if the amount of light of the lighting device 30 is the second amount of visible light, the light source control unit 115 outputs, as a visible light control signal, a control signal for turning off the lighting device 30.

### First Control Pattern

Next, a case in which the amount of light and the color temperature of the lighting device 30 are adjusted will be described.

When the lighting device 30 is a device whose amount of light is adjusted using the first control pattern, in which the amount of light and the color temperature are adjusted, first, the color temperature of visible light radiated by the lighting device 30 is reduced to a certain value or lower, namely 2,500 K or lower, for example, and then the above-described operation for switching the light source is performed.

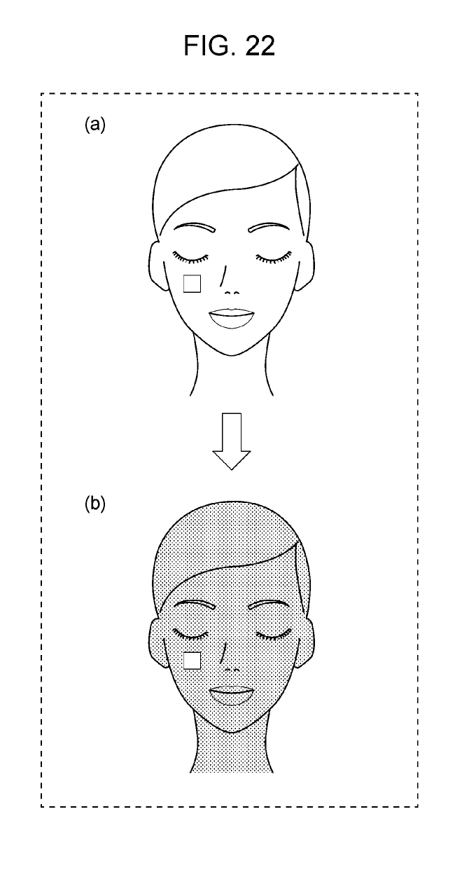

FIG. 22 is a diagram illustrating a difference in how the visible light imaging unit 122 captures an image of the user's face depending on the color temperature. FIG. 22(a) is a diagram illustrating an example of an image of the user's face under ordinary lighting, that is, for example, with the day white (about 5,000 K). FIG. 22(b) is a diagram illustrating an example of an image of the user's face with a lower color temperature, that is, for example, with the warm white (about 2,500 K). At this time, the pulse wave measuring apparatus 10 changes an algorithm used. The pulse wave measuring apparatus 10 does not obtain a visible light waveform from RGB luminance signals but uses a hue signal of a hue H calculated from RGB luminance signals.

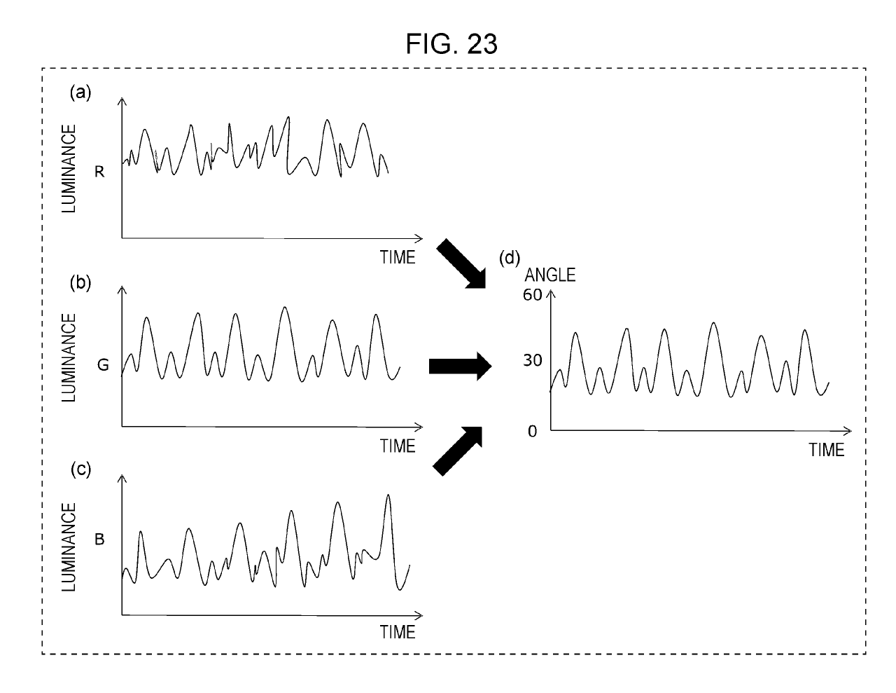

FIG. 23 is a diagram illustrating a process for calculating a hue signal of the hue H from RGB luminance signals. FIGS. 23(a) to 23(c) are graphs illustrating RGB signals (visible light waveforms) obtained by the visible light imaging unit 122. In FIGS. 23(a) to 23(c), horizontal axes represent time, and vertical axes represent R, G, or B luminance. FIG. 23(d) is a graph illustrating a signal (hue waveform) of the hue H calculated from the three signals. In FIG. 23(d), a horizontal axis represents time, and a vertical axis represents an angle in a color wheel. When the angle in the color wheel is zero, there is gain in the R signal, and there is no gain in the G and B signals. The hue signal is calculated from expression (4) on the basis of the RGB luminance signals.

$$H = 60 \times \frac{G - B}{R - B} \qquad ( 4 )$$

In expression (4), R denotes a luminance of the R signal (red signal), B denotes a luminance of the B signal (blue signal), and G denotes a luminance of the G signal (green signal).

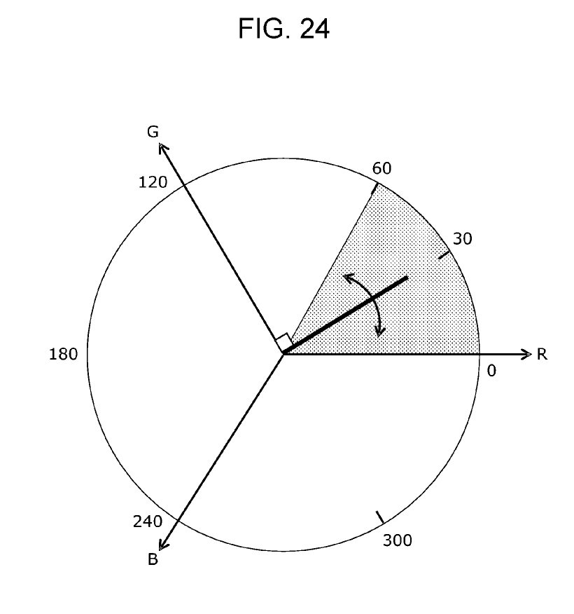

Expression (4) is an expression at a time when the luminances indicated by the luminance signals are in a relationship of R>G>B. The color of the user's skin basically satisfies this relationship, and expression (4) is applicable. When the RGB luminance signals are converted into a hue signal using expression (4), the color of the user's skin is expressed as a color located within a hue range of 0 to 60 degrees in the color wheel as illustrated in FIG. 24. That is, by using a hue signal of the hue H, not RGB luminance signals, a luminance component included in the RGB luminance signals can be eliminated, and changes in a hue component can be obtained. As a result, an effect of noise caused by changes in luminance can be reduced.

That is, the light source control unit 115 outputs, to the lighting device 30 as a color temperature control signal, a control signal for adjusting the color temperature of the lighting device 30 to a predetermined value (e.g., 2,500 K). The cloud server 1111 then calculates, using expression (4), hues obtained from third visible light images, which are obtained after the color temperature control signal is output, and extracts a visible light waveform using the calculated hues.

Furthermore, by adjusting the color temperature to 2,500 K or lower in an initial step, the user's cheeks are irradiated with reddish light like the warm white. If the visible light imaging unit 122 captures images of the user in this state, changes in the hue of a surface of the user's skin are observed at about 30 degrees in the color wheel. Because an axis of 30 degrees is perpendicular to an axis of the G signal, the hue of the surface of the user's skin is most susceptible to changes in the G signal, which sensitively indicates changes in pulse waves. By adjusting the color temperature of the lighting device 30 such that the hue of the surface of the user's skin becomes reddish, especially such that the hue H becomes close to 30 degrees, therefore, a visible light waveform can be obtained more robustly against body movement and environmental noise.

That is, after a color temperature control signal is output, the visible light waveform calculation unit 111 obtains third visible light images by capturing, in the visible light range, images of the user onto whom the lighting device 30 is radiating visible light having a predetermined color temperature. The visible light waveform calculation unit 111 then calculates hues of the obtained third visible light images and extracts a hue waveform, which indicates the user's pulse waves, from the calculated hue. The light source control unit 115 outputs, to the lighting device 30 as a color temperature control signal, a control signal for adjusting the color temperature of the lighting device 30 such that the extracted hue waveform falls within a hue range (e.g., a range of 0 to 60 degrees) extending from a certain reference value (e.g., 30 degrees in the color wheel).

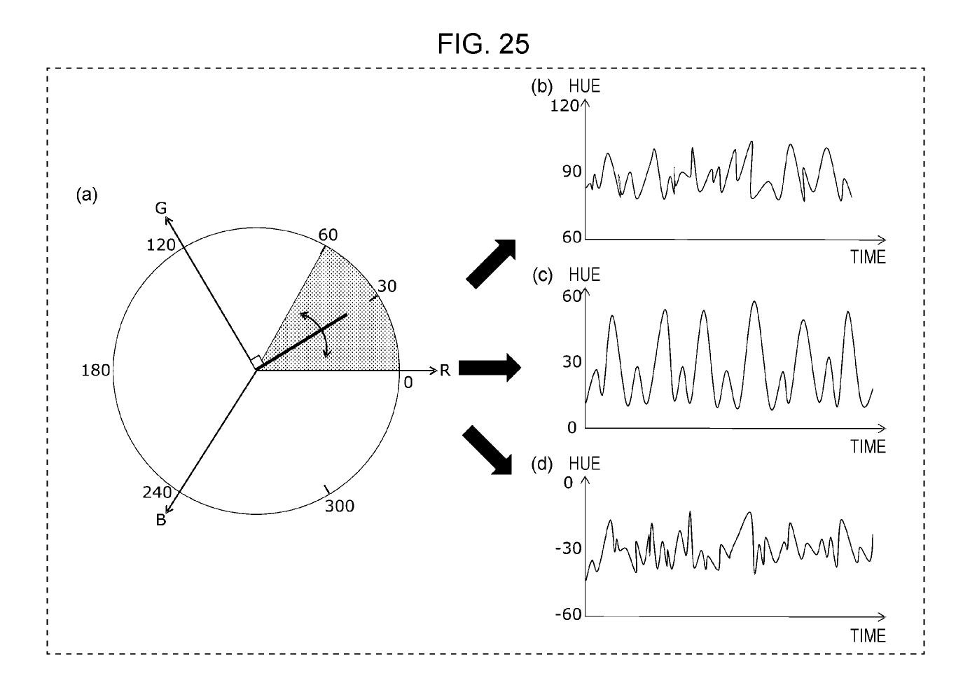

FIG. 25 is a diagram illustrating hue waveforms obtained after RGB luminance signals are converted using different hue ranges. FIG. 25(a) illustrates the color wheel. FIG. 25(b) illustrates a hue waveform obtained after the color temperature of the lighting device 30 is adjusted such that the extracted hue waveform falls within a hue range of 60 to 120 degrees in the color wheel extending from a reference value of 90 degrees. FIG. 25(c) illustrates a hue waveform obtained after the color temperature of the lighting device 30 is adjusted such that the extracted hue waveform falls within a hue range of 0 to 60 degrees in the color wheel extending from a reference value of 30 degrees. FIG. 25(d) illustrates a hue waveform obtained after the color temperature of the lighting device 30 is adjusted such that the extracted hue waveform falls within a hue range of −60 to 0 degree in the color wheel extending from a reference value of 90 degrees.

As illustrated in FIG. 25, when the color temperature of the lighting device 30 is adjusted such that the extracted hue waveform falls within the hue range of 0 to 60 degrees in the color wheel extending from the reference value of 30 degrees, a clear waveform that is hardly affected by noise caused by changes in luminance can be obtained compared to the other cases in which the color temperature is adjusted to other hue ranges.

In addition, because the warm white prompts the user to relax and fall asleep, it is advantageous for the user to change the color temperature from white (5,000 K) to red (2,500 K).

The color temperature of visible light output from the lighting device 30 may be adjusted in the following manner.

First, the control pattern obtaining unit 114 obtains the first control pattern specifying first correspondences from the lighting device 30 provided outside the pulse wave measuring apparatus 10. The first correspondences indicate a plurality of instructions and a plurality of color temperatures of visible light output from the lighting device 30. The plurality of instructions and the plurality of color temperatures are in a one-to-one relationship. The pulse wave calculation device 100 holds information indicating a first color temperature held in advance. Next, the light source control unit 115 determines a first instruction corresponding to the first color temperature while referring to the first correspondences specified by the first control pattern. Next, the light source control unit 115 outputs the first instruction to the lighting device 30. Next, the lighting device 30 radiates visible light having a color temperature corresponding to the first instruction onto the user. Next, the visible light imaging unit 122 captures, in the visible light range, a plurality of first visible light images of the user irradiated with the visible light having the color temperature corresponding to the first instruction. Next, the visible light waveform calculation unit 111 calculates a plurality of first hues from the plurality of first visible images and extracts a first hue waveform, which indicates the user's pulse waves, from the plurality of first hues. Details of the extraction of the hue waveform have been described with reference to FIG. 23 and the like. The light source control unit 115 determines whether the amplitude of the first hue waveform falls within a certain hue range. If the amplitude of the first hue waveform falls within the certain hue range, the light source control unit 115 performs the above-described operation for switching the light source. If the amplitude of the first hue wave form does not fall within the certain hue range, the light source control unit 115 performs the following process. The light source control unit 115 determines a second instruction corresponding to a second color temperature, which is different from the first color temperature, while referring to the first control pattern, and outputs the second instruction to the lighting device 30. Next, the lighting device 30 radiates visible light having a color temperature corresponding to the second instruction onto the user. Next, the visible light imaging unit 122 captures, in the visible light range, a plurality of fourth visible light images of the user irradiated with the visible light having the color temperature corresponding to the second instruction. The visible light waveform calculation unit 111 calculates a plurality of second hues from the plurality of fourth visible images and extracts a second hue waveform, which indicates the user's pulse waves, from the plurality of second hues. Details of the extraction of the hue waveform have been described with reference to FIG. 23 and the like. Next, the light source control unit 115 determines whether the amplitude of the second hue waveform falls within a certain hue range. If the amplitude of the second hue waveform fall within the certain hue range, the light source control unit 115 performs the above-described operation for switching the light source. In the operation for switching the light source, the correlation degree calculation unit 113 may extract a visible light waveform, which indicates the user's pulse waves, from the plurality of fourth visible light images, and obtain a degree of correlation on the basis of the visible light waveform and an infrared waveform, which indicates the user's pulse waves, extracted from a plurality of infrared images.

### Second Control Pattern

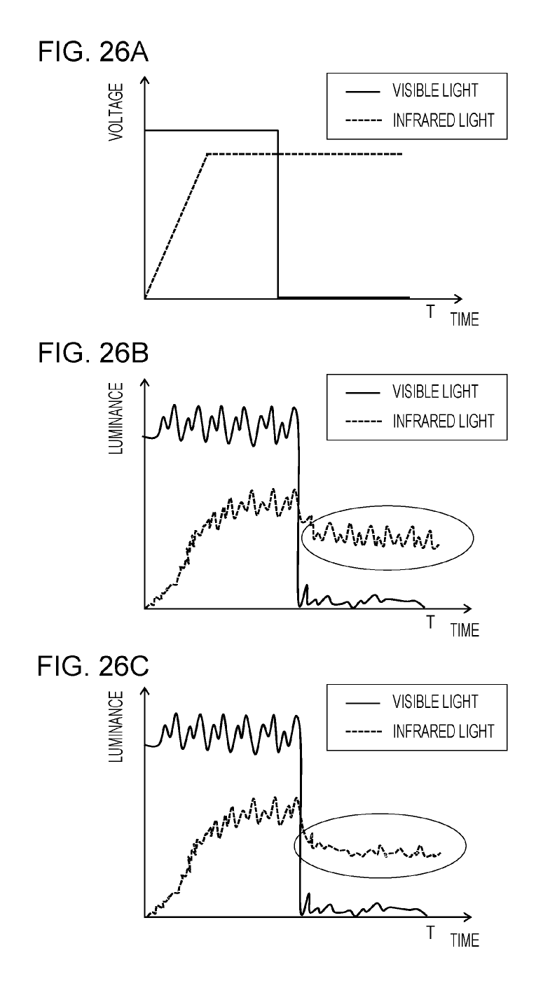

FIGS. 26A, 26B, and 26C are diagrams illustrating an operation for switching the light source in which the amount of light of a visible light source is decreased to zero and the amount of light of an infrared light source is increased to an appropriate value at a time when the lighting device 30 is a device whose amount of light is adjusted using the second control pattern. FIG. 26A is a graph illustrating changes in voltage according to the amount of light of the lighting device 30, which is the visible light source, and the amount of light of the infrared light source 123. In the graph of FIG. 26A, a horizontal axis represents time, and a vertical axis represents the voltage according to the amount of light. FIGS. 26B and 26C illustrate a visible light waveform and an infrared waveform at a time when voltages applied to the light sources are changed as illustrated in FIG. 26A. In graphs of FIGS. 26B and 26C, horizontal axes represent time, and vertical axes represent luminance.

In the operation for switching the light source from the lighting device 30, which is a light source that radiates visible light, to the infrared light source 123, time taken to complete the switching of the light source is denoted by T. The time T is, for example, 2 to 10 minutes since a beginning of the switching. In this case, a visible light waveform and an infrared waveform can be obtained more accurately and compared with each other.

When the amount of visible light of the lighting device 30 is adjusted in one stage as illustrated in FIG. 26, the amount of visible light of the lighting device 30 is either on or off. The pulse wave measuring apparatus 10 therefore needs to adjust the amount of light of the infrared light source 123 with the lighting device 30 turned on such that the user's pulse waves can be obtained under infrared light. More specifically, the light source control unit 115 outputs, to the infrared light source 123 as an infrared control signal, a control signal for increasing the amount of infrared light of the infrared light source 123 by a predetermined first value. Upon receiving the infrared control signal, the infrared light source 123 receives a certain voltage illustrated in FIG. 26B, and the amount of light of the infrared light source 123 increases by the first value as illustrated in FIGS. 26B and 26C.

The infrared camera 24, which is hardware of the infrared imaging unit 124, is affected by light in the visible light range. The pulse wave measuring apparatus 10, therefore, needs to expect a decrease in luminance received by the infrared imaging unit 124 caused when the lighting device 30 is turned off and increase the amount of light of the infrared light source 123 before the lighting device 30 is turned off.

The light source control unit 115 then outputs, to the lighting device 30 as a visible light control signal, a control signal for turning off the lighting device 30. Upon receiving the visible light control signal, the lighting device 30 turns off and no longer radiates visible light. Since the amount of light of the infrared light source 123 has been increased, the pulse wave measuring apparatus 10 can effectively obtain feature points (e.g., timings of peaks or the like) in the infrared waveform even after the lighting device 30 is turned off.

The light source control unit 115 may learn the adjustment of the amount of light of the infrared light source 123 through repeated attempts. As illustrated in FIG. 22(c), for example, feature points (e.g., peaks) in the infrared waveform might not be obtained after visible light from the lighting device 30 is turned off because a single operation for switching the light source has not increased the amount of light of the infrared light source 123 sufficiently. In this case, the light source control unit 115 increases the amount of light of the infrared light source 123 until feature points in the infrared waveform can be obtained. The light source control unit 115 may then store the amount of infrared light immediately before the lighting device 30 was turned off at a time when feature points in the infrared waveform could be obtained and the amount of light of the infrared light source 123 achieved after the lighting device 30 was turned off, and set the sum of these values of the amount of light as the amount of light of the infrared light source 123 achieved immediately before the lighting device 30 is turned off in a next operation for switching the light source. In this case, the pulse wave measuring apparatus 10 can reduce a possibility of failing to obtain an infrared waveform in each operation and can obtain the user's pulse waves during sleep more effectively.

### Third Control Pattern

Next, a case in which the amount of light of the lighting device 30 is adjusted in two stages will be described.

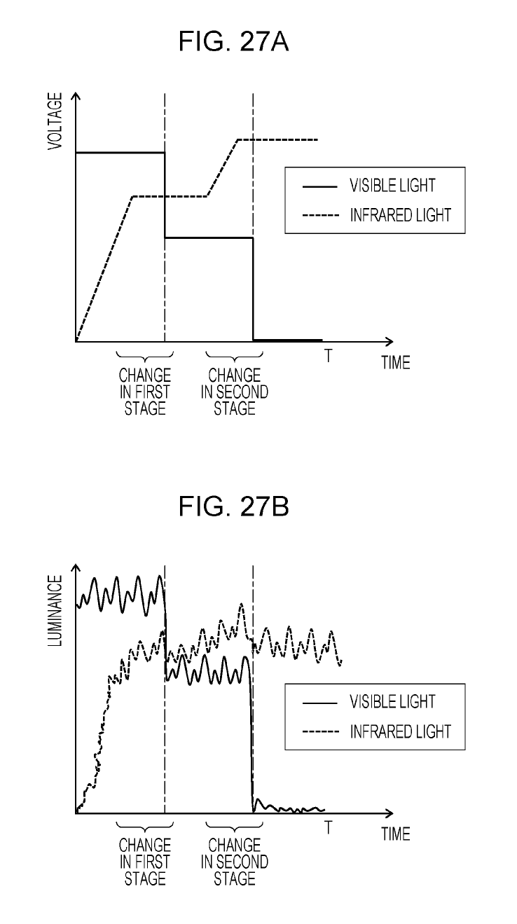

FIGS. 27 A and 27B are diagrams illustrating an operation for switching the light source at a time when the lighting device 30 is a device whose amount of light is adjusted using the third control pattern. FIG. 27A is a graph illustrating changes in voltage according to the amount of light of the lighting device 30, which is the visible light source, and the amount of light of the infrared light source 123. In the graph of FIG. 27A, a horizontal axis represents time, and a vertical axis represents the voltage according to the amount of light. FIG. 27B illustrates a visible light waveform and an infrared waveform at a time when voltages applied to the light sources are changed as illustrated in FIG. 27A. In FIG. 27B, a horizontal axis represents time, and a vertical axis represents luminance. As in FIG. 26, time taken to complete the operation for switching the light source is denoted by T.

As illustrated in FIGS. 27A and 27B, when the amount of light of the lighting device 30 is adjusted in two stages, a visible light waveform can be obtained even after the amount of visible light decreases from the first amount of visible light to the second amount of visible light, since the second amount of visible light is not zero. When the amount of light of the lighting device 30 is adjusted in two stages, first, the amount of light is adjusted. That is, the light source control unit 115 outputs, to the infrared light source 123 as an infrared control signal, a control signal for adjusting the amount of infrared light of the infrared light source 123 to a second amount of infrared light, which is larger than a first amount of infrared light by a predetermined second value. The light source control unit 115 then outputs, to the lighting device 30 as a visible light control signal, a control signal for adjusting the amount of light of the lighting device 30 from the first amount of visible light to the second amount of visible light.

Upon receiving the infrared control signal, the infrared light source 123 receives a certain voltage indicated by a change in the first stage illustrated in FIG. 27A, and the amount of light of the infrared light source 123 increases by the second value as illustrated in FIG. 27B. Upon receiving the visible light control signal, the lighting device 30 changes the amount of light thereof from the first amount of visible light to the second amount of visible light.

At this time, the pulse wave measuring apparatus 10 can identify the amount of decrease in the luminance of visible light caused by a decrease in the voltage of the lighting device 30 in the adjustment of the amount of light in the first stage. As a result, a decrease in the luminance of visible light caused by a decrease in voltage can be estimated in a next operation for adjusting the amount of light.

That is, the light source control unit 115 determines a third value by which the amount of infrared light of the infrared light source 123 is to be changed in accordance with a change in the luminance of infrared light obtained from first and second infrared images before and after an infrared control signal is output and a change in the luminance of visible light obtained from first and second visible light images before and after a visible light control signal is output.

The first infrared images are captured by the infrared imaging unit 124 before the infrared control signal is output. The second infrared images are captured by the infrared imaging unit 124 after the infrared control signal is output. The first visible light images are captured by the visible light imaging unit 122 before the visible light control signal is output. The second visible light images are captured by the visible light imaging unit 122 after the visible light control signal is output.

The third value determined here may be, for example, a value equal to or larger than a change in the luminance of infrared light that can affect infrared images when the lighting device 30 is turned off as a result of a second stage of the adjustment of the amount of light of the lighting device 30. The light source control unit 115 then outputs, to the infrared light source 123 as an infrared control signal, a control signal for adjusting the amount of light of the infrared light source 123 from the second amount of infrared light to a third amount of infrared light, which is larger than the second amount of infrared light by the determined third value. Thereafter, the light source control unit 115 outputs, to the lighting device 30 as a visible light control signal, a second-stage control signal for turning off the lighting device 30.

Upon receiving the infrared control signal, the infrared light source 123 receives a certain voltage indicated by a change in the second stage illustrated in FIG. 27A, and the amount of light of the infrared light source 123 increases by the third value as illustrated in FIG. 27B. Upon receiving the visible light control signal, the lighting device 30 turns off and no longer radiates visible light.

As described above, when the lighting device 30 is a device whose amount of light is adjusted using the third control pattern, the pulse wave measuring apparatus 10 can obtain an infrared waveform more effectively by obtaining the amount of decrease in the luminance of visible light in the first stage of the adjustment of the amount of light of the lighting device 30 and increasing the amount of light of the infrared light source 123 in accordance with the obtained amount of decrease.

As the number of stages of the adjustment of the amount of light of the lighting device 30 increases to three or more, an infrared waveform can be obtained more and more effectively by performing the above-described process in each stage.

If the light source control unit 115 receives a “false, both” signal, the light source control unit 115 resets the amount of visible light of the lighting device 30 to a highest value and performs a process for increasing the amount of light of the infrared light source 123 until the second gradient in the infrared waveform becomes the first gradient A again.

### Fourth Control Pattern

Next, a case in which the amount of light of the lighting device 30 is adjusted without stages will be described.

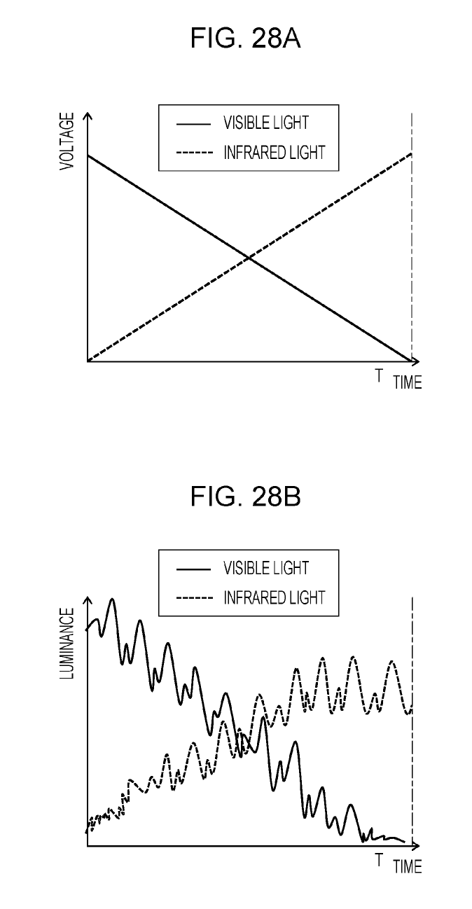

FIGS. 28A and 28B are diagrams illustrating an example of an operation for switching the light source at a time when the lighting device 30 is a device whose amount of light is adjusted using the fourth control pattern. FIG. 28A is a graph illustrating changes in voltage according to the amount of light of the lighting device 30, which is the visible light source, and the amount of light of the infrared light source 123. In the graph of FIG. 28A, a horizontal axis represents time, and a vertical axis represents the voltage according to the amount of light. FIG. 28B illustrates a visible light waveform and an infrared waveform at a time when voltages applied to the light sources are changed as illustrated in FIG. 28A. In FIG. 28B, a horizontal axis represents time, and a vertical axis represents luminance.

As illustrated in FIG. 28A, when the amount of light of the lighting device 30 is adjusted without stages and the voltage applied is linearly decreased, the amount of visible light linearly decreases and becomes zero when the time T has elapsed. It is seen, on the other hand, that the amount of infrared light of the infrared light source 123 linearly increases as the voltage applied is linearly increased. At this time, as illustrated in FIG. 28B, the visible light waveform declines as the voltage changes, and the infrared waveform rises as the voltage changes. When the lighting device 30 is a device whose amount of light is adjusted using the fourth control pattern, therefore, the amount of light can be linearly increased or decreased even if a waveform to be obtained has not been successfully switched from a visible light waveform to an infrared waveform. Pulse waves, therefore, can be obtained with infrared light by finely adjusting the amount of light. Furthermore, since the amount of light of the lighting device 30 can be finely adjusted unlike with a lighting device whose amount of light is adjusted using stages, feature points in an infrared waveform can be obtained under infrared light while identifying feature points in a visible light waveform under visible light.

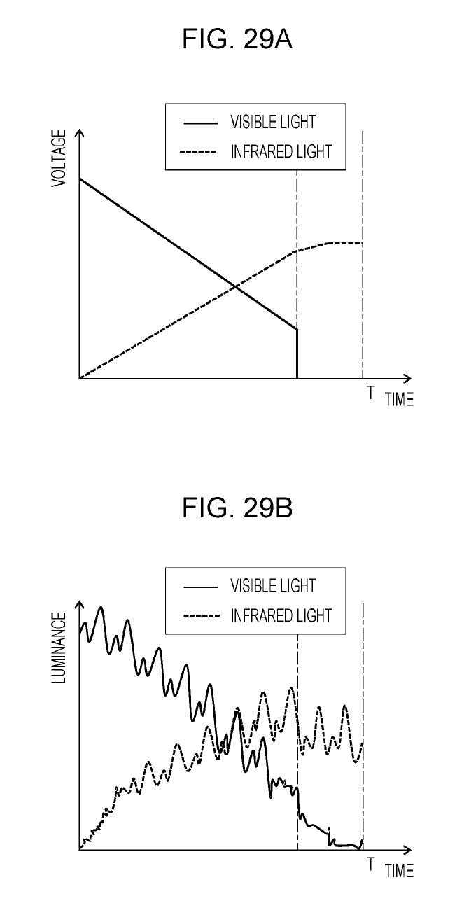

Although the amount of visible light is linearly decreased and the amount of infrared light is linearly increased when the amount of light of the lighting device 30 is adjusted without stages in the above description, how to adjust the amount of light of the lighting device 30 is not limited to this. As illustrated in FIG. 29, when illuminance achieved by the lighting device 30 falls within a certain range, namely 50 to 200 lux, and the infrared waveform calculation unit 112 has obtained feature points in the infrared waveform, for example, the light source control unit 115 may turn off the lighting device 30 whose luminance has been controlled such that the illuminance falls within the certain range. In this case, the lighting device 30 can be turned off more promptly than when the amount of light of visible light is linearly decreased to zero, thereby allowing the user to fall asleep more comfortably.

FIGS. 29A and 29B are diagrams illustrating an example of an operation for turning off the lighting device 30 when the illuminance achieved by the lighting device 30 falls within the certain range. FIG. 29A is a graph illustrating changes in voltage according to the amount of light of the lighting device 30, which is a visible light source, and the amount of light of the infrared light source 123. In FIG. 29A, a horizontal axis represents time, and a vertical axis represents the voltage according to the amount of light. FIG. 29B illustrates a visible light waveform and an infrared waveform at a time when voltages applied to the light sources are changed as illustrated in FIG. 29A. In FIG. 29B, a horizontal axis represents time, and a vertical axis represents luminance.

That is, in this case, when a degree of correlation calculated by the correlation degree calculation unit 113 is equal to or higher than the certain threshold (second threshold), the pulse wave measuring apparatus 10 outputs, to the infrared light source 123 as an infrared control signal, a control signal for increasing the amount of infrared light of the infrared light source 123 and, to the lighting device 30 as a visible light control signal, a control signal for decreasing the amount of visible light of the lighting device 30. The pulse wave measuring apparatus 10 then repeatedly obtains second visible light images, extracts a second visible light waveform, obtains second infrared images, extracts a second infrared waveform, and calculates a degree of correlation. The second visible light waveform is extracted from the second visible light images and indicates the user's pulse waves. The second infrared waveform is extracted from the second infrared images and indicates the user's pulse waves.

If the amount of light of the lighting device 30 becomes equal to or smaller than the second threshold and the degree of correlation becomes equal to or higher than the certain threshold as a result of the repeated operations for calculating the degree of correlation, the light source control unit 115 may output, to the lighting device 30 as a visible light control signal, a control signal for turning off the lighting device 30. In this case, the second threshold indicates the amount of light of the lighting device 30 at a time when the illuminance falls within the certain range.

Although the lighting device 30 sets the time taken to complete the operation for switching the light source as T in order to switch from the lighting device 30, which is a light source that radiates visible light, to the infrared light source 123 more effectively, the timing at which the switching is performed is not limited to this. In particular, when the amount of light of the lighting device 30 is adjusted without stages, the switching may be performed at an earlier timing in accordance with an instruction from the user, instead. The user might feel uncomfortable when visible light is adjusted for the time T (e.g., as long as 2 to 10 minutes) in order to switch the light source every time the user goes to sleep. As illustrated in FIG. 5B, therefore, the normal mode and the time-saving mode may be provided. If the user selects the normal mode, the pulse wave measuring apparatus 10 switches the light source in the time T. If the user selects the time-saving mode, the pulse wave measuring apparatus 10 may reduce the time taken to complete the switching operation to T/3 (e.g., 30 seconds to 3 minutes), for example, to give swiftness priority over accuracy with which a visible light waveform and an infrared waveform are obtained. The pulse wave measuring apparatus 10 may then perform the switching using the visible light waveform and the infrared waveform obtained in this period.

That is, the pulse wave measuring apparatus 10 performs either a normal process in the normal mode or a time-saving process in the time-saving mode. In the normal process, if a calculated degree of correlation is equal to or higher than the certain threshold, the pulse wave measuring apparatus 10 outputs, as an infrared control signal, a control signal for increasing the amount of infrared light of the infrared light source 123 at a first speed and, as a visible light control signal, a control signal for decreasing the amount of visible light of the lighting device 30 at a second speed. The pulse wave measuring apparatus 10 then repeatedly obtains second visible light images, extracts a second visible light waveform, obtains second infrared images, extracts a second infrared waveform, and calculates a degree of correlation. In the time-saving process, if a calculated degree of correlation is equal to or higher than the certain threshold, the pulse wave measuring apparatus 10 outputs, as an infrared control signal, a control signal for increasing the amount of infrared light of the infrared light source 123 with a third speed, which is twice or more as high as the first speed, and, as a visible light control signal, a control signal for decreasing the amount of visible light of the lighting device 30 at a fourth speed, which is twice or more as high as the second speed. The pulse wave measuring apparatus 10 then repeatedly obtains second visible light images, extracts a second visible light waveform, obtains second infrared images, extracts a second infrared waveform, and calculates a degree of correlation.

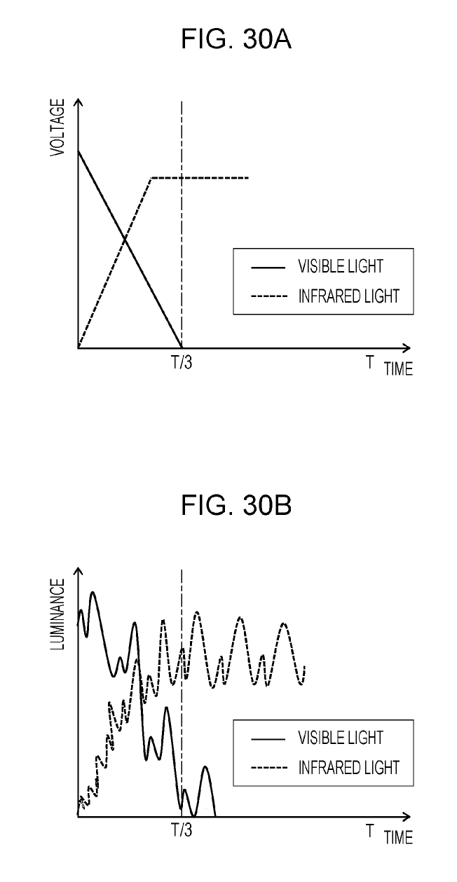

FIGS. 30A and B are diagrams illustrating an example of a case in which the switching is completed in the reduced time period. FIG. 30A is a graph illustrating changes in voltage according to the amount of light of the lighting device 30, which is a visible light source, and the amount of the infrared light source 123. In FIG. 30A, a horizontal axis represents time, and a vertical axis represents the voltage according to the amount of light. FIG. 30B illustrates a visible light waveform and an infrared waveform at a time when voltages applied to the light sources are changed as illustrated in FIG. 30A. In FIG. 30B, a horizontal axis represents time, and a vertical axis represents luminance.

As illustrated in FIG. 30A, the amount of light of the lighting device 30 becomes zero in the time T/3 after the switching starts. At this time, as illustrated in FIG. 30B, the number of peaks of the visible light waveform is smaller than the number of peaks of the visible light waveform obtained during the switching in the normal mode in which the time T is used. In the time-saving mode, therefore, the number of pieces of data regarding feature points in the visible light waveform to be compared in order to obtain an infrared waveform in the switching decreases. Although the accuracy of the switching operation decreases in this case, the time taken to complete the switching can be reduced. By performing the switching operation in the time-saving mode, the user can go to sleep promptly when he/she desires to.

### Biological Information Calculation Unit

The biological information calculation unit 116 calculates biological information regarding the user using either feature values of a visible light waveform obtained by the visible light waveform calculation unit 111 or feature values of an infrared waveform obtained by the infrared waveform calculation unit 112. More specifically, if the lighting device 30 is on and the visible light waveform calculation unit 111 can obtain a visible light waveform, the biological information calculation unit 116 obtains first heartbeat intervals from the visible light waveform calculation unit 111. The biological information calculation unit 116 then calculates biological information such as a heart rate or a stress index using the first heartbeat intervals.

If the lighting device 30 is off or the visible light waveform calculation unit 111 does not obtain a visible light waveform, and if the infrared waveform calculation unit 112 can obtain an infrared waveform, on the other hand, the biological information calculation unit 116 obtains second heartbeat intervals from the infrared waveform calculation unit 112. The biological information calculation unit 116 then similarly calculates biological information such as a heart rate or a stress index using the second heartbeat intervals.

If both the visible light waveform calculation unit 111 and the infrared waveform calculation unit 112 can extract feature values (heartbeat intervals) from waveforms (a visible light waveform and an infrared waveform), the biological information calculation unit 116 calculates biological information using the first heartbeat intervals from the visible light waveform calculation unit 111. This is because robustness against noise such as body movement and resultant reliability are higher with visible light than with infrared light.

The biological information calculation unit 116 may calculate biological information using feature values of an obtained visible light waveform or using feature values of an obtained infrared waveform. The biological information calculation unit 116 may calculate biological information regarding the user using feature values of a second visible light waveform obtained after the light source control unit 115 outputs second control information or using feature values of a first visible light waveform obtained before the light source control unit 115 outputs the second control information. Similarly, the biological information calculation unit 116 may calculate biological information regarding the user using feature values of a second infrared waveform obtained after the light source control unit 115 outputs an infrared control signal or using feature values of a first infrared waveform obtained before the light source control unit 115 outputs the infrared control signal.

Although the biological information to be calculated is a heart rate or a stress index in the above description, the biological information to be calculated is not limited to these. For example, an acceleration pulse wave may be calculated from obtained pulse waves in order to obtain an arteriosclerosis index, instead. Alternatively, timings of pulse waves may be accurately obtained from two different parts of the user's body, and blood pressure may be estimated from a difference (pulse wave velocity) between the timings. Alternatively, the dominance of a sympathetic nervous system or a parasympathetic nervous system may be calculated from variation in heartbeat intervals in order to obtain the depth of sleep.

As a stress index, the biological information calculation unit 116 may output information indicating that stress is high or low on the basis of low frequency and high frequency (LF/HF).

The biological information calculation unit 116 can obtain the depth of sleep in a manner described in Japanese Unexamined Patent Application Publication No. 2007-130182. More specifically, the depth of sleep can be determined on the basis of the LF/HF and presence or absence of body movement. The depth of sleep is an index indicating a degree of activity of a subject's brain. For example, the depth of sleep may be identified as non-rapid eye movement sleep or rapid eye movement sleep. The non-rapid eye movement sleep may be further divided into shallow sleep and deep sleep.

The biological information calculation unit 116 may give a value to each stage of the depth of sleep and output the value as the depth of sleep.

The LF and the HF can be obtained by performing a process described in Japanese Unexamined Patent Application Publication No. 2007-130182. That is, pulse interval data (heartbeat intervals) is converted into frequency spectrum distribution, for example, through a fast Fourier transform (FFT). Next, the LF and the HF are obtained from the obtained frequency spectrum distribution. More specifically, the LF and the HF are arithmetic means of a plurality of values of the sum of three points of a plurality of power spectra, namely a peak and two points that are equally distant from the peak. Examples of a frequency analysis method other than the FFT include an autoregressive (AR) model, a maximum entropy method, and a wavelet method.

### Display Device

The display device 40 displays biological information received from the biological information calculation unit 116. More specifically, the display device 40 displays biological information, such as a heart rate, a stress index, and the depth of sleep, obtained from the biological information calculation unit 116. The display device 40 may be achieved by the mobile terminal 200, for example, and display a graphic indicating biological information on the display 204 of the mobile terminal 200 or output a speech sound indicating biological information from a speaker of the mobile terminal 200, which is not illustrated.

If the pulse wave measuring apparatus 10 includes a display, the display device 40 may be achieved by the display. If the pulse wave measuring apparatus 10 includes a speaker, the display device 40 may be achieved by the speaker.

Although the display device 40 displays biological information obtained by the biological information calculation unit 116 in the above description, the information displayed by the display device 40 is not limited to this. For example, the display device 40 may display the amount of light of the lighting device 30 or the amount of light of the infrared light source 123, instead. Alternatively, the display device 40 may display a current degree of correlation obtained from the correlation degree calculation unit 113 in percentage as reliability. More specifically, the display device 40 may display a correlation coefficient between a visible light waveform and an infrared waveform.

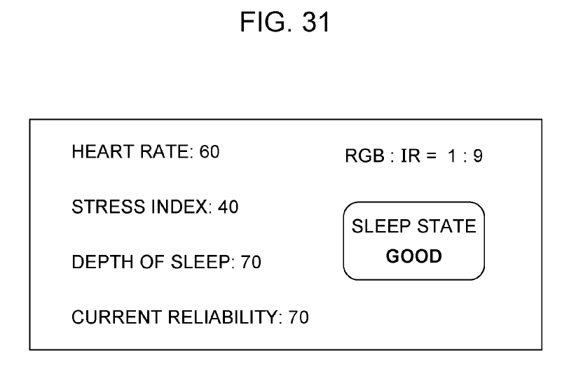

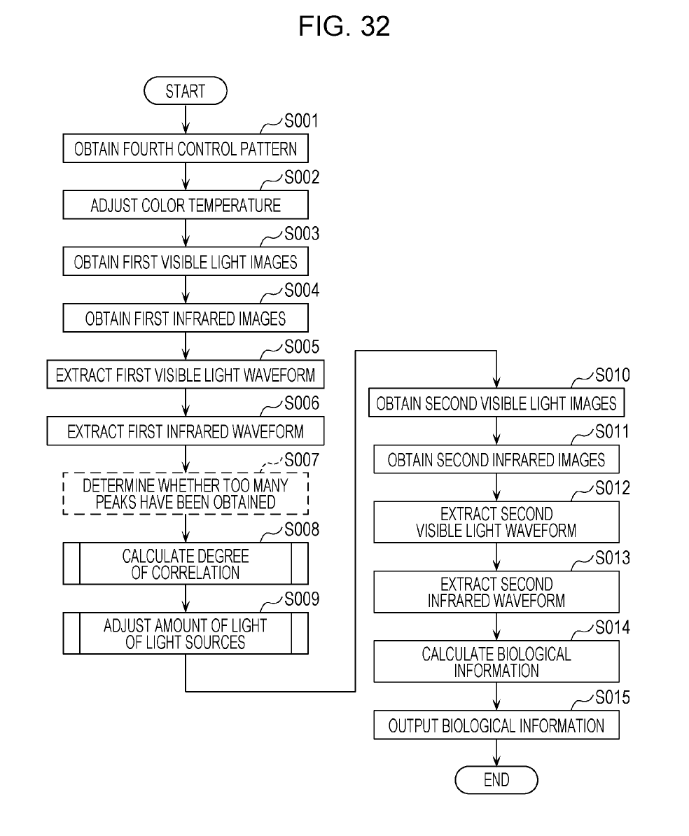

FIG. 31 is a diagram illustrating an example of a screen of the display device 40. As illustrated in FIG. 31, the display device 40 displays a graphic indicating a heart rate, a stress index, the depth of sleep, and current reliability (i.e., a correlation coefficient between heartbeat intervals in a visible light waveform and heartbeat intervals in an infrared waveform). In addition, the display device 40 may display a current ratio of the amount of visible light to the amount of infrared light. In addition, the display device 40 may determine the user's sleep state on the basis of these parameters by referring to a table in which the heart rate, the stress index, the depth of sleep, and the sleep state are associated with one another, and display the determined sleep state. If the heart rate is 65 or lower, the stress index is 40 or smaller, and the depth of sleep is 70 or larger, for example, the display device 40 displays “GOOD”. Alternatively, the display device 40 need not display information such as biological information immediately after the information is calculated. That is, because the user is usually asleep when the information is calculated, the calculated information such as biological information need not be displayed immediately after the calculation but may be recorded (accumulated) and displayed after the user wakes up in the morning. In this case, the user can check whether he/she has had a good sleep immediately after he/she wakes up.

### 1-3. Operation

Next, the operation of the pulse wave measuring apparatus 10 according to the present embodiment will be described. FIG. 32 is a flowchart illustrating a process performed by the pulse wave measuring apparatus 10 according to the present embodiment.

First, the user enters a room or performs an operation to activate the lighting device 30.

The light source control unit 115 obtains the fourth control pattern from the lighting device 30 (S001).

The light source control unit 115 outputs a visible light control signal on the basis of the obtained fourth control pattern to the lighting device 30 to adjust the color temperature of visible light of the lighting device 30 such that an extracted hue waveform falls within a hue range (e.g., a range of 0 to 60 degrees) extending from a certain reference value (e.g., 30 degrees in the color wheel) (S002).

The visible light waveform calculation unit 111 obtains second visible light images by capturing, in the visible light range, images of the user onto whom the lighting device 30 is radiating visible light (S003).

The infrared waveform calculation unit 112 obtains first infrared images obtained by capturing, in the infrared range, images of the user onto whom the infrared light source 123 is radiating infrared light (S004).

The visible light waveform calculation unit 111 extracts a first visible light waveform, which indicates the user's pulse waves, from the obtained second visible light images (S005). The visible light waveform calculation unit 111 extracts a plurality of first feature points, which are certain feature points, from the visible light waveform. The visible light waveform calculation unit 111 then calculates first heartbeat intervals as feature values of the visible light waveform. The visible light waveform calculation unit 111 stores a gradient from a top point to a bottom point in the visible light waveform in the memory as the first gradient A.

The infrared waveform calculation unit 112 extracts a first infrared waveform, which indicates the user's pulse waves, from the obtained first infrared images (S006). The infrared waveform calculation unit 112 extracts a plurality of second feature points, which are certain feature points, from the infrared waveform. The infrared waveform calculation unit 112 then calculates second heartbeat intervals as feature values of the infrared waveform.

The correlation degree calculation unit 113 determines whether too many peaks have been obtained (S007). More specifically, the correlation degree calculation unit 113 determines whether there are too many peaks with respect to the first feature points extracted from the visible light waveform. The correlation degree calculation unit 113 also determines whether there are too many peaks with respect to the second feature points extracted from the infrared waveform. Details of the process for determining whether too many peaks have been obtained performed by the correlation degree calculation unit 113 will be described later.

Next, the correlation degree calculation unit 113 calculates a degree of correlation between the visible light waveform and the infrared waveform (S008). Details of the process for calculating a degree of correlation performed by the correlation degree calculation unit 113 will be described later.

Next, the light source control unit 115 adjusts the amount of light of the light sources (S009). The light source control unit 115 outputs control signals for adjusting the amount of light of the light sources in accordance with results of the adjustment of the amount of light. Details of the process for adjusting the amount of light of the lighting device 30 and the infrared light source 123 performed by the light source control unit 115 will be described later.

Next, after the adjustment of the amount of light of the light sources is completed, steps S003 to S006 are repeated as steps S010 to S013.

Next, the biological information calculation unit 116 calculates biological information from at least either the feature points of the visible light waveform or the feature points of the infrared waveform (S014).

Next, the biological information calculation unit 116 outputs the calculated biological information to the display device 40 (S015).

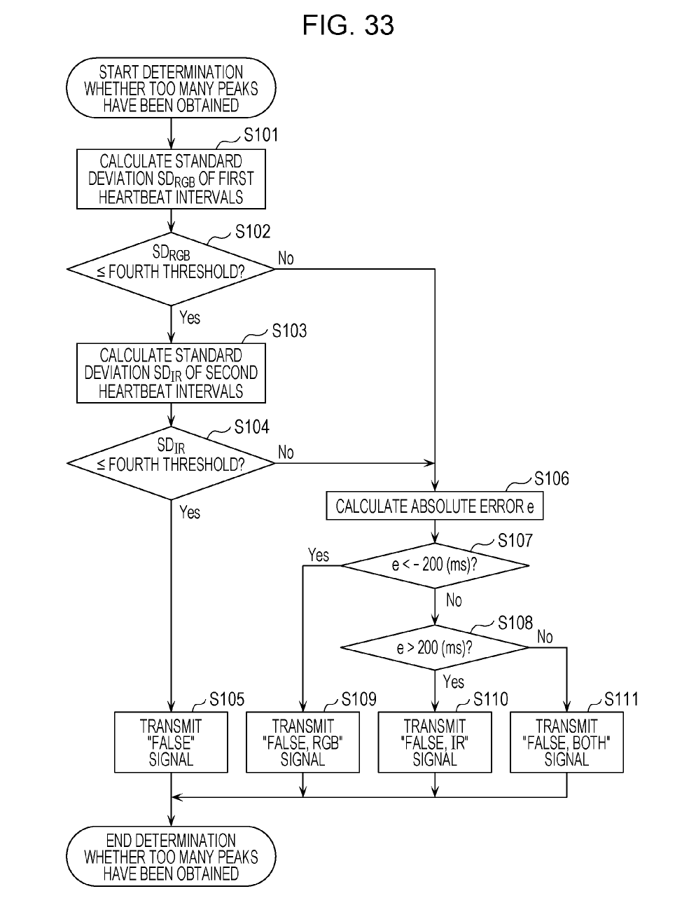

FIG. 33 is a flowchart illustrating the details of the process for determining whether too many peaks have been obtained according to the present embodiment.

The correlation degree calculation unit 113 calculates a standard deviation SD_{RGB} of first heartbeat intervals (S101).

Next, the correlation degree calculation unit 113 determines whether the standard deviation SD_{RGB} is equal to or smaller than the fourth threshold (S102).

If determining that the standard deviation SD_{RGB} is equal to or smaller than the fourth threshold (YES in S102), the correlation degree calculation unit 113 calculates a standard deviation SD_{IR} of the second heartbeat intervals (S103).

The correlation degree calculation unit 113 then determines whether the standard deviation SD_{IR} is equal to or smaller than the fourth threshold (S104).

The correlation degree calculation unit 113 thus makes the second determination for determining whether the calculated standard deviation SD_{RGB} exceeds the fourth threshold and/or whether the calculated standard deviation SD_{IR} exceeds the fourth threshold by performing at least either step S102 or S104.

If determining that the standard deviation SD_{IR} is equal to or smaller than the fourth threshold (YES in S104), the correlation degree calculation unit 113 transmits a “false” signal to the light source control unit 115 (S105).

If determining that the standard deviation SD_{RGB} exceeds the fourth threshold (NO in S102), or if determining that the standard deviation SD_{IR} exceeds the fourth threshold (NO in S104), on the other hand, the correlation degree calculation unit 113 calculates the absolute error e between one of the first heartbeat intervals and one of the second heartbeat intervals corresponding to each other (S106).

The correlation degree calculation unit 113 then determines whether the absolute error e is smaller than −200 [ms] (S107).

If determining that the absolute error e is smaller than −200 [ms] (YES in S107), the correlation degree calculation unit 113 transmits a “false, RGB” signal to the light source control unit 115 (S109).

If determining that the absolute error e is equal to or larger than −200 [ms] (NO in S107), on the other hand, the correlation degree calculation unit 113 determines whether the absolute error e is larger than 200 [ms] (S108).

That is, if determining as a result of the second determination that the standard deviation SD_{RGB} exceeds the fourth threshold and that the standard deviation SD_{IR} exceeds the fourth threshold, the correlation degree calculation unit 113 makes the third determination for determining whether the absolute error e (time difference) between one of the first heartbeat intervals and one of the second heartbeat intervals temporally corresponding to each other is smaller than the fifth threshold and the fourth determination for determining whether the time difference is larger than the sixth threshold, which is larger than the fifth threshold.

If determining that the absolute error e is larger than 200 [ms] (YES in S108), the correlation degree calculation unit 113 transmits a “false, IR” signal to the light source control unit 115 (S110).

If determining that the absolute error e is equal to or smaller than 200 [ms] (NO in S108), the correlation degree calculation unit 113 transmits a “false, both” signal to the light source control unit 115 (S111).

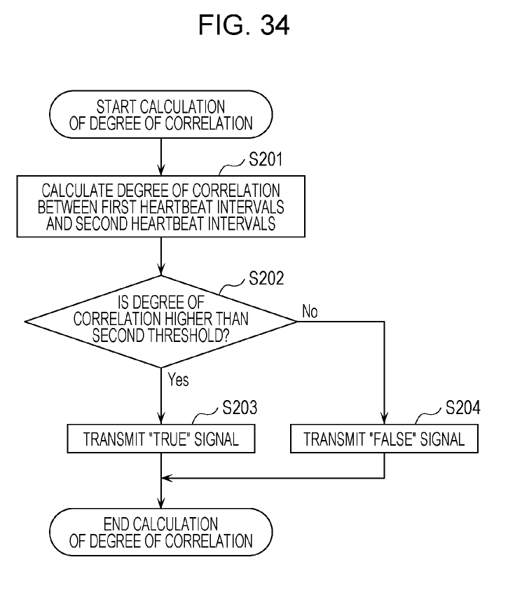

FIG. 34 is a flowchart illustrating the details of the process for calculating a degree of correlation according to the present embodiment.

First, the correlation degree calculation unit 113 calculates a degree of correlation between a plurality of first heartbeat intervals and a plurality of second heartbeat intervals (S201).

The correlation degree calculation unit 113 determines whether the calculated degree of correlation is higher than the second threshold (S202). That is, the correlation degree calculation unit 113 makes a first determination for determining whether the calculated degree of correlation is higher than the second threshold.

If determining that the degree of correlation is higher the second threshold (YES in S202), the correlation degree calculation unit 113 transmits a “true” signal to the light source control unit 115 (S203).

If determining that the degree of correlation is equal to or lower than the second threshold (NO in S202), on the other hand, the correlation degree calculation unit 113 transmits a “false” signal to the light source control unit 115 (S204).

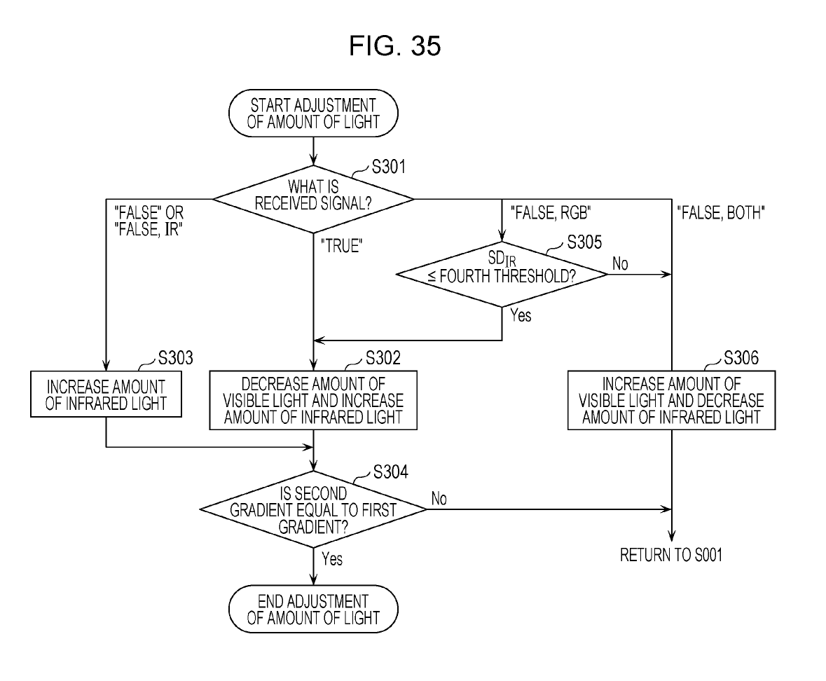

FIG. 35 is a flowchart illustrating the details of the process for adjusting the amount of light according to the present embodiment.

The light source control unit 115 determines whether a signal received from the correlation degree calculation unit 113 is a “true” signal, a “false” signal, a “false, IR” signal, a “false, RGB” signal, or a “false, both” signal (S301).

If the received signal is a “true” signal, the light source control unit 115 decreases the amount of visible light and increases the amount of infrared light (S302).

If the received signal is a “false” signal or a “false, IR” signal, the light source control unit 115 increases the amount of infrared light (S303). That is, since the light source control unit 115 receives a “false, IR” signal if the correlation degree calculation unit 113 determines that the absolute error e is larger than the sixth threshold, the light source control unit 115 outputs, to the infrared light source 123 as an infrared control signal, a control signal for increasing the amount of infrared light of the infrared light source 123.

If the light source control unit 115 has increased the amount of infrared light in step S302 or S303, the light source control unit 115 determines whether the second gradient in the infrared waveform is equal to the first gradient A stored in the memory (S304). If the light source control unit 115 has decreased the amount of visible light in step S302, the light source control unit 115 may determine whether the amount of visible light is zero.

If determining that the second gradient is equal to the first gradient A (YES in S304), the light source control unit 115 ends the process for adjusting the amount of light. If determining that the amount of visible light is zero, the light source control unit 115 may end the process for adjusting the amount of light.

If the received signal is a “false, RGB” signal, the light source control unit 115 determines whether the standard deviation SD_{IR} is equal to or smaller than the fourth threshold (S305). That is, if the correlation degree calculation unit 113 determines that the absolute error e is smaller than the fifth threshold, the light source control unit 115 makes the fifth determination for determining whether the standard deviation SD_{IR} is equal to or smaller than the fourth threshold.

If determining that the standard deviation SD_{IR} is equal to or smaller than the fourth threshold (YES in 305), the light source control unit 115 performs step S302. That is, if the correlation degree calculation unit 113 determines that the standard deviation SD_{IR} is equal to or smaller than the fourth threshold, the light source control unit 115 outputs a visible light control signal for decreasing the amount of visible light of the lighting device 30 to the lighting device 30 and an infrared control signal for increasing the amount of infrared light of the infrared light source 123 to the infrared light source 123.

If the received signal is a “false, both” signal, or if the light source control unit 115 determines that the standard deviation SD_{IR} is larger than the fourth threshold (NO in S305), the light source control unit 115 increases the amount of visible light to an initial value and decreases the amount of infrared light to turn off the infrared light source 123 (S306). That is, since the light source control unit 115 receives a “false, both” signal if the correlation degree calculation unit 113 determines that the absolute error e is equal to or larger than the fifth threshold but equal to or smaller than the sixth threshold, the light source control unit 115 outputs, to the lighting device 30 as a visible light control signal, a control signal for increasing the amount of visible light of the lighting device 30 and, to the infrared light source 123 as an infrared control signal, a control signal for decreasing the amount of infrared light of the infrared light source 123. Alternatively, if the correlation degree calculation unit 113 determines that the standard deviation SD_{IR} is larger than the fourth threshold, the light source control unit 115 outputs, to the lighting device 30 as a visible light control signal, a control signal for increasing the amount of visible light of the lighting device 30 and, to the infrared light source 123 as an infrared control signal, a control signal for decreasing the amount of infrared light of the infrared light source 123.

If determining in step S304 that the second gradient is different from the first gradient A (NO in S304), or if step S306 ends, the light source control unit 115 returns to step S001. That is, if the condition in step S304 is not satisfied even after the amount of visible light of the lighting device 30 and the amount of infrared light of the infrared light source 123 are adjusted, the pulse wave measuring apparatus 10 returns to step S001 to again obtain visible light images and infrared images, extract a visible light waveform and an infrared waveform, and calculate a degree of correlation. The pulse wave measuring apparatus 10 then outputs an infrared control signal and a visible light control signal in accordance with a result of the calculation of the degree of correlation performed again. That is, the obtaining of visible light images, the obtaining of infrared images, the extraction of a visible light waveform, the extraction of an infrared waveform, the calculation of a degree of correlation, the outputting of an infrared control signal, and the outputting of a visible light control signal are repeated until the condition in step S304 is satisfied. Visible light images repeatedly obtained in second and later processes are referred to as second visible light images, infrared images repeatedly obtained in the second and later processes are referred to as second infrared images, visible light waveforms repeatedly extracted in the second and later processes are referred to as second visible light waveforms, and infrared waveforms repeatedly extracted in the second and later processes are referred to as second infrared waveforms.

First visible light images, for example, are captured by the infrared waveform calculation unit 112 before a visible light control signal is output. Second visible light images are captured by the visible light imaging unit 122 after the visible light control signal is output. First infrared images are captured by the infrared imaging unit 124 before an infrared control signal is output. Second infrared images are captured by the infrared imaging unit 124 after the infrared control signal is output.

### 1-4. Advantageous Effects

With the pulse wave measuring apparatus 10 according to the present embodiment, the amount of light of the lighting device 30 is adjusted using a control pattern predetermined in the lighting device 30 in accordance with the adjustment of the amount of infrared light of the infrared light source 123. As a result, even if a commercial lighting device is used, the adjustment of the amount of visible light and the adjustment of the amount of infrared light can be appropriately performed, and biological information can be accurately calculated.

In addition, with the pulse wave measuring apparatus 10, second biological information is calculated from at least either feature values of a first visible light waveform and feature values of a first infrared waveform, and the calculated second biological information is output.

As a result, the second biological information can be calculated from at least either the feature values of the first visible light waveform and the feature values of the first infrared waveform obtained before the amount of visible light or infrared light is adjusted, and the calculated second biological information can be output.

In addition, with the pulse wave measuring apparatus 10, if the lighting device 30 is a device whose amount of light is adjusted using the first control pattern, in which the amount of light is adjusted in one stage, namely between on and off, a control signal for increasing the amount of infrared light of the infrared light source 123 by the first value is output to the infrared light source 123 as an infrared control signal, and a control signal for turning off the lighting device 30 is output to the lighting device 30 as a visible light control signal.

As a result, even if the lighting device 30 is a lighting device whose amount of light is adjusted in one stage, the adjustment of the amount of visible light and the adjustment of the amount of infrared light can be appropriately performed.

In addition, with the pulse wave measuring apparatus 10, if the lighting device 30 is a device whose amount of light is adjusted using the second control pattern, in which the amount of light is adjusted in two stages, namely using the first amount of visible light and the second amount of visible light, which is smaller than the first amount of visible light, a control signal for adjusting the amount of infrared light of the infrared light source 123 from the first amount of infrared light to the second amount of infrared light, which is larger than the first amount of infrared light by the predetermined second value, is output to the infrared light source 123 as an infrared control signal. A control signal for adjusting the amount of light of the lighting device 30 from the first amount of visible light to the second amount of visible light is output to the lighting device 30 as a visible light control signal. The third value for the amount of infrared light is determined in accordance with a change in the luminance of infrared light obtained from first and second infrared images and a change in the luminance of visible light obtained from first and second visible light images. A control signal for adjusting the amount of infrared light from the second amount of infrared light to the third amount of infrared light, which is larger than the second amount of infrared light by the third value, is output to the infrared light source 123 as an infrared control signal. A second-stage control signal for turning off the lighting device 30 is output to the lighting device 30 as a visible light control signal.

As a result, if the lighting device 30 is a device whose amount of light is adjusted using the second control pattern, the pulse wave measuring apparatus 10 can obtain an infrared waveform more effectively by obtaining the amount of decrease in the luminance of visible light in the first stage of the adjustment of the amount of light and increasing the amount of infrared light of the infrared light source 123.

In addition, with the pulse wave measuring apparatus 10, if the lighting device 30 is a device whose amount of light is adjusted using the third control pattern, in which the amount of light is adjusted without stages, and if a calculated degree of correlation is equal to or higher than the certain threshold, a control signal for increasing the amount of infrared light of the infrared light source 123 is output to the infrared light source 123 as an infrared control signal. A control signal for decreasing the amount of visible light of the lighting device 30 is output to the lighting device 30 as a visible light control signal. The obtaining of second visible light images, the extraction of a second visible light waveform, the obtaining of second infrared images, the extraction of a second infrared waveform, and the calculation of a degree of correlation are repeatedly performed. If the amount of light of the lighting device 30 becomes equal to or smaller than the second threshold, and if the degree of correlation calculated repeatedly becomes equal to or higher than the certain threshold, a control signal for turning off the lighting device 30 is output to the lighting device 30 as a visible light control signal.

As a result, the lighting device 30 can be turned off more promptly compared to when the amount of visible light is linearly decreased to zero, thereby allowing the user to fall asleep more comfortably.

In addition, with the pulse wave measuring apparatus 10, if the lighting device 30 is a device whose amount of light is adjusted using the fourth control pattern, in which the amount of light is adjusted without stages, and if the calculated degree of correlation is equal to or higher than a certain threshold, (i) a normal process, in which a control signal for increasing the amount of infrared light of the infrared light source 123 at a first speed is output to the infrared light source 123 as the infrared control signal, a control signal for decreasing the amount of visible light of the lighting device 30 by a second speed is output to the lighting device 30 as the visible light control signal, and the obtaining of second visible light images, the extraction of a second visible light waveform, the obtaining of second infrared images, the extraction of a second infrared waveform, and the calculation of a degree of correlation are repeatedly performed, or (ii) a time-saving process, in which a control signal for increasing the amount of infrared light of the infrared light source 123 at a third speed, which is twice or more higher than the first speed, is output to the infrared light source 123 as the infrared control signal, a control signal for decreasing the amount of visible light of the lighting device 30 at a fourth speed, which is twice or more higher than the second speed, is output to the lighting device 30 as the visible light control signal, and the obtaining of second visible light images, the extraction of a second visible light waveform, the obtaining of second infrared images, the extraction of a second infrared waveform, and the calculation of a degree of correlation are repeatedly performed, is performed.

As a result, time taken to complete the switching can be reduced.

In addition, with the pulse wave measuring apparatus 10, if the lighting device 30 is a device whose amount of light is adjusted using the first control pattern, in which the amount of light and the color temperature are adjusted, a control signal for adjusting the color temperature of the lighting device 30 to a predetermined value is output to the lighting device 30 as the visible light control signal, and a second visible light waveform is extracted using hues obtained from third visible light images obtained after the visible light control signal is output. In addition, after the visible light control signal is output, the pulse wave measuring apparatus 10 obtains third visible light images by capturing, in the visible light range, images of the user onto whom the lighting device 30 is radiating visible light having the predetermined color temperature, extracts a hue waveform, which indicates the user's pulse waves, from the hues of the obtained third visible light images, and outputs, to the lighting device 30 as the visible light control signal, a control signal for adjusting the color temperature of the lighting device 30 such that the extracted hue waveform falls within a range that extends from a certain reference value.

As a result, by adjusting the color temperature of the lighting device 30 such that the color of a surface of the user's skin changes from white to a reddish color, especially such that the hue H becomes close to 30 degrees, for example, a visible light waveform can be obtained more robustly against body movement and environmental noise.

In addition, with the pulse wave measuring apparatus 10, a degree of correlation between a visible light waveform obtained from visible light images of the user's pulse waves and an infrared waveform obtained from infrared images of the same pulse waves of the user's is calculated, and the amount of infrared light of the infrared light source 123 is adjusted in accordance with the degree of correlation. As a result, the amount of infrared light can be appropriately adjusted, and the biological information regarding the user can be obtained even in a dark state during sleep. Biological monitoring, therefore, can be performed in a noncontact manner during sleep without providing a biological sensor attached to the user.

In addition, with the pulse wave measuring apparatus 10, the correlation degree calculation unit 113 calculates the degree of correlation by comparing first heartbeat intervals calculated from the visible light waveform and second heartbeat intervals calculated from the infrared waveform. The degree of correlation between the visible light waveform and the infrared waveform, therefore, can be easily calculated.

In addition, with the pulse wave measuring apparatus 10, a second gradient in the infrared waveform after the amount of infrared light of the infrared light source 123 is adjusted is compared with the first gradient A stored in a memory, and it can be determined whether the amount of light of the infrared light source 123 has become appropriate.

In addition, with the pulse wave measuring apparatus 10, if the absolute error e exceeds the third threshold, a certain feature point that has served as a reference for the calculation of the first heartbeat intervals or the second heartbeat intervals with which it has been determined that the third threshold is exceeded in a waveform in which the number of certain feature points is larger is excluded from a calculation target of the heartbeat intervals. As a result, an excessive peak can be removed, and appropriate first heartbeat intervals and second heartbeat intervals can be obtained.

In addition, with the pulse wave measuring apparatus 10, whether to increase, decrease, or maintain the amount of light of the visible light source and the amount of light of the infrared light source 123 is determined in accordance with the calculated degree of correlation and results of the extraction of certain feature points from the visible light waveform and the infrared waveform, and control signals according to results of the determinations are output to the visible light source and the infrared light source 123. As a result, the amount of light of the visible light source and the amount of light of the infrared light source 123 can be appropriately adjusted.

In addition, with the pulse wave measuring apparatus 10, certain feature points are not extracted from a visible light waveform or an infrared waveform obtained while the amount of light of the lighting device 30 or the amount of light of the infrared light source 123 is being adjusted in accordance with a control signal. As a result, certain feature points can be appropriately extracted, and biological information can be accurately calculated.

In addition, with the pulse wave measuring apparatus 10, a control signal for adjusting the amount of visible light of the lighting device 30 or a control signal for adjusting the amount of infrared of the infrared light source 123 is not output until two or more successive certain feature points are extracted from the visible light waveform or the infrared waveform in the second certain time period. As a result, certain feature points can be appropriately extracted, and biological information can be accurately calculated.

### 1-5. Modifications

### 1-5-1. First Modification

Although the control pattern obtaining unit 114 obtains a control pattern by selecting one of the plurality of control patterns stored in the storage 103 of the pulse wave measuring apparatus 10 corresponding to the lighting device 30 in accordance with a product number of the lighting device 30 input from the user in the above embodiment, the control pattern obtaining unit 114 need not obtain a control pattern in this manner. For example, the control pattern obtaining unit 114 may read the control pattern corresponding to the lighting device 30 by communicating with the lighting device 30 using infrared light, instead. More specifically, the pulse wave measuring apparatus 10 may identify the control pattern corresponding to the lighting device 30 by transmitting control signals included in the plurality of control patterns using infrared light or the like and determining, using the control pattern obtaining unit 114, responses of the lighting device 30 to the transmitted signals in accordance with changes in the amount of light of the lighting device 30. In this case, the control pattern corresponding to the lighting device 30 can be automatically identified without receiving the product number from the user.

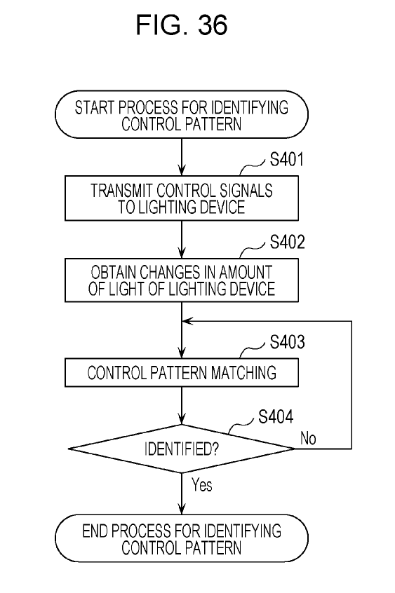

More specifically, the pulse wave measuring apparatus 10 may perform an operation illustrated in FIG. 36.

FIG. 36 is a flowchart illustrating a process for identifying a control pattern according to a modification.

The light source control unit 115 of the pulse wave measuring apparatus 10 transmits a certain control signal to the lighting device 30 (S401). The light source control unit 115 transmits one of a plurality of types of control signals to the lighting device 30. For example, the light source control unit 115 transmits one of 16-bit signals, namely “0000” to “1111”.

Next, the visible light waveform calculation unit 111 obtains changes in the amount of light of the lighting device 30 from obtained visible light images (S402).

The light source control unit 115 then performs matching in which an optimal one of the plurality of control patterns stored in advance is selected in accordance with the changes in the amount of light obtained by the visible light waveform calculation unit 111 (S403).

The light source control unit 115 continues the matching until the optimal control pattern is identified (S404).

### 1-5-2. Second Modification

Although the user can give priority to the accuracy of the obtaining of pulse waves or the swiftness of the turning off of the lighting device 30 in the switching in the above embodiment, the operation performed is not limited to this. For example, a method for controlling the light sources may be automatically changed in accordance with how many times the user has used the pulse wave measuring apparatus 10, instead.

More specifically, when the user has just made initial settings or has used the pulse wave measuring apparatus 10 about 10 times after making settings, the accuracy may be given priority. Accurate pulse waves may be obtained while carefully switching between the light sources of visible light and infrared light.

Since an environment and conditions hardly change once settings are made, on the other hand, the amount of visible light and the amount of infrared light in the operation for switching the light source may be stored in advance, and an operation in which the swiftness is given priority (that is, the switching in the time-saving mode) may be performed by finely adjusting the amount of light around the amount of visible light and the amount of infrared light stored in advance.

By carefully comparing pulse waves with each other while giving priority to the accuracy when a minimal level of accuracy is required, biological sensing can be accurately performed without interrupting the user during sleep.

As described above, since means for controlling an external lighting device can be obtained and switching between the external lighting device and an accompanying infrared light source can be performed in the present disclosure, the user can perform biological sensing during sleep in any place where there is a lighting device.

### 1-5-3. Third Modification

Although not mentioned in the above embodiment, the amount of light of the lighting device 30 may be set to a predetermined initial value when the lighting device 30 is activated. In this case, if the user prefers a certain level of illuminance or if there is a level of luminance at which the user's pulse waves can be easily obtained, the level of luminance can be immediately achieved.

### 1-5-4. Fourth Modification

Alternatively, the visible light waveform calculation unit 111 may record the amount of light of the lighting device 30 with which a visible light waveform can be obtained and a gradient from a top point to a bottom point in the visible light waveform is largest. Each time the user enters the room, the amount of light of the lighting device 30 may be adjusted to the recorded amount of light.

### 1-5-5. Fifth Modification

Although not mentioned in the above description, the user's eyesight might decreases if the user's eyes are irradiated with infrared light for a prolonged period of time. The infrared light source 123 may therefore set ROIs in parts of the user's face other than the user's eyes and radiate infrared light. When the infrared light source 123 radiates light onto the user's face, for example, pulse waves can be especially easily obtained at the user's cheeks. The light source control unit 115 may therefore identify parts under the user's eyes, for example, and cause the infrared light source 123 to radiate infrared light onto the parts. The light source control unit 115 recognizes the user's face by analyzing images captured by the infrared imaging unit 124, for example, and identifies the parts under the user's eyes using the result of the recognition. In addition, if the power of infrared light of the infrared light source 123 is equal to or higher than a certain threshold and a certain period of time has elapsed, the light source control unit 115 may adjust the amount of light of the infrared light source 123 to a value smaller than a certain value. As described above, since infrared light might affect the user's eyesight, positions of the user's cheeks may be identified through the recognition of the user's face, and radiation areas may be set such that infrared light is radiated onto the user's cheeks.

### 1-5-6. Sixth Modification

Although the pulse wave calculation device 100 is included in the pulse wave measuring apparatus 10 in the above embodiment, the configuration of the pulse wave calculation device 100 is not limited to this. For example, the pulse wave calculation device 100 may be achieved as an external server apparatus, may be achieved by the mobile terminal 200, or may be achieved by an information terminal such as a personal computer (PC), instead. That is, the pulse wave calculation device 100 may be achieved by any device insofar as images captured by the visible light imaging unit 122 and the infrared imaging unit 124 can be obtained and the amount of light of the lighting device 30 and the infrared light source 123 can be adjusted.

### 1-5-7. Seventh Modification

The components of the pulse wave measuring apparatus 10 or the like may be circuits. These circuits may together form a single circuit or may be separate circuits. These circuits may be general-purpose circuits, or may be dedicated circuits. That is, in the above embodiment, the components may be achieved by dedicated hardware or by executing software programs corresponding thereto.

Alternatively, the components may be achieved by a program execution unit, such as a CPU or a processor, that reads and executes a software program stored in a recording medium such as a hard disk or a semiconductor memory. The software program that achieves a display control method according to the above embodiment is as follows.

That is, the program causes a computer to perform a method for measuring pulse waves performed by a pulse wave measuring apparatus including a processor and a memory. The method includes obtaining, from a lighting device provided outside the pulse wave measuring apparatus, a first control pattern specifying first correspondences, which indicate color temperatures of visible light output from the lighting device corresponding to a plurality of instructions, determining a first instruction corresponding to information indicating a first color temperature held by the pulse wave measuring apparatus while referring to the first control pattern, outputting the first instruction to the lighting device, obtaining a plurality of first visible light images by capturing, in a visible light range, images of a user onto whom the lighting device is radiating visible light having the first color temperature corresponding to the first instruction, calculating a plurality of first hues from the plurality of first visible light images, extracting a first hue waveform from the plurality of first hues, determining, if amplitude of the first hue waveform does not fall within a certain hue range, a second instruction corresponding to a second color temperature, which is different from the first color temperature, while referring to the first control pattern, outputting the second instruction to the lighting device, obtaining a plurality of second visible light images, by capturing, in the visible light range, images of the user onto whom the lighting device is radiating visible light having the second color temperature corresponding to the second instruction, calculating a plurality of second hues from the plurality of second visible light images, extracting a second hue waveform from the plurality of second hues, and performing, if amplitude of the second hue waveform falls within the certain hue range, a first process, in which a plurality of first infrared images are obtained by capturing, in an infrared range, images of the user onto whom an infrared light source is radiating infrared light, a first visible light waveform, which indicates the user's pulse waves, is extracted from the plurality of second visible light images, a first infrared waveform, which indicates the user's pulse waves, is extracted from the plurality of first infrared images, a degree of correlation between the extracted first visible light waveform and the extracted first infrared waveform is calculated, an infrared control signal for adjusting an amount of infrared light of the infrared light source is output to the infrared light source in accordance with the degree of correlation, a visible light control signal for adjusting an amount of visible light of the lighting device is output to the lighting device in accordance with the degree of correlation, a plurality of third visible light images are obtained by capturing, in the visible light range, images of the user onto whom the lighting device is radiating visible light based on the visible light control signal, a plurality of second infrared images are obtained by capturing, in the infrared range, images of the user onto whom the infrared light source is radiating infrared light based on the infrared control signal, a second visible light waveform, which indicates the user's pulse waves, is extracted from the plurality of third visible light images, a second infrared waveform, which indicates the user's pulse waves, is extracted from the plurality of second infrared images, first biological information is calculated from at least either a feature value of the second visible light waveform or a feature value of the second infrared waveform, and the calculated first biological information is output.

Although the pulse wave measuring apparatus and the like according to one or a plurality of aspects have been described above on the basis of the embodiment, the present disclosure is not limited to the embodiment. The scope of the one or plurality of aspects may include modes obtained by modifying the embodiment in various ways conceivable by those skilled in the art and modes constructed by combining components in different embodiments, insofar as the scope of the present disclosure is not deviated from.

In the above embodiment, for example, a process performed by a certain component may be performed by another component. The order of steps may be changed, or a plurality of steps may be performed in parallel with each other.

The present disclosure is effective as a pulse wave measuring apparatus capable of accurately calculating biological information.

What is claimed is:

1. A pulse wave measuring apparatus comprising:
a processor,
wherein the processor obtains, from a lighting device provided outside the pulse wave measuring apparatus, a first control pattern specifying first correspondences, which indicate color temperatures of visible light output from the lighting device corresponding to a plurality of instructions,
determines a first instruction corresponding to information indicating a first color temperature held by the pulse wave measuring apparatus while referring to the first control pattern,
outputs the first instruction to the lighting device, obtains a plurality of first visible light images by capturing, in a visible light range, images of a user onto whom the lighting device is radiating visible light having the first color temperature corresponding to the first instruction,
calculates a plurality of first hues from the plurality of first visible light images,
extracts a first hue waveform from the plurality of first hues,
determines, if amplitude of the first hue waveform does not fall within a certain hue range, a second instruction corresponding to a second color temperature, which is different from the first color temperature, while referring to the first control pattern,
outputs the second instruction to the lighting device,
obtains a plurality of second visible light images, by capturing, in the visible light range, images of the user onto whom the lighting device is radiating visible light having the second color temperature corresponding to the second instruction,
calculates a plurality of second hues from the plurality of second visible light images,
extracts a second hue waveform from the plurality of second hues, and
performs, if amplitude of the second hue waveform falls within the certain hue range, a first process,
wherein the first process includes:
a plurality of first infrared images are obtained by capturing, in an infrared range, images of the user onto whom an infrared light source is radiating infrared light,
a first visible light waveform is extracted from the plurality of second visible light images,
a first infrared waveform is extracted from the plurality of first infrared images,
a degree of correlation between the extracted first visible light waveform and the extracted first infrared waveform is calculated,
an infrared control signal for adjusting an amount of infrared light of the infrared light source is output to the infrared light source in accordance with the degree of correlation,
a visible light control signal for adjusting an amount of visible light of the lighting device is output to the lighting device in accordance with the degree of correlation,
a plurality of third visible light images are obtained by capturing, in the visible light range, images of the user onto whom the lighting device is radiating visible light based on the visible light control signal,
a plurality of second infrared images are obtained by capturing, in the infrared range, images of the user onto whom the infrared light source is radiating infrared light based on the infrared control signal,
a second visible light waveform is extracted from the plurality of third visible light images,
a second infrared waveform is extracted from the plurality of second infrared images,
first biological information is calculated from at least either a feature value of the second visible light waveform or a feature value of the second infrared waveform, and
the calculated first biological information is output.

2. The pulse wave measuring apparatus according to claim 1,
wherein the certain hue range is a range of hues of 0 to 60 degrees.

3. The pulse wave measuring apparatus according to claim 2,
wherein a hue of 30 degrees serves as a reference for the certain hue range.

4. The pulse wave measuring apparatus according to claim 1,
wherein, in the calculation of the degree of correlation, the processor
(1) extracts a plurality of first peaks in a plurality of first unit periods included in a plurality of first unit waveforms, the plurality of first peaks being a plurality of first maximum points included in the plurality of first unit waveforms or a plurality of first minimum points included in the plurality of first unit waveforms, the first visible light waveform including the plurality of first unit waveforms, the plurality of first maximum points and the plurality of first unit waveforms corresponding to each other, the plurality of first minimum points and the plurality of first unit waveforms corresponding to each other, and the plurality of first unit waveforms and the plurality of first unit periods corresponding to each other,
(2) extracts a plurality of second peaks in a plurality of second unit periods included in a plurality of second unit waveforms, the plurality of second peaks being a plurality of second maximum points included in the plurality of second unit waveforms or a plurality of second minimum points included in the plurality of second unit waveforms, the first infrared waveform including the plurality of second unit waveforms, the plurality of second maximum points and the plurality of second unit waveforms corresponding to each other, the plurality of second minimum points and the plurality of second unit waveforms corresponding to each other, and the plurality of second unit waveforms and the plurality of second unit periods corresponding to each other,
(3) calculates a plurality of first heartbeat intervals on the basis of the plurality of first unit periods, the plurality of first heartbeat intervals being intervals between first time points and second time points, the plurality of first unit periods including the first time points and the second time points, and time included in the plurality of first unit periods not existing between the first time points and the second time points,
(4) calculates a plurality of second heartbeat intervals on the basis of the plurality of second unit periods, the plurality of second heartbeat intervals being intervals between third time points and fourth time points, the plurality of second unit periods including the third time points and the fourth time points, and time included in the plurality of second unit periods not existing between the third time points and the fourth time points, and
calculates the degree of correlation using a following expression (1):
$$ρ1 = \frac{{σ}_{12}}{{σ}_{1} {σ}_{2}} \qquad ( 1 )$$
ρ1: First correlation coefficient
σ₁₂: Covariance between plurality of first heartbeat intervals and plurality of second heartbeat intervals
σ₁: First standard deviation, standard deviation of plurality of first heartbeat intervals
σ₂: Second standard deviation, standard deviation of plurality of second heartbeat intervals

5. The pulse wave measuring apparatus according to claim 1,
wherein the processor calculates second biological information from at least either a feature value of the first visible light waveform and a feature value of the first infrared waveform and outputs the calculated second biological information.

6. The pulse wave measuring apparatus according to claim 1,
wherein, if the lighting device is a device whose amount of light is adjusted using a second control pattern, in which the amount of light is adjusted in one stage, namely on and off, the processor outputs, to the infrared light source as the infrared control signal, a control signal for increasing the amount of infrared light of the infrared light source by a predetermined first value and, to the lighting device as the visible light control signal, a control signal for turning off the lighting device.

7. The pulse wave measuring apparatus according to claim 1,
wherein, if the lighting device is a device whose amount of light is adjusted using a third control pattern, in which the amount of light is adjusted in two stages, namely using a first amount of visible light and a second amount of visible light, which is smaller than the first amount of visible light, the processor outputs, to the infrared light source as the infrared control signal, a control signal for adjusting the amount of infrared light of the infrared light source from a first amount of infrared light to a second amount of infrared light, which is larger than the first amount of infrared light by a predetermined second value, and, to the lighting device as the visible light control signal, a control signal for adjusting the amount of visible light of the lighting device from the first amount of visible light to the second amount of visible light, determines a third value for the amount of infrared light in accordance with a change in luminance of infrared light obtained from the first and second infrared images and a change in luminance of visible light obtained from the first and third visible light images, and outputs, to the infrared light source as the infrared control signal, a control signal for adjusting the amount of infrared light of the infrared light source from the second amount of infrared light to a third amount of infrared light, which is larger than the second amount of infrared light by the determined third value, and, to the lighting device as the visible light control signal, a second-stage control signal for turning off the lighting device.

8. The pulse wave measuring apparatus according to claim 1,
wherein, if the lighting device is a device whose amount of light is adjusted using a fourth control pattern, in which the amount of light is adjusted without stages, and if the calculated degree of correlation is equal to or higher than a certain threshold, the processor outputs, to the infrared light source as the infrared control signal, a control signal for increasing the amount of infrared light of the infrared light source and, to the lighting device as the visible light control signal, a control signal for decreasing the amount of visible light of the lighting device, repeatedly performs the obtaining of the third visible light images, the extraction of the second visible light waveform, the obtaining of the second infrared images, the extraction of the second infrared light waveform, and the calculation of a degree of correlation, and, if the amount of visible light of the lighting device becomes equal to or smaller than a second threshold, and if the degree of correlation obtained as a result of the repeatedly performed calculation of a degree of correlation becomes equal to or higher than the certain threshold, outputs, to the lighting device as the visible light control signal, a control signal for turning off the lighting device.

9. The pulse wave measuring apparatus according to claim 1,
wherein, if the lighting device is a device whose amount of light is adjusted using the fourth control pattern, in which the amount of light is adjusted without stages and if the calculated degree of correlation is equal to or higher than a certain threshold, the processor performs
(i) a normal process, in which a control signal for increasing the amount of infrared light of the infrared light source at a first speed is output to the infrared light source as the infrared control signal, a control signal for decreasing the amount of visible light of the lighting device by a second speed is output to the lighting device as the visible light control signal, and the obtaining of the third visible light images, the extraction of the second visible light waveform, the obtaining of the second infrared images, the extraction of the second infrared light waveform, and the calculation of a degree of correlation are repeatedly performed, or
(ii) a time-saving process, in which a control signal for increasing the amount of infrared light of the infrared light source at a third speed, which is twice or more higher than the first speed, is output to the infrared light source as the infrared control signal, a control signal for decreasing the amount of visible light of the lighting device at a fourth speed, which is twice or more higher than the second speed, is output to the lighting device as the visible light control signal, and the obtaining of the third visible light images, the extraction of the second visible light waveform, the obtaining of the second infrared images, the extraction of the second infrared light waveform, and the calculation of a degree of correlation are repeatedly performed.

10. A method for a pulse wave measuring apparatus, the method comprising:
obtaining, from a lighting device provided outside the pulse wave measuring apparatus, a first control pattern specifying first correspondences, which indicate color temperatures of visible light output from the lighting device corresponding to a plurality of instructions;
determining a first instruction corresponding to information indicating a first color temperature held by the pulse wave measuring apparatus while referring to the first control pattern;
outputting the first instruction to the lighting device;
obtaining a plurality of first visible light images by capturing, in a visible light range, images of a user onto whom the lighting device is radiating visible light having the first color temperature corresponding to the first instruction;
calculating a plurality of first hues from the plurality of first visible light images;
extracting a first hue waveform from the plurality of first hues;
determining, if amplitude of the first hue waveform does not fall within a certain hue range, a second instruction corresponding to a second color temperature, which is different from the first color temperature, while referring to the first control pattern;
outputting the second instruction to the lighting device;
obtaining a plurality of second visible light images, by capturing, in the visible light range, images of the user onto whom the lighting device is radiating visible light having the second color temperature corresponding to the second instruction;
calculating a plurality of second hues from the plurality of second visible light images;
extracting a second hue waveform from the plurality of second hues; and
performing, if amplitude of the second hue waveform falls within the certain hue range, a first process,
wherein the first process includes:
a plurality of first infrared images are obtained by capturing, in an infrared range, images of the user onto whom an infrared light source is radiating infrared light,
a first visible light waveform is extracted from the plurality of second visible light images,
a first infrared waveform is extracted from the plurality of first infrared images,
a degree of correlation between the extracted first visible light waveform and the extracted first infrared waveform is calculated,
an infrared control signal for adjusting an amount of infrared light of the infrared light source is output to the infrared light source in accordance with the degree of correlation,
a visible light control signal for adjusting an amount of visible light of the lighting device is output to the lighting device in accordance with the degree of correlation,
a plurality of third visible light images are obtained by capturing, in the visible light range, images of the user onto whom the lighting device is radiating visible light based on the visible light control signal,
a plurality of second infrared images are obtained by capturing, in the infrared range, images of the user onto whom the infrared light source is radiating infrared light based on the infrared control signal,
a second visible light waveform is extracted from the plurality of third visible light images,
a second infrared waveform is extracted from the plurality of second infrared images,
first biological information is calculated from at least either a feature value of the second visible light waveform or a feature value of the second infrared waveform, and
the calculated first biological information is output.

11. A recording medium storing a control program for causing a pulse wave measuring apparatus including a processor to perform a process, the recording medium being a computer-readable nonvolatile recording medium, the process comprising:
obtaining, from a lighting device provided outside the pulse wave measuring apparatus, a first control pattern specifying first correspondences, which indicate color temperatures of visible light output from the lighting device corresponding to a plurality of instructions;
determining a first instruction corresponding to information indicating a first color temperature held by the pulse wave measuring apparatus while referring to the first control pattern;
outputting the first instruction to the lighting device;
obtaining a plurality of first visible light images by capturing, in a visible light range, images of a user onto whom the lighting device is radiating visible light having the first color temperature corresponding to the first instruction;
calculating a plurality of first hues from the plurality of first visible light images;
extracting a first hue waveform from the plurality of first hues;
determining, if amplitude of the first hue waveform does not fall within a certain hue range, a second instruction corresponding to a second color temperature, which is different from the first color temperature, while referring to the first control pattern;
outputting the second instruction to the lighting device;
obtaining a plurality of second visible light images, by capturing, in the visible light range, images of the user onto whom the lighting device is radiating visible light having the second color temperature corresponding to the second instruction;
calculating a plurality of second hues from the plurality of second visible light images;
extracting a second hue waveform from the plurality of second hues; and
performing, if amplitude of the second hue waveform falls within the certain hue range, a first process,
wherein the first process includes:
a plurality of first infrared images are obtained by capturing, in an infrared range, images of the user onto whom an infrared light source is radiating infrared light,
a first visible light waveform is extracted from the plurality of second visible light images,
a first infrared waveform is extracted from the plurality of first infrared images,
a degree of correlation between the extracted first visible light waveform and the extracted first infrared waveform is calculated,
an infrared control signal for adjusting an amount of infrared light of the infrared light source is output to the infrared light source in accordance with the degree of correlation,
a visible light control signal for adjusting an amount of visible light of the lighting device is output to the lighting device in accordance with the degree of correlation,
a plurality of third visible light images are obtained by capturing, in the visible light range, images of the user onto whom the lighting device is radiating visible light based on the visible light control signal,
a plurality of second infrared images are obtained by capturing, in the infrared range, images of the user onto whom the infrared light source is radiating infrared light based on the infrared control signal,
a second visible light waveform is extracted from the plurality of third visible light images,
a second infrared waveform is extracted from the plurality of second infrared images,
first biological information is calculated from at least either a feature value of the second visible light waveform or a feature value of the second infrared waveform, and
the calculated first biological information is output.
